(19) United States
(12) Patent Application Publication
(10) Pub. No.: US 2023/0159620 A1
(43) Pub. Date: May 25, 2023

(54) COMPOSITIONS AND METHODS OF TREATMENT FOR VASCULAR DISEASES AND THROMBOSIS

(71) Applicant: Washington University, St. Louis, MN (US)

(72) Inventors: Nathan Stitziel, St. Louis, MO (US); Jared Elenbaas, St. Louis, MO (US); Arturo Alisio, St. Louis, MO (US); In-Hyuk Jung, St. Louis, MO (US)

(73) Assignee: Washington University, St. Louis, MO (US)

(21) Appl. No.: 17/978,128

(22) Filed: Oct. 31, 2022

Related U.S. Application Data
(60) Provisional application No. 63/273,739, filed on Oct. 29, 2021.

(51) Int. Cl. C07K 14/755; A61P 9/10; A61P 7/02

(52) U.S. Cl. CPC C07K 14/755; A61P 9/10; A61P 7/02; A61K 38/00

(57) ABSTRACT

Compositions and methods for treating or inhibiting a vascular disorder or thrombosis using a PEAR1 antagonist compound are disclosed.

### CROSS-REFERENCE TO RELATED APPLICATIONS

This application claims priority from U.S. Provisional Application Ser. No. 63/273,739 filed on Oct. 29, 2021, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE DISCLOSURE

The present disclosure generally relates to compositions and methods for the treatment of vascular diseases and thrombosis.

### BACKGROUND OF THE DISCLOSURE

Atherosclerosis is a chronic inflammatory disease of the arterial wall and is the leading cause of death worldwide despite growing awareness of the disease in recent years. Statin medications, through lowering plasma cholesterol, reduce myocardial infarction (MI) risk by only 30-40% suggesting there is an unmet need for identifying non-lipid therapeutic targets. Genome-wide association studies (GWAS) have identified more than 160 loci associated with the risk of coronary artery disease (CAD). At most loci, the causal gene is unknown, hindering the findings of new therapeutic targets of CAD. A large-scale association study of protein-altering variation across the genome to identify specific genes associated with CAD identified a highly conserved missense polymorphism in SVEP1 (p.D2702G) associated with increased risk of CAD in humans without any effects on lipids (Odds Ratio 1.14 per risk allele). SVEP1 p.D2702G is also associated with an increased risk of Type II diabetes, and increased systolic and diastolic blood pressure, but has no association with changes in serum lipids.

SVEP1, also known as polydom, is a large ECM protein of >300 kDa that encodes complement binding motifs, von Willebrand factor type A, EGF and EGF-like Ca²⁺ binding, and pentraxin domains, as illustrated in FIG. 12A and FIG. 12B. SVEP1 is a putative secreted protein harboring EGF domains with strong similarities to those in the Notch family, as illustrated in FIG. 13A and 13B. SVEP1 is involved in numerous developmental processes, and integrin α9β1 is the only protein known to interact with SVEP1.

SVEP1 shares structural features with selectin-family proteins, and is expressed in cultured bone marrow stromal cells and osteogenic cells. The SVEP1 promoter is known to be regulated by methylation of CpG sites. Recent studies have shown that SVEP1 is a mesenchymal factor involved in lymphatic vessel remodeling. However, the mechanisms by which SVEP1 relates to coronary atherosclerosis remain unknown.

Integrins are transmembrane receptors responsible for mediating the adhesive interactions of cells with ECM proteins and other cells. Integrins consist of an α and β subunit which are non-covalently associated with one another. For example, a subunitα9is only known to pair with β subunit β1 to form integrin Itg α9β1. Itg α9β1 is known to be expressed in airway epithelium, smooth muscle, skeletal muscle, hepatocytes, and epithelial cells. In leukocytes, Itg α9β1 is highly expressed on human neutrophils. A panel of ECM proteins, including tenascin-C and osteopontin, have been shown to serve as ligands for Itg α9β1. A recent study suggested that SVEP1 binds to Itg α9β1 with an affinity that far exceeds those of the above ligands and mediates cell adhesion in an Itg α9β1-dependent manner. However, other previous studies indicate that SVEP1 knockout mice have a more severe phenotype (death by E18.5) than Itgα9 knockout mice (death by P7), indicating that SVEP1 may signal through additional/redundant pathways unrelated to Itgα9.

Although the ECM and cell-ECM interactions are crucial for the biochemical stability of the fibrous cap overlying atherosclerotic plaque and tissue repair, the cellular and molecular mechanisms in these processes relating to plaque pathogenesis are incompletely understood.

Pearl 1 (Platelet Endothelial Aggregation Receptor 1) is an orphan receptor with no known biological ligand, despite extensive study. Pearl 1 is expressed in endothelial cells, VSMCs, and fibroblasts of coronary arteries. Pearl 1 is a tyrosine kinase-like receptor that is phosphorylated by Src family kinases (SFK). Activation of Pearl 1 results in robust activation of pAKT activation, including, but not limited to, activation of the AKT/mTOR pathway.

Other objects and features will be in part apparent and in part pointed out hereinafter.

### SUMMARY OF THE DISCLOSURE

In various aspects, the present disclosure is based on the discovery that SVEP1 is a physiological ligand of Pearl 1. As described in the examples below, SVEP1 and Pearl 1 were found to interact and activate downstream AKT/mTOR signaling.

In one aspect, a composition for treating or inhibiting a vascular disease or thrombosis within a patient in need that includes a Pearl 1 antagonist compound is disclosed. In some aspects, the Pearl 1 antagonist compound is selected from the group consisting of an antibody and a small molecule. In some aspects, the Pearl 1 antagonist compound is configured to bind to or complex with Pearl 1 produced by endothelial cells, VSMCs, fibroblasts, platelets, and any combination thereof. In some aspects, the Pearl 1 antagonist compound is further configured to block SVEP1/Pearl 1 signaling and associated downstream signaling. In some aspects, wherein the Pearl 1 antagonist compound is further configured to block the binding of SVEP1 to Pearl 1 and associated downstream signaling. In some aspects, the associated downstream signaling comprises AKT/mTOR signaling.

In another aspect, a method for treating or inhibiting a vascular disease or thrombosis within a patient in need is disclosed that includes administering a therapeutically effective amount of a Pearl 1 antagonist compound, so as to reduce availability of Pearl 1 in the subject in need. In some aspects, the Pearl 1 antagonist compound is selected from the group consisting of an antibody and a small molecule. In some aspects, wherein the Pearl 1 antagonist compound is configured to bind to or complex with Pearl 1 produced by endothelial cells, VSMCs, fibroblasts, platelets, and any combination thereof. In some aspects, wherein the Pearl 1 antagonist compound is further configured to block SVEP1/Pearl 1 signaling and associated downstream signaling. In some aspects, the Pearl 1 antagonist compound is further configured to block a binding of SVEP1 to Pearl 1 and associated downstream signaling. In some aspects, wherein the associated downstream signaling comprises AKT/mTOR signaling.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings described below are for illustrative purposes only. The drawings are not intended to limit the scope of the present teachings in any way.

FIG. 1 is a LocusZoom plot of the genetic locus containing Pearl 1 and associations with altered plasma SVEP1 (aptamer 11109.56.3) from the INTERVAL database.

FIG. 2A is a plot quantifying Pearl 1 expression in various tissues, including the tibial nerve, coronary artery, subcutaneous adipose, ecto- and endo-cervix, tibial artery, and lung.

FIG. 2B is a UMAP plot of Pearl 1 expression in various tissues, including the coronary artery.

FIG. 3A is an immunoblot that shows co-precipitation of SVEP1 and Pearl 1, consistent with a direct physical interaction between the two proteins.

FIG. 3B is another immunoblot that shows co-precipitation of SVEP1 and Pearl 1.

FIG. 3C is an immunoblot that shows endothelial cells exposed to SVEP1 have an increase in phosphorylation of Pearl 1, the first step in Pearl 1 signaling, suggesting SVEP1 signals through Pearl 1.

FIG. 4A is an immunoblot of Pearl 1 expression in endothelial cells (HUVECs), vascular smooth muscle cells (hCASMCs), and 293T cells.

FIG. 4B is a set of immunoblots of HUVECs, hCASMCs, and 293T cells, which shows that HUVECs and hCASMCs express Pearl 1 and AKT is phosphorylated when the cells are exposed to SVEP1, implying AKT phosphorylation is downstream of Pearl 1 phosphorylation. 293T cells do not express Pearl 1 and do not activate AKT signaling in response to SVEP1.

FIG. 5A is an immunoblot of human platelets, which shows SVEP1 induces AKT signaling in these cells that express high levels of Pearl 1, consistent with Pearl 1 activation when exposed to SVEP1.

FIG. 5B is an immunoblot that shows that Src is necessary for SVEP1-induced activation of AKT signaling, consistent with Pearl 1 activation and signaling.

FIG. 5C is an immunoblot that shows that endocytosis is necessary for SVEP1-induced activation of AKT signaling, consistent with Pearl 1 activation and signaling.

FIG. 5D is a set of immunoblots that shows that mTOR is activated 30 minutes after exposure to SVEP1, which has not been described before for Pearl 1.

FIG. 6A is a graph quantifying number of adhered HUVECs as a function of SVEP1 exposure concentration.

FIG. 6B is a graph quantifying the proliferation of HUVECs when treated with BSA, SVEP1, and growth media.

FIG. 6C is a set of representative images of HUVECs treated with BSA or SVEP1.

FIG. 7A is a graph quantifying platelet cells in media upon treatment with SVEP1, suggesting platelet pre-activation and clumping.

FIG. 7B is a graph quantifying receptor density (CD41, CD42b, CD61, GPVI) on isolated platelets after SVEP1 treatment.

FIG. 7C is a graph quantifying expression of PAC1 (activated integrin αIIβ3a) on isolated platelets after treatment with ADP, TRAP6, or Thrombin.

FIG. 8A is a graph quantifying the percent of CD41+CD61+ platelets in SVEP+/+ and knockout SVEPflx/flx mice, showing that the knockout platelets are less preactivated and respond less to agonists ADP and PAR4.

FIG. 8B is a graph quantifying the percent of CD62+ platelets in SVEP+/+ and knockout SVEPflx/flx mice, showing that the knockout platelets are less preactivated and respond less to agonists ADP and PAR4.

FIG. 8C is a graph quantifying receptor density (GPVI, CD61, CD42b, CD41) in platelets from SVEP1+/+ and SVEPflx/flx knockout mice.

FIG. 8D is a graph quantifying CD45+, CD41+ platelets from SVEP+/+ and SVEPflx/flx knockout mice.

FIG. 9A is a schematic of full-length SVEP1, which is necessary to activate AKT signaling.

FIG. 9B is an immunoblot for phospho-AKT upon treatment with BSA, N-terminal SVEP1, C-terminal SVEP1, and full-length SVEP1, showing the full-length SVEP1 is necessary to activate AKT signaling.

FIG. 9C is a graph quantifying whole blood platelet count upon treatment with N-terminal SVEP1, C-terminal SVEP1, and full-length SVEP1.

FIG. 10A contains representative en face views of oil red O stained plaque burden in arteries from Apoe−/− and SVEP1+/−Apoe−/− mice. The boxes indicate the aortic arch region (from the beginning of the aorta to the first intercostal artery) magnified in the left panel. FIG. 10A further includes a graph summarizing the quantification of oil red O stained area in each aortic arch and whole artery (n=15 for Apoe−/−, n=17 for SVEP1+/−Apoe−/−). Data were analyzed with unpaired nonparametric Mann-Whitney test and shown as the mean±SEM.

FIG. 10B contains representative en face views of oil red O stained aortic root cross-sections from Apoe−/− and SVEP1+/−Apoe−/− mice. FIG. 10B further includes a graph summarizing the quantification of oil red O stained areas in each aortic root cross-section (n=15 for Apoe−/−, n=17 for SVEP1+/−Apoe−/−). Scale bar, 500 μm. Data were analyzed with unpaired nonparametric Mann-Whitney test and shown as the mean±SEM.

FIG. 10C contains representative images obtained using immunofluorescent staining of aortic root sections for Mac3 from Apoe−/− and SVEP1+/−Apoe−/− mice. FIG. 10C further includes a graph summarizing quantification of Mac3 as a percentage of plaque area (n=12 for Apoe−/−, n=11 for SVEP1+/−Apoe−/−). Scale bar, 200 μm. Data were analyzed with unpaired nonparametric Mann-Whitney test and shown as the mean±SEM.

FIG. 11A contains representative en face views of oil red O stained plaque burden in arteries from SVEP1SMC +/+ and SVEP1SMC fl/fl mice groups of FIG. 3C. The boxes indicate the aortic arch region (from the beginning of the aorta to the first intercostal artery) magnified in the left panel. FIG. 11A further includes a graph summarizing the quantification of the oil red O stained area in each aortic arch and whole. Data were analyzed with unpaired nonparametric Mann-Whitney test and shown as the mean±SEM.

FIG. 11B contains representative en face views of oil red O stained aortic root cross-sections from SVEP1SMC +/+ and SVEP1SMC fl/fl mice groups. FIG. 11B further includes a graph summarizing the quantification of oil red O stained areas in each aortic root cross-section. Scale bar, 500 μm. Data were analyzed with unpaired nonparametric Mann-Whitney test and shown as the mean±SEM.

FIG. 12A contains representative images obtained using immunofluorescent staining of aortic root sections for Mac3 from SVEP1SMC +/+ and SVEP1SMC fl/fl mice. FIG. 12A further includes a graph summarizing quantification of Mac3 as a percentage of plaque area (n=14 for SVEP1SMC +/+, n=13 for SVEP1SMC fl/fl). Scale bar, 200 μm. Data were analyzed with unpaired nonparametric Mann-Whitney test and shown as the mean±SEM.

FIG. 12B contains bar graphs comparing labeling efficiency expressed as the percentage and the total number of YG-bead positive CD115+, Ly6Clow, and Ly6Chigh monocytes.

FIG. 12C contains representative images of YG-bead uptake within plaque lesions in the aortic root regions from SVEP1SMC +/+ and SVEP1SMC fl/fl mice. FIG. 12C further contains a bar graph summarizing the quantification of YG-bead uptake showing the total number of YG-beads per section (left Y-axis), and the number of YG-beads normalized to the percentage of labeled Ly6Clow monocytes (right Y-axis) (n=6 for SVEP1SMC +/+, n=7 for SVEP1SMC fl/fl). Scale bar, 50 μm.

FIG. 12D is a bar graph summarizing the results of an in vitro macrophage migration assay. Data expressed as cells per field of view. Data were analyzed with unpaired nonparametric Mann-Whitney test, and shown as the mean±SEM.

FIG. 13A contains representative images obtained using immunofluorescent staining of aortic root sections for MCM-2 from SVEP1SMC +/+ and SVEP1SMC fl/fl mice after feeding a high-fat diet for 8 weeks. Tissues were co-stained with VSMCs using anti-SMα-actin antibody. Yellow arrows indicate MCM-2+ VSMCs in plaque lesions. FIG. 13A further contains a bar graph summarizing the quantification of MCM-2+VSMCs as the total number of MCM-2 positive nuclei co-localized with SMα-actin stained VSMCs (n=15 for SVEP1SMC +/+, n=13 for SVEP1SMC fl/fl). Scale bar, 50 μm. Data were analyzed with unpaired nonparametric Mann-Whitney test and shown as the mean±SEM.

FIG. 13B is a bar graph summarizing the proliferation of SVEP1SMC fl/fl VSMCs obtained using a BrdU assay, in which all cells were incubated with pre-coated 30 μg/mL SVEP1 protein or BSA (as vehicle control) for 8 hrs., and then treated with 50 μg/mL oxLDL in the culture media for 36 hrs. Data were analyzed with a one-way ANOVA test and shown as the mean±SEM.

FIG. 13C is a bar graph summarizing the pr oliferation of Itgα9 MAC +/+ and Itgα9MAC fl/fl macrophages obtained using the BrdU assay of FIG. 13B. Incorporated BrdU was measured by ELISA assay. Data were analyzed with a one-way ANOVA test and shown as the mean±SEM.

FIG. 13D is a bar graph summarizing the phenotypic transition of SVEP1SMC +/+ and SVEP1SMC fl/fl VSMCs treated with 50 μg/mL oxLDL for 24 hrs with markers for VSMCs (Myh11 and SMα-actin) quantified. Each gene was normalized to β-actin mRNA expression. Data were analyzed with a one-way ANOVA test and shown as the mean±SEM.

FIG. 13E is a bar graph summarizing the phenotypic transition of SVEP1SMC +/+ and SVEP1SMC fl/fl VSMCs treated with 50 μg/mL oxLDL for 24 hrs with markers (Cxcl1, 11-6, and CC12) quantified. Each gene was normalized to β-actin mRNA expression. Data were analyzed with a one-way ANOVA test and shown as the mean±SEM.

FIG. 14A contains representative images showing collagen (blue colored) by Masson's trichrome staining, n=12 for Apoe−/−, n=11 for SVEP1+/−Apoe−/−. FIG. 14A also contains a bar graph summarizing a quantification of the collagen as a fraction of the plaque. Scale bar, 500 μm. Data were analyzed with unpaired nonparametric Mann-Whitney test, and shown as the mean±SEM.

FIG. 14B contains representative images showing necrotic core (indicated by yellow dotted lines) by Haemotoxylin and Eosin (H&E) staining, n=12 for Apoe−/−, n=12 for SVEP1+/−Apoe−/−. FIG. 14B also contains a bar graph summarizing a quantification of the necrotic core as a fraction of the plaque. Scale bar, 500 μm. Data were analyzed with unpaired nonparametric Mann-Whitney test, and shown as the mean±SEM.

FIG. 14C contains representative images showing infiltrated VSMCs into plaque by immunofluorescent staining with anti-SMα-actin antibody, n=12 for Apoe−/−, n=12 for SVEP1+/−Apoe−/−. FIG. 14C also contains a bar graph summarizing a quantification of the infiltrated VSMCs as a fraction of the plaque. Scale bar, 200 μm. Data were analyzed with unpaired nonparametric Mann-Whitney test, and shown as the mean±SEM.

FIG. 15 contains bar graphs summarizing a quantification of CD36, SVEP1, and Itg α9mRNA expression in VSMCs isolated from 8-week-old-SVEP1SMC +/+ and SVEP1SMC fl/fl mice after tamoxifen injection for 10 consecutive days starting at 6 weeks of age. Data were normalized to β-actin mRNA expression, and analyzed by one-way ANOVA. Data expressed as mean±SEM.

FIG. 16A is a map of the human SVEP1 protein.

FIG. 16B is a map of cleaved fragments of human SVEP1 protein

FIG. 17A is a sequence map of the human gene SVEP1.

FIG. 17B is a sequence map of the Drosophila gene uninflatable (uif).

FIG. 18 is a pair of graphs illustrating the effects of SVEP1 on VSMC proliferation.

FIG. 19 contains graphs illustrating the effect of SVEP1 depletion on various metabolic parameters in mice.

FIG. 20 is a graph summarizing the effects of SVEP1 expression in adipocytes on adipocyte volume.

FIG. 21 is a graph comparing the effects of partial and complete SVEP1 knockout on changes in body mass associated with a high-fat diet.

FIG. 22 is a graph summarizing changed in body mass associated with a high-fat diet (HFD) for Myh11Cre mice and Myh11Cre mice with myeloid cell lineage-specific knockout of Itg α9.

FIG. 23 is a graph comparing Svep1expression of primary VSMCs from Svep1SMC+/+ and Svep1SMCΔ/Δ mice with or without the addition of oxLDL for 48 hr. Increased expression of CD36, the oxLDL receptor, confirms VSMC stimulation. ***P<0.001; ****P<0.0001. Mann-Whitney test was used.

FIG. 24 is an image of Mac3 staining of aortic roots and a graph summarizing the quantification of Mac3 as a percentage of plaque area.

FIG. 25 is an image of the necrotic core outlined on H&E-stained sections and a graph summarizing the quantification of the necrotic core as a percentage of plaque area.

FIG. 26 are images of collagen staining using Masson's trichrome stain and a graph summarizing the quantification of collagen as a percentage of plaque area. All values were calculated as a percentage of plaque area. Scale bars, 200 μm. n=8-9/group. M, media; L, lumen; P, plaque. Results are shown as the mean±SEM. *P<0.05; ** P<0.01; *** P<0.001; NS, not significant.

FIG. 27A is a table summarizing the effect of the CAD-associated SVEP1 D2702G allele on plasma SVEP1 levels. The effect refers to the change per alternative allele (ending 2702G) in units of normalized protein levels after adjusting for covariates as described herein.

FIG. 27B is a genome-wide Manhattan plot for variants associated with plasma SVEP1. The —log10(p) of the association with SVEP1 levels is plotted for each variant across the genome according to chromosomal position (X-axis). The line indicates genome-wide significance (P<5×10-8). The association peak on chromosome 9 overlies the SVEP1 locus.

FIG. 27C is a graph of the estimated effect (with 95% confidence intervals) of each variant included in the Mendelian Randomization analysis on plasma SVEP1 level and CAD risk. The line demarcates the causal effect estimate (P=7×10-11).

FIG. 28 Expression of Itgα9 in the aortic root from 8-week-old Svep1SMC+/+ and Svep1SMCΔ/Δ mice using ISH. Outlined areas indicate the regions magnified in the next panels. Scale bar, 50 μm.

FIG. 29 is an image of immunoblots of integrin signaling kinases and downstream kinases of cells adhered to control, VCAM-1, or SVEP1-treated plates. β-actin was used as a loading control.

FIG. 30 is a graph summarizing the proliferation of VSMCs transfected with control or Itgα9-targetted siRNAs and grown on immobilized SVEP1 or BSA. Proliferation was determined by BrdU incorporation. *P<0.05; **P<0.01; ***P<0.001. Two-tailed t-test.

FIG. 31 is a graph summarizing the transcription of canonical Notch target genes after 4 hours of adhesion to SVEP1, relative to BSA. Two-tailed t-test.

FIG. 32 is a graph summarizing basal transcription of Notch target genes in Svep1SMC+/+ and Svep1SMCΔ/Δ VSMCs. Two-tailed t-test.

FIG. 33 is a graph summarizing the proliferation of VSMCs in response to immobilized SVEP1. Cells were treated with DMSO (carrier) or 25 μM DAPT. Proliferation was determined by BrdU incorporation. Two-tailed t-test.

FIG. 34 is a bar graph of Fgfr transcript counts from RNAseq. Each transcript is normalized to the BSA control group. Two-tailed t-test.

FIG. 35 is a graph summarizing qPCR of VSMC markers of VSMC cultured with or without 50 μg ml-l oxLDL for 24 hr. *P<0.05; **P<0.01; ***P<0.001.

FIG. 36 is a graph summarizing qPCR of inflammatory markers of VSMC cultured with or without 50 μg ml-l oxLDL for 24 hr. *P<0.05; **P<0.01; ***P<0.001.

FIG. 37A is a graph showing SVEP1 expression in atherosclerotic carotid arterial tissue, relative to adjacent, paired arterial tissue. The vertical line represents the reference tissue. Data collected from GEO GSE43292. Paired t-test.

FIG. 37B is a graph showing SVEP1 expression in medial and intimal explants from control and diabetic patients. Data collected from GEO GSE13760. Two-tailed t-test.

FIG. 37C is a graph showing the quantification of Svep1fluorescence intensity in atherosclerotic plaque.

FIG. 37D is a graph showing SVEP1 expression of CASMCs with or without the addition of oxLDL for 72 hr. Two-tailed t-test. Mann-Whitney test for (E and F). *P<0.05; ***P<0.001.

FIG. 38A contains an image showing collagen staining of an aortic root by Masson's trichrome stain, and a graph comparing collagen of Svep1SMC+/+ and Svep1SMCΔ/Δ aortas as a percentage of total plaque. n=12/group. Scale bars=200 μm. L, lumen. Data were analyzed with unpaired nonparametric Mann-Whitney test, and shown as the mean±SEM. *P<0.05; **P<0.01; NS, not significant.

FIG. 38B contains an image showing a necrotic core outlined on H&E-stained tissues, and a graph comparing necrotic cores of Svep1SMC+/+ and Svep1SMCΔ/Δ aortas as a percentage of total plaque. n=12/group. Scale bars=200 μm. L, lumen. Scale bars=200 μm. L, lumen. Data were analyzed with unpaired nonparametric Mann-Whitney test, and shown as the mean±SEM. *P<0.05; **P<0.01; NS, not significant.

FIG. 38C contains images of en face Oil Red O-stained aortas and a graph comparing quantifications of plaque based on Oil Red O-stained areas in each aortic arch and whole artery of Svep1SMC+/+ and Svep1SMCΔ/Δ mice.

FIG. 38D contains images of Oil Red O-stained aortic root cross-sections and a graph comparing quantifications of plaque based on Oil Red O-stained areas in each aortic arch and whole artery of Svep1SMC+/+ and Svep1SMCΔ/Δ mice.

FIG. 39A is a graph showing the estimated effect (with 95% confidence intervals) of each variant included in the Mendelian Randomization analysis on plasma SVEP1 level and risk of hypertension (HTN) and a histogram summarizing the estimated causal effect (with 95% confidence intervals) of each SNP included in the Mendelian Randomization analysis for a one unit increase in SVEP1 level, plotted along with the overall summary estimate from the causal analysis. The line on the left graph denotes line the causal effect estimate (P=2×10-15).

FIG. 39B is a graph showing the estimated effect (with 95% confidence intervals) of each variant included in the Mendelian Randomization analysis on plasma SVEP1 level and risk of type 2 diabetes (T2D) and a histogram summarizing the estimated causal effect (with 95% confidence intervals) of each SNP included in the Mendelian Randomization analysis for a one unit increase in SVEP1 level, plotted along with the overall summary estimate from the causal analysis. The line on the left graph denotes line the causal effect estimate (P=0.0004 for T2D).

FIG. 39C contains images of en face Oil Red O-stained aortas and a graph comparing quantifications of plaque based on Oil Red O-stained areas in each aortic arch and whole artery of ApoE−/− and Svep1G/GApoE−/− mice after 16 weeks of HFD.

FIG. 40A is a graph showing the proliferation of human CASMCs in response to immobilized SVEP1. Two-tailed t-test.

FIG. 40B is an image showing immunoblots of proximal integrin signaling kinases and downstream p38 from VSMCs adhered to control, or SVEP1, or SVEP1CADrv-treated plates. β-actin was used as a loading control. ****P<0.0001.

FIG. 41A is a graph showing qPCR determination of Cc12, Spp1, and Cxcl5 expression in aortic arches. Each gene was normalized to gapdh. Two-tailed t-test, and shown as the mean±SEM.

FIG. 41B is a graph comparing cytokine and chemokine biomarkers from 8 weeks HFD-fed Svep1SMC+/+ and Svep1SMCΔ/Δ mice. sICAM-1, soluble intercellular adhesion molecule-1; sP-selectin, soluble P-selectin; Ang-2, angiotensin-2; IL-6, interleukin-6; KC, C-X-C motif ligand1 (CXCL1); *P<0.05; **P<0.01; NS, not significant.

FIG. 42 shows maps of THP-1 cells seeded on wells containing the indicated proteins. Cells were lysed and protein lysates were subjected to immunoblotting with the indicated antibody. BSA served as a negative control and VCAM-1 served as a positive control. β-tubulin was used as a loading control.

FIG. 43A is a schematic of the SVEP1 protein. Domains were identified using the Simple Modular Architecture Research Tool (SMART). Teal, von Willebrand factor type A domain; purple, putative ephrin-receptor like; yellow, complement control protein/SUSHI repeat; orange, epidermal growth factor (EGF)-like domain or calcium-binding EGF-like domain or laminin-type EGF-like domain; scissors, putative cleavage site; P, representative phosphorylation of the Pearl 1 intracellular domain. Protein coding variants of interest are denoted at the corresponding peptide.

FIG. 43B is a schematic of the Pearl 1 protein.

FIG. 43C is a plasma SVEP1 (aptamer 11109.56.3) box and whisker plot as a function of allelic copies of rs147639000 (Pearl 1 p.D343N) in the INTERVAL database. Beta=0.67, P=6.5×10-16.

FIG. 43D is a plasma Pearl 1 (aptamer 8275.31.3) box and whisker plot as a function of allelic copies of rs147639000 (Pearl 1 p.D343N) in the INTERVAL database. Beta=−0.18, P=0.03.

FIG. 43E is a Manhattan plot of associations between Pearl 1 D343N and plasma proteins measured in INTERVAL. Each point represents the genomic location of the gene coding for a measured protein.

FIG. 43F is a plot of two-sample MR of estimated SNP effects (with 95% confidence intervals) on Pearl 1 in deCODE (x-axis) and either Pearl 1, green, or SVEP1, blue, in INTERVAL (y-axis). The causal estimate is designated by a line of the corresponding color. Pearl 1 Beta=0.86, P=2.4×10-37; SVEP1 Beta=-0.66, P=3.5×10-20.

FIG. 43G is a plot of two-sample MR of estimated SNP effects (with 95% confidence intervals) on SVEP1 in deCODE (x-axis) and either Pearl 1, green, or SVEP1, blue, in INTERVAL (y-axis). The causal estimate is designated by a line of the corresponding color. Pearl 1 Beta=-0.07, P=0.002; SVEP1 Beta=0.84, P=2.5×10-54.

FIG. 44A is a plot of biolayer interferometry sensograms. A dilution series of SVEP1 was analyzed by sensors loaded with Pearl 1ECD. The dashed line represents the end of the association step and the beginning of the dissociation step.

FIG. 44B is a set of immunoblots of the indicated proteins after co-immunoprecipitation. Negative controls included no SVEP1.

FIG. 44C is a set of immunoblots of the indicated proteins after co-immunoprecipitation. Negative controls included non-specific IgG. Additional details listed in Methods.

FIG. 44D is a plot of the expression of Pearl 1 and SVEP1 in transcripts per million (TPM) in tissues from the GTEx database. Purple circles designate adipose tissues. Orange circles designate arterial tissues. Pearson r correlation=0.74, P<0.0001.

FIG. 44E is an immunoblot analysis of Pearl 1 levels using cell lysates from the indicated cell-type. B-Tubulin served as a loading control.

FIG. 44F is a schematic of proximity or affinity-based proteomics experiments.

FIG. 44G is a list of proteins enriched in experiments represented in FIG. 44E. Hits were identified as those proteins enriched at a confidence level of P<0.10 in each experiment. Fisher's combined p-value is a meta-analysis of the four experiments.

FIG. 45A is a set of immunoblots. Isolated human platelets were exposed to immobilized BSA, SVEP1, or soluble Pearl 1pAb for 15 minutes prior to lysis. Lysates were subjected to immunoprecipitation with an anti-Pearl 1 antibody and analyzed by immunoblot assays for Pearl 1 and pTyrosine. The pTyrosine signal directly overlapped with the Pearl 1 signal at approximately 140 kDa.

FIG. 45B is a set of immunoblots. Platelets were exposed to stimuli as described in FIG. 45A. Lysates were analyzed by immunoblot assays for the indicated proteins.

FIG. 45C is a set of immunoblots. HUVECs were exposed to stimuli before lysis and analysis by immunoblot assays for the indicated proteins.

FIG. 45D is a set of immunoblots. hCASMCs were exposed to stimuli before lysis and analysis by immunoblot assays for the indicated proteins.

FIG. 45E is a set of immunoblots. 293T cells were exposed to stimuli before lysis and analysis by immunoblot assays for the indicated proteins.

FIG. 45F is a set of immunoblots. hCASMCs were transfected with scrambled siRNA or anti-Pearl 1 siRNA prior to exposure to the listed stimuli. Lysates were analyzed by immunoblot assays for the indicated proteins.

FIG. 45G is a set of immunoblots. HUVECs were pretreated with DMSO (carrier) or PP1 (SFK inhibitor), prior to exposure to the listed stimuli. Lysates were analyzed by immunoblot assays for the indicated proteins.

FIG. 45H is a set of immunoblots. HUVECs were pretreated with DMSO (carrier) or Dynasore (dynamin inhibitor) prior to exposure to the listed stimuli. Lysates were analyzed by immunoblot assays for the indicated proteins.

FIG. 451 is a graph showing adherence of HUVECs to increased concentrations of SVEP1 or pFN. Wells were precoated with the indicated protein and blocked with BSA prior to assay. Error bars represent 95% confidence intervals.

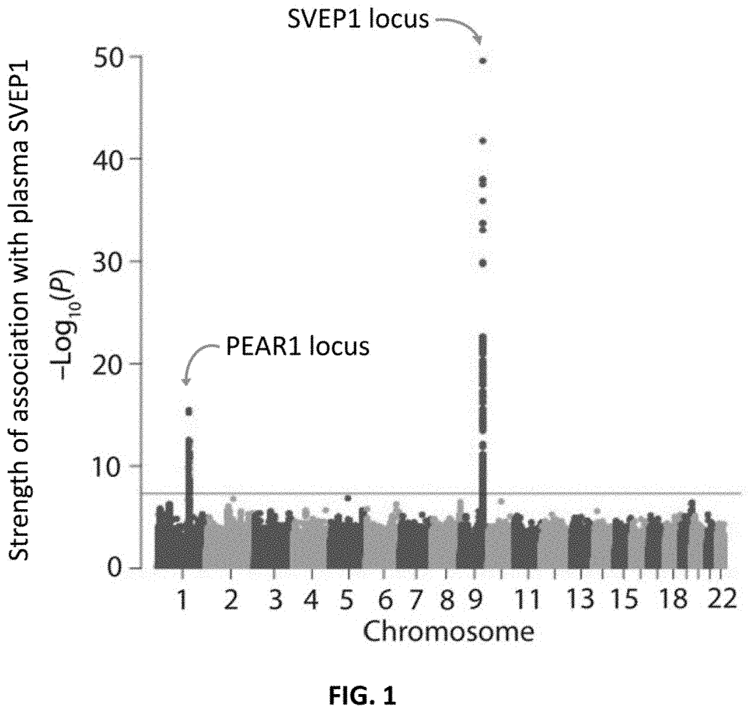

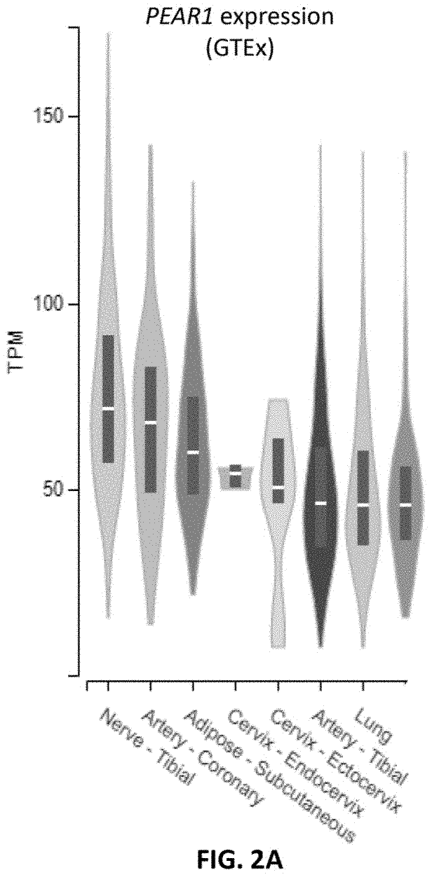

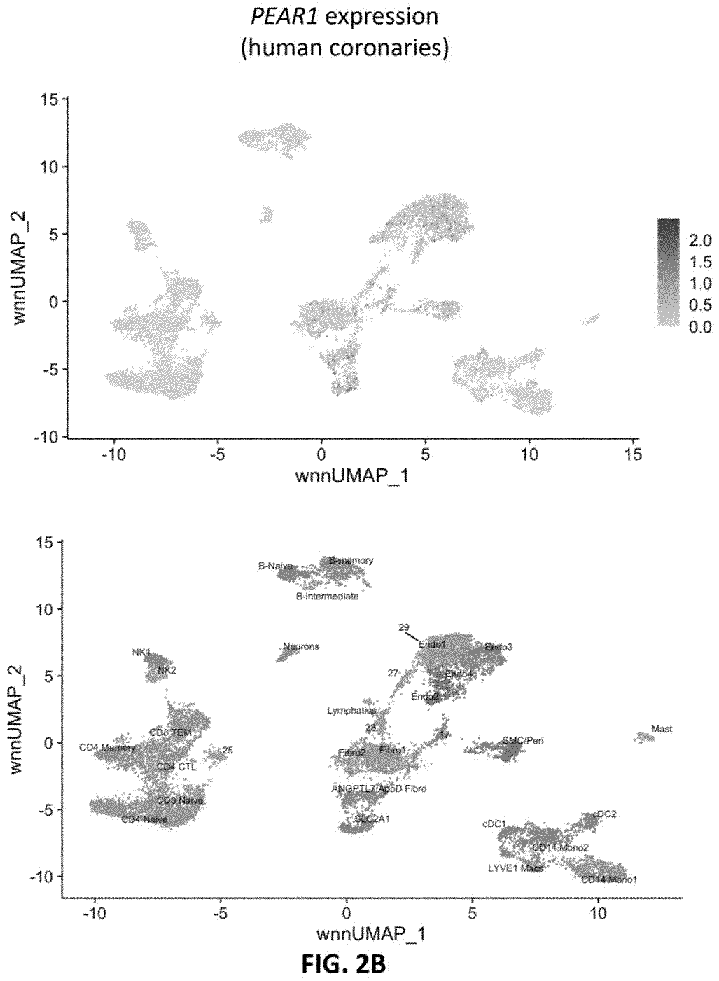

FIG. 46A is a set of images of hCASMCs pre-treated with scrambled siRNA or anti-Pearl 1 siRNA seeded on immobilized SVEP1 for 60 minutes. Scale bar=20 μm. The composite image includes DAPI (teal).

FIG. 46B is a graph of the quantification of Pearl 1 and pAKT colocalization in the images in FIG. 46A, as determined by the Pearson correlation coefficient. Lamellipodia were identified as bundles of fActin on the periphery of cells. Cellular regions not containing lamellipodia were used as control regions. N=22-36, P <0.0001, ANOVA with post hoc unpaired t-test.

FIG. 46C is a set of immunoblots. HUVECs exposed to immobilized BSA or SVEP1, soluble Pearl 1 pAb, or serum for 10 or 30 minutes. Lysates were analyzed by immunoblot assays for the indicated proteins.

FIG. 46D is a set of immunoblots. hCASMCs were transfected with scrambled siRNA or anti-Pearl 1 siRNA prior to exposure to the listed stimuli. Lysates were analyzed by immunoblot assays for the indicated proteins.

FIG. 46E is a set of immunoblots. Platelets were pretreated with DMSO (carrier), PP1 (SFK inhibitor), Dynasore (dynamin inhibitor), MK-2206 (AKT inhibitor), or Rapamycin (mTOR inhibitor) prior to exposure to BSA or SVEP1. Lysates were analyzed by immunoblot assays for the indicated proteins.

FIG. 47A is a graph of platelet counts in whole blood from Svep1+/+ and Svep1 1351 −/− mice. N=8-13.

FIG. 47B is a graph of Mouse platelet receptor density determined by mean fluorescent intensity (MFI) in whole blood from Svep1+/+ and Svep1−/−, as determined by flow cytometry. N=18-21. Bars represent least square means and error bars represent the standard error of difference.

FIG. 47C is a graph of the quantification of mouse activated CD41/61+ platelets, as determined by gating after flow cytometry of isolated platelets in resting conditions, or upon stimulation with ADP or PAR4-AP. N=17-20. Bars represent least square means and error bars represent the standard error of difference.

FIG. 47D is a graph of the quantification of mouse P-selectin+ platelets determined by gating after flow cytometry of isolated platelets in resting conditions, or upon stimulation with ADP or PAR4-AP. N=16-20. Two-way ANOVA. Bars represent least square means and error bars represent the standard error of difference.

FIG. 47E is a graph of the quantification of adherence of platelets to BSA (negative control), SVEP1, or Fibrinogen (positive control) coated coverslips for 10-30 minutes without an agonist or with the addition of 0.1 U/mL thrombin. N=3.

FIG. 47F is a graph of human whole blood platelet counts before and after the addition of SVEP1. N=4. Paired t-test.

FIG. 47G is a graph of the quantification of human platelet receptor density determined by mean fluorescent intensity (1VIFI) of the indicated proteins in freshly isolated platelets as determined by flow cytometry. N=8. Paired t-test.

FIG. 47H is a graph of the quantification of human activated CD41/61+ platelets were determined by gating after flow cytometry of isolated platelets in resting conditions, or upon stimulation with 10 μM ADP, 10 μM TRAP6, or 0.1 U/mL thrombin. N=8. Paired t-test.

FIG. 47I is a graph of the quantification of human P-selectin+ platelets as determined by gating after flow cytometry of isolated platelets in resting conditions, or upon stimulation with 10 μM ADP, 10 μM TRAP6, or 0.1 U/mL thrombin. N=8. Paired t-test.

FIG. 47J is a graph of the proportion of aggregated platelets as determined using murine whole blood cell counts from blood collected from Pear1+/+ and Pear1−/− mice and treated with soluble SVEP1. The proportion of platelets aggregated was calculated as the difference in platelet count after the addition of SVEP1 compared to before, taken as a proportion of the total platelets. N=17-19.

FIG. 47K is a graph of activated CD41/61+ platelets as determined by gating after flow cytometry of isolated platelets before and after SVEP1 addition in resting conditions, or upon stimulation with ADP or PAR4-AP. N=5. Unpaired t-test.

FIG. 47L is a graph of the quantification of P-selectin+ platelets as determined by gating after flow cytometry of isolated platelets before and after SVEP1 addition in resting conditions, or upon stimulation with ADP or PAR4-AP. N=5. Unpaired t-test. *P<0.05, **P<0.01, ***P<0.001.

FIG. 48A is a plot of two-sample MR of estimated SNP effects (with 95% confidence intervals) on MPV (y-axis) and either Pearl 1, green, or SVEP1, blue, in deCODE (x-axis). The causal estimate is designated by a line of the corresponding color. SVEP1 Beta=0.018, P=5.1×10-6; Pearl 1 Beta=0.11, P=1.8×10-8.

FIG. 48B is a plot of two-sample MR of estimated SNP effects (with 95% confidence intervals) on platelet count (y-axis) and either Pearl 1, green, or SVEP1, blue, in deCODE (x-axis). The causal estimate is designated by a line of the corresponding color. SVEP1 Beta=-0.0075, P=0.015; Pearl 1 Beta=-0.048, P=2.3×10-5.

FIG. 48C is a plot of two-sample MR of estimated SNP effects (with 95% confidence intervals) on cardiovascular disease (y-axis) and either Pearl 1, green, or SVEP1, blue, in deCODE (x-axis). The causal estimate is designated by a line of the corresponding color. SVEP1 Beta=0.058, P=4.5×10-12; Pearl 1 Beta=0.067, P=0.0051.

FIG. 48D is a schematic of a model of the vascular wall and lumen. Solid lines represent experimentally tested relationships. Dashed lines represent relationships supported by indirect evidence.

FIG. 49A is a LocusZoom plot of the genetic locus containing Pearl 1 and associations with altered plasma SVEP1 (aptamer 11109.56.3) from the INTERVAL database. Linkage disequilibrium with rs14763900 (purple diamond) is indicated.

FIG. 49B is a plasma SVEP1 (aptamer 11178.21.3) box and whisker plot as a function of allelic copies of rs147639000 (Pearl 1 p.D343N) in the INTERVAL database. Beta=0.70, P=1.7×10-17.

FIG. 50A is a set of immunoblots of the indicated proteins after co-immunoprecipitation. Biotinylated Pearl 1 was detected using a fluorescent streptavidin probe. The control sample did not contain SVEP1.

FIG. 50B is a plot of SVEP1 and Pearl 1 expression in single-cell populations of human coronary arteries from publicly available data.

FIG. 50C is a set of immunoblots of Fibronectin from enriched VSMC media after co-immunoprecipitation of SVEP1 with a-Myc beads.

FIG. 51A is a set of immunoblots. 293T cells were transfected with an empty vector or a Pearl 1-expression plasmid prior to exposure to the listed stimuli. Lysates were analyzed by immunoblot assays for the indicated proteins.

FIG. 51B is a set of immunoblots. hCASMCs were transfected with scrambled siRNA or anti-Pearl 1 siRNA (construct B) prior to exposure to the listed stimuli. Lysates were analyzed by immunoblot assays for the indicated proteins.

FIG. 51C is a set of images of HUVECs seeded on immobilized SVEP1 for 60 minutes. Scale bar=20 μm.

FIG. 51D is a graph of the quantification of Pearl 1 and pAKT colocalization in the images in FIG. 51C, as determined by the Pearson correlation coefficient. Lamellipodia were identified as bundles of fActin on the periphery of cells. Cellular regions not containing lamellipodia were used as control regions. N=28-31, P<0.0001, unpaired t-test.

FIG. 51E is a plot of densitometry quantification of the listed phospho-protein using a phospho-array of HUVEC lysates from cells exposed to immobilized SVEP1 relative to BSA for 10 or 30 minutes.

FIG. 51F is a graph of densitometry quantification of three independent experiments, represented in FIG. 46D. Data are normalized to si-Scrambled, BSA conditions and represent the ratio of phosphop70S6K (Thr 289) to total p70S6K. P<0.01, unpaired t-test.

FIG. 52A is a graph of weekly body mass measurements of Svep1+/+ and Svep1−/− mice after 6 months of HFD feeding. N=12-13. The shaded region represents SEM

FIG. 52B is a graph of the lean mass of Svep1+/+ and Svep1−/− mice after 6 months of HFD feeding, as determined by EchoMRITM. N=10.

FIG. 52C is a graph of the fat mass of Svep1+/+ and Svep1−/− mice after 6 months of HFD feeding, as determined by EchoMRITM. N=10.

FIG. 52D is a graph of the total water of Svep1+/+ and Svep1−/− mice after 6 months of HFD feeding, as determined by EchoMRITM. N=10.

FIG. 52E is a graph of blood glucose measurements of Svep1+/+ and Svep144 −/− mice after intraperitoneal glucose administration at 0 minutes. N=11-13.

FIG. 52F is a graph of blood glucose measurements of Svep1+/+ and Svep1−/− mice after intraperitoneal insulin administration at 0 minutes. N=12-13.

FIG. 52G is a graph of respiratory exchange ratio of Svep1+/+ and Svep1-−/− mice, as determined by indirect calorimetry. N=8. Shaded regions represent 95% confidence intervals.

FIG. 52H is a graph of the metabolic rate (kcal/hr/kg) of Svep1+/+ and Svep1−/− mice, as determined by indirect calorimetry. N=8. Shaded regions represent 95% confidence intervals.

FIG. 53A is a graph of systolic blood pressure of anesthetized Svep1+/+ and Svep1−/− mice, as determined by arterial catheterization.

FIG. 53B is a graph of diastolic blood pressure of anesthetized Svep1+/+ and Svep1−/− mice.

FIG. 53C is a graph of heart rate of anesthetized Svep1+/+ and Svep1−/− mice.

FIG. 53D is a graph of the change in blood pressure upon venous phenylephrine administration in Svep1+/+ and Svep1−/− mice.

FIG. 53E is a graph of the change in blood pressure upon venous angiotensin II administration in Svep1+/+ and Svep1−/− mice.

FIG. 53F is a graph of the change in blood pressure upon venous acetylcholine administration in Svep1+/+ and Svep1−/− mice.

FIG. 53G is a graph of the change in blood pressure upon venous sodium nitroprusside administration in Svep1+/+ and Svep1−/− mice. N=4-5.

FIG. 53H is a graph of aortic compliance of the ex-vivo aortas from Svep1+/+ and Svep1−/− mice as determined by pressure-diameter tracings.

FIG. 53I is a graph of carotid artery compliance of the ex-vivo carotid arteries from Svep1+/+ and Svep1−/− mice as determined by pressure-diameter tracings. N=5 for all panels unless otherwise noted.

FIG. 54A is a graph of platelet receptor density determined by mean fluorescent intensity (MFI) of the indicated proteins in platelets from whole human blood as determined by flow cytometry. N=11. Paired t-test.

FIG. 54B is a graph of the quantification of activated CD41/61+ platelets as determined by gating after flow cytometry of platelets from whole human blood in resting conditions, or upon stimulation with 10 μM ADP, 10 μM TRAP6, or 0.1 U/mL thrombin. N=10-11. Paired t-test.

FIG. 54C is a graph of the quantification of P-selectin+ platelets as determined by gating after flow cytometry of platelets from whole human blood in resting conditions, or upon stimulation with 10 μM ADP, 10 μM TRAP6, or 0.1 U/mL thrombin. N=10-11. Paired t-test.

FIG. 54D is a graph of RBC counts in whole blood isolated from Svep1+/+ and Svep1−/− mice N=20-21.

FIG. 54E is a graph of the white blood cell count of blood isolated from Svep1+/+ and Svep1−/− mice as determined by hemocytometry. N=20-21. *P<0.05, **P<0.01, ***P<0.001, Two-way ANOVA for all panels, unless otherwise noted.

FIG. 54F is a graph of lymphocyte count of blood isolated from Svep1+/+ and Svep1−/− mice as determined by hemocytometry. N=20-21. *P<0.05, **P<0.01, ***P<0.001, Two-way ANOVA for all panels, unless otherwise noted.

FIG. 54G is a graph of the monocyte count of blood isolated from Svep1+/+ and Svep1−/− mice as determined by hemocytometry. N=20-21. *P<0.05, **P<0.01, ***P<0.001, Two-way ANOVA for all panels, unless otherwise noted.

FIG. 54H is a graph of neutrophil count of blood isolated from Svep1+/+ and Svep1−/− mice as determined by hemocytometry. N=20-21. *P<0.05, **P<0.01, ***P<0.001, Two-way ANOVA for all panels, unless otherwise noted.

FIG. 54I is a graph of eosinophil count of blood isolated from Svep1+/+ and Svep1−/− mice as determined by hemocytometry. N=20-21. *P<0.05, **P<0.01, ***P<0.001, Two-way ANOVA for all panels, unless otherwise noted.

FIG. 54J is a graph of basophil count of blood isolated from Svep1+/+ and Svep1−/− mice as determined by hemocytometry. N=20-21. *P<0.05, **P<0.01, ***P<0.001, Two-way ANOVA for all panels, unless otherwise noted.

FIG. 54K is a plot of densitometry quantification of the listed protein using a cytokine array of blood isolated from Svep1+/+ and Svep187 −/− mice. N=4.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the disclosure is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, the compositions and methods of treatment of the present disclosure are based on the discovery that SVEP1 signaling is mediated by Pearl 1. SVEP1 binds to Pearl 1, an orphan receptor tyrosine kinase-like protein and activates the AKT/mTOR pathway in endothelial cells. Given the role of AKT/mTOR signaling in disease and aging, this interaction is thought to underlie SVEP1's association with vascular diseases of aging in humans.

SVEP1 is a large extracellular matrix protein expressed by fibroblasts and vascular smooth muscle cells (VSMCs) of human and mouse artery walls. SVEP1 governs cell proliferation and differentiation and is more induced in diseased aortas under atherosclerotic conditions. SVEP1 is causally related to longevity and several vascular-related human diseases.

Pearl 1 (Platelet Endothelial Aggregation Receptor 1) is an orphan receptor with no known biological ligand, despite extensive study. Pearl 1 is expressed in endothelial cells, VSMCs, and fibroblasts of coronary arteries. Pearl 1 is a tyrosine kinase-like receptor that is phosphorylated by Src family kinases (SFK). Activation of Pearl 1 results in robust activation of pAKT activation, including, but not limited to, activation of the AKT/mTOR pathway.

As described herein, SVEP1 and Pearl 1 variants have similar human disease associations. Non-limiting examples of human diseases associated with both SVEP1 and Pearl 1 variants include coronary artery disease, elevated systolic and diastolic blood pressure, platelet reactivity, and platelet count.

As described in the examples below, human plasma proteomics methods were used to generate hypotheses about SVEP1's disease mechanisms. These hypotheses were tested using molecular and cellular approaches, such as protein pull-down assays, cell signaling experiments, and endothelial cell behavior assays. The results of protein pull-down assays indicated that SVEP1 and Pearl 1 co-immunoprecipitated with each other, consistent with a direct physical interaction between the two proteins. In various cell signaling experiments, endothelial cells exposed to SVEP1 have an increase in phosphorylation of Pearl 1, the first step in Pearl 1 signaling, suggesting SVEP1 signals through Pearl 1. In addition, endothelial cells (HUVECs) and vascular smooth muscle cells (hCASMCs) express Pearl 1 and AKT is phosphorylated when these cells are exposed to SVEP1. AKT phosphorylation is downstream from Pearl 1 phosphorylation. 293T cells do not express Pearl 1 and do not activate AKT signaling in response to SVEP1. SVEP1 also induces AKT signaling in human platelets which express high levels of Pearl 1, consistent with Pearl 1 activation in these cells when exposed to SVEP1.

In addition, Src is necessary for SVEP1-induced activation of AKT signaling, consistent with Pearl 1-associated activation and signaling. Endocytosis is necessary for SVEP1-induced activation of AKT signaling, also consistent with Pearl 1 activation and signaling.

As described in the examples below, Pearl 1 was shown to activate the mTOR pathway. mTOR is activated 30 minutes after exposure to SVEP1. mTOR pathway activation has not been described before for Pearl 1, and no signaling beyond AKT activation has been shown for Pearl 1 activation.

As further described in the examples below, SVEP1 causes platelet pre-activation and clumping, and platelets from Svep1knockout mice are less “pre-activated” than control mice and respond less to agonists ADP and PAR4.

Thus, inhibiting SVEP1 and/or inhibiting the binding of SVEP1 with Pearl 1 represents a novel non-lipid therapeutic approach for the prevention or treatment of coronary artery disease.

### I. SVEP1-Mediated Disorders

In various aspects, the compositions and methods of treatment disclosed herein are configured to disrupt SVEP1-Pearl 1 signaling induced by the binding of SVEP1 to Pearl 1 receptors expressed in various cells including, but not limited to, endothelial cells, smooth muscle cells, platelets, and fibroblasts. Non-limiting examples of specific cell types found to express Pearl 1 as described in the examples below include vascular endothelial cells such as human umbilical vein endothelial cells (HUVECs), vascular smooth muscle cells (VSMCs), fibroblasts of coronary arteries, and primary human coronary artery smooth muscle cells (hCASMCs).

As described in the examples below and as illustrated in FIG. 48D, the binding of SVEP1 activates canonical Pearl 1 signaling in a Pearl 1-dependent fashion. Specifically, SVEP1 binding to a Pearl 1 receptor induces AKT activation and associated downstream mTOR signaling. Without being limited to any particular theory, SVEP1/Pearl 1 signaling and associated signaling pathways are thought to directly or indirectly contribute to platelet biology, cardiometabolic disease, and longevity.

In various other aspects, the compositions and methods of treatment are configured to disrupt SVEP1-modulated processes associated with the development of atherosclerosis and metabolic disorders, as described in additional detail below.

SVEP1 is mainly expressed in VSMCs in both human and mouse arteries, and SVEP1 is induced during the development of atherosclerosis. By way of non-limiting example, Apoe−/− mice with whole-body knockout of SVEP1 that were fed a high-fat diet for 8 weeks showed decreased atherosclerosis relative to Apoe−/− mice lacking the knockout.

Without being limited to any particular theory, atherosclerosis is thought to be a dysregulated or maladaptive process of provisional matrix formation within arteries. Provisional matrix formation may be initiated by a focal inflammation of an artery that results in the release of matrix remodeling enzymes by macrophages and activated VSMCs within the artery wall. The matrix remodeling proteins may degrade endothelial basement membrane and arterial matrix proteins. VSMCs may infiltrate the intima and produce a provisional matrix that may include fibronectin, tenascin C, osteopontin, and SVEP1.

### II. Molecular Engineering

The following definitions and methods are provided to better define the present disclosure and to guide those of ordinary skill in the art in the practice of the present disclosure. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit the translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule, which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities, and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. Based on this artificially generated polypeptide sequence, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6× SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6× SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C. +16.6(log10 [Na+]) +0.41(fracti on G/C content)−0.63(% formamide)−(600/1). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microproj ectile-mediated delivery, receptor-mediated uptake, cell fusion, electroporation, and the like. The transfected cells can be selected and propagated to provide recombinant host cells that comprise the expression vector stably integrated into the host cell genome.

Exemplary nucleic acids that may be introduced to a host cell include, for example, DNA sequences or genes from another species, or even genes or sequences that originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, Tex.; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc., Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption-delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes that include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating or preventing vascular diseases and/or thrombosis in a subject in need administration of a therapeutically effective amount of a Pearl 1 antagonist compound, so as to reduce expression and/or availability of Pearl 1 in the subject in need.

In one aspect, the Pearl 1 antagonist compound may bind or complex with SVEP1 produced by cells within the subject so as to render the Pearl 1 biologically inactive. In another aspect, the Pearl 1 antagonist compound may bind or complex with Pearl 1, a receptor with affinity for SVEP1 ligand, so as to disrupt SVEP1 binding.

Methods described herein are generally performed on a subject in need thereof. A subject in need of the therapeutic methods described herein can be a subject having, diagnosed with, suspected of having, or at risk for developing a vascular disease and/or thrombosis. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of the Pearl 1 antagonist compound is, for example, that amount that would cause the desired therapeutic effect in a subject while minimizing undesired side effects. In various embodiments, an effective amount of the Pearl 1 antagonist compound described herein can substantially inhibit vascular disease and/or thrombosis, slow the progress of vascular disease and/or thrombosis, or limit the development of vascular disease and/or thrombosis.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of the Pearl 1 antagonist compound can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to prevent or limit the development of vascular disease and/or thrombosis.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shanqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the Pearl 1 antagonist compound can occur as a single event or over a time course of treatment. For example, the Pearl 1 antagonist compound can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for vascular disease and/or thrombosis.

The Pearl 1 antagonist compound can be administered simultaneously or sequentially with another agent, such as an antibiotic, an anti-inflammatory, or another agent. For example, the Pearl 1 antagonist compound can be administered simultaneously with another agent, such as an antibiotic or an anti-inflammatory. Simultaneous administration can occur through administration of separate compositions, each containing one or more of the Pearl 1 antagonist compound, an antibiotic, an anti-inflammatory, or another agent. Simultaneous administration can occur through administration of one composition containing two or more of the Pearl 1 antagonist compound, an antibiotic, an anti-inflammatory, or another agent. The Pearl 1 antagonist compound can be administered sequentially with an antibiotic, an anti-inflammatory, or another agent. For example, the Pearl 1 antagonist compound can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### III. Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of fives” state that a candidate drug-like compound should have at least three of the following characteristics: (i) weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and 0 atoms). In addition, drug-like molecules typically have a span (breadth) of between about 8 Å to about 15 Å.

### IV. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to the Pearl 1 antagonist compound. Such packaging of the components separately can, if desired, be presented in a pack or dispenser device, which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, or sterile saline, each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet website specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Green and Sambrook 2012 Molecular Cloning: A Laboratory Manual, 4th ed., Cold Spring Harbor Laboratory Press, ISBN-10: 1605500569; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following examples illustrate various aspects of the disclosure.

### Example 1: SVEP1 Proteomics

A causal association was previously identified between SVEP1, a large extracellular matrix protein with numerous domains, and cardiovascular disease using a combination of genomic and proteomic approaches. SVEP1 is also associated with human longevity, yet the mechanisms by which this protein influences disease remained uncharacterized.

Human plasma proteomics was used to generate hypotheses about SVEP1's disease mechanisms. These hypotheses were tested using molecular and cellular approaches, such as protein pull-down assays, cell signaling experiments, and endothelial cell behavior assays.

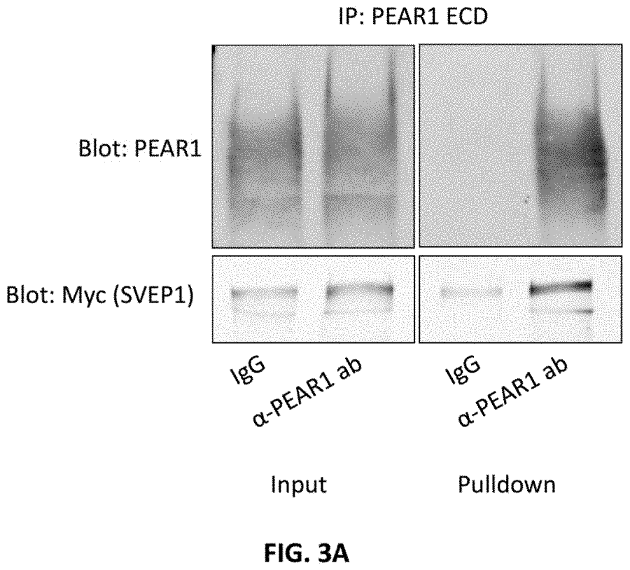

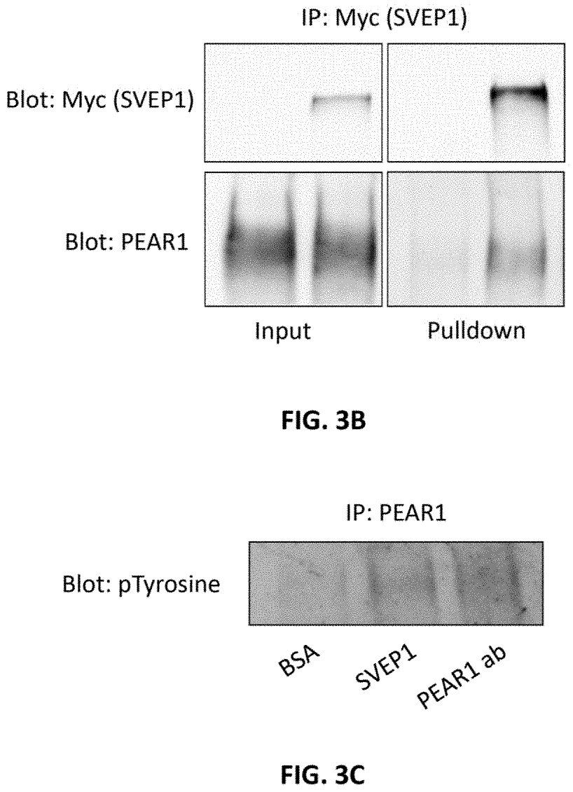

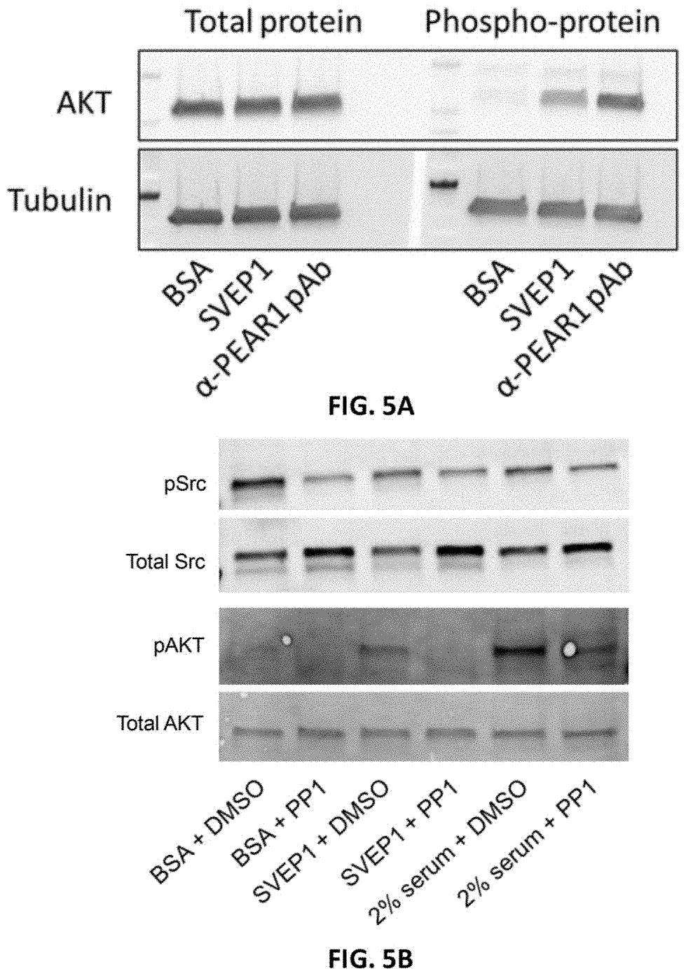

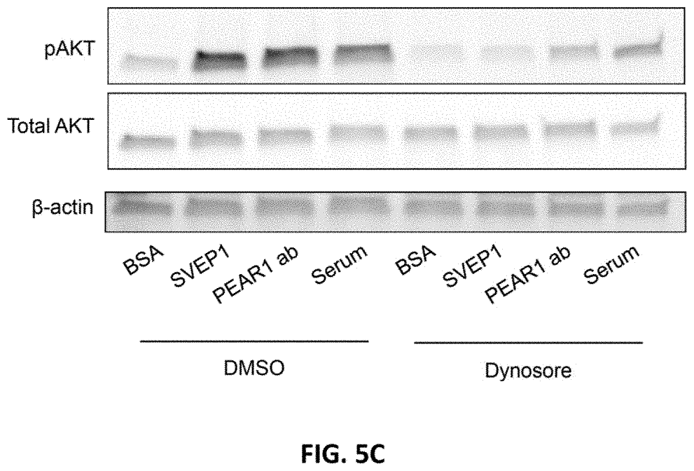

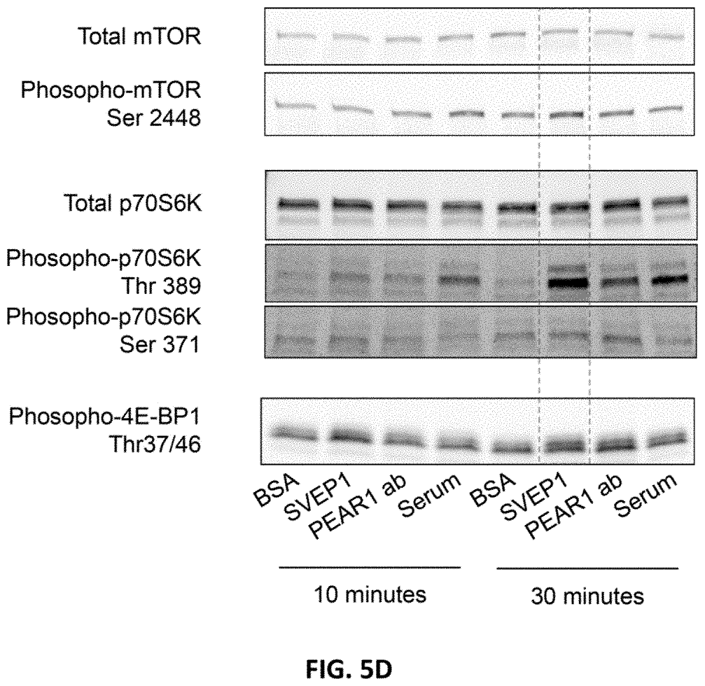

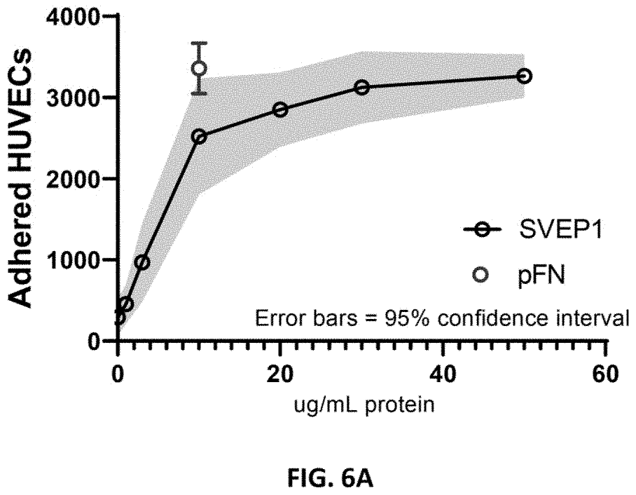

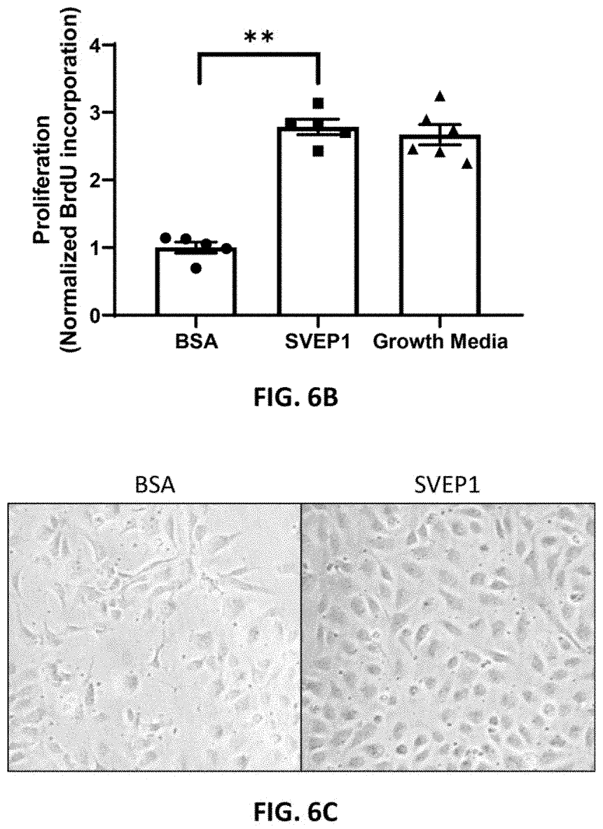

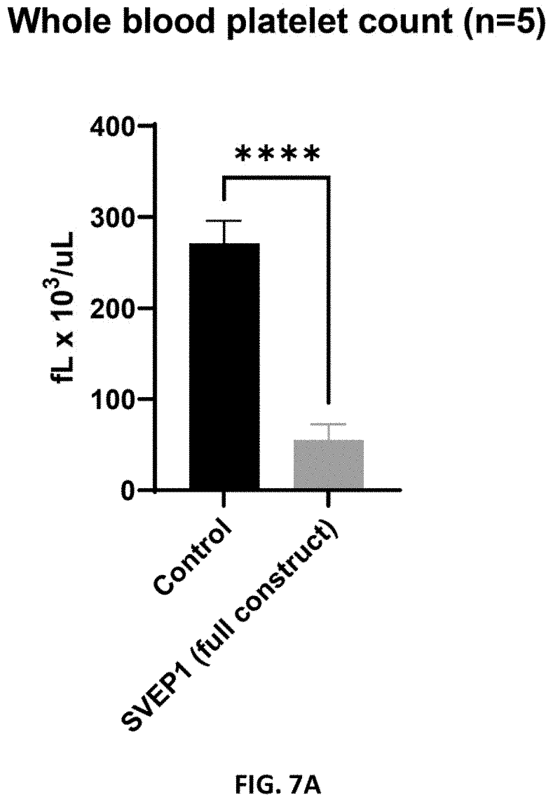

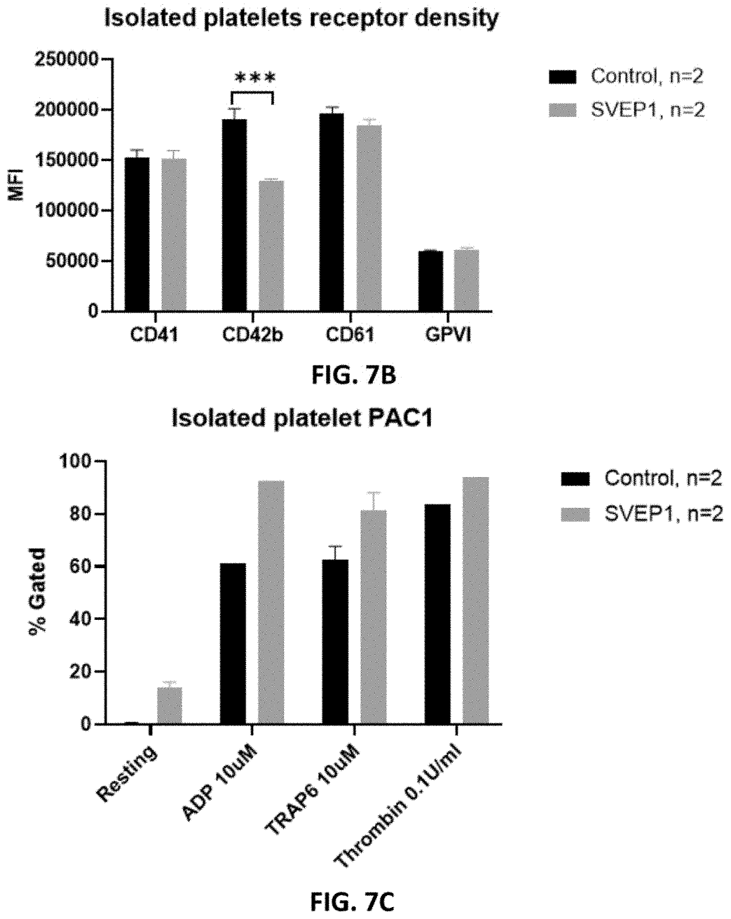

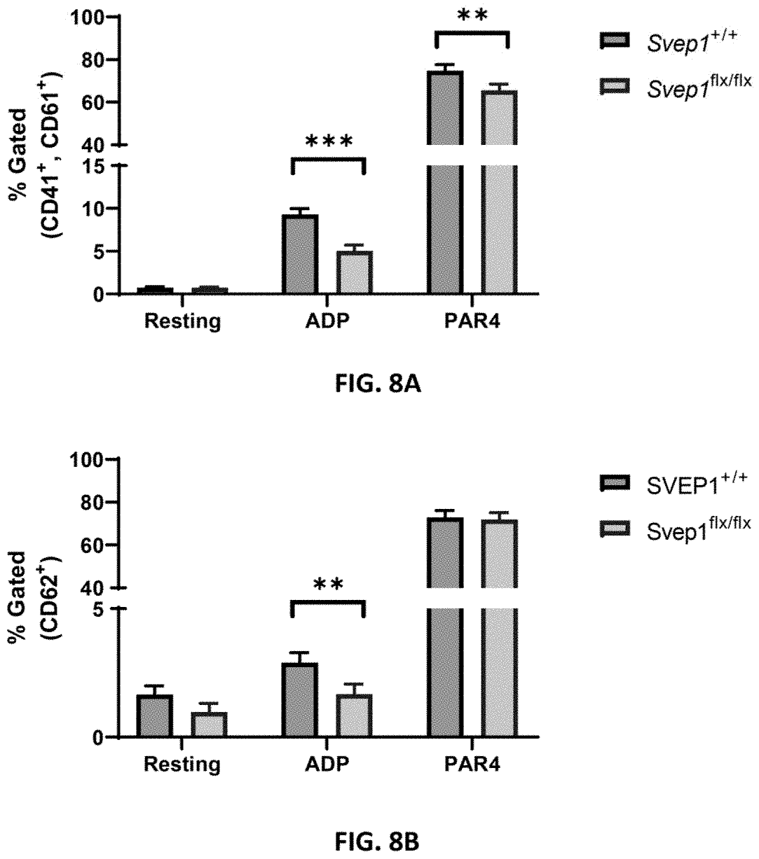

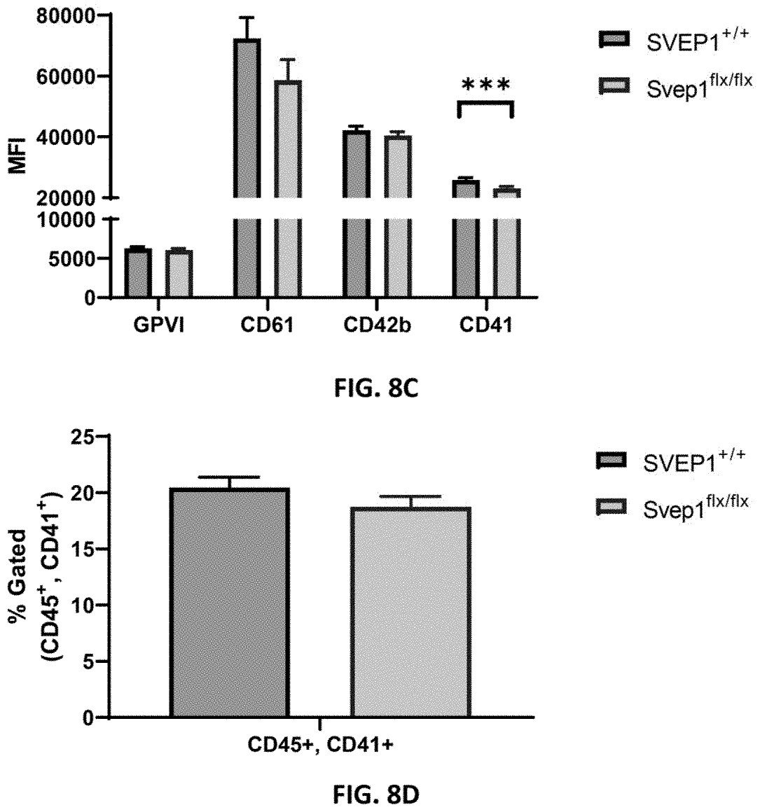

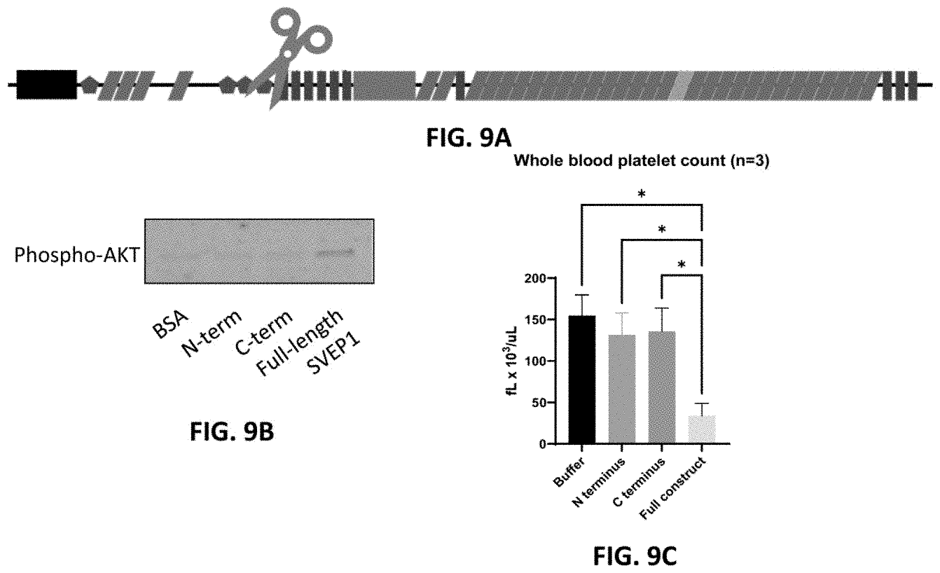

Plasma levels of SVEP1 were influenced by Pearl 1, a protein variant in an orphan receptor tyrosine kinase-like protein. This finding suggested that SVEP1 and Pearl 1 interact. Indeed, in-vitro pull-down assays supported a physical interaction between the two proteins (FIG. 3A 3B, and 3C). SVEP1 modified endothelial cell behavior and also induced activation of AKT/mTOR (FIG. 5D), a signaling pathway related to Pearl 1 activation and aging, further supporting the interaction between SVEP1 and Pearl 1.

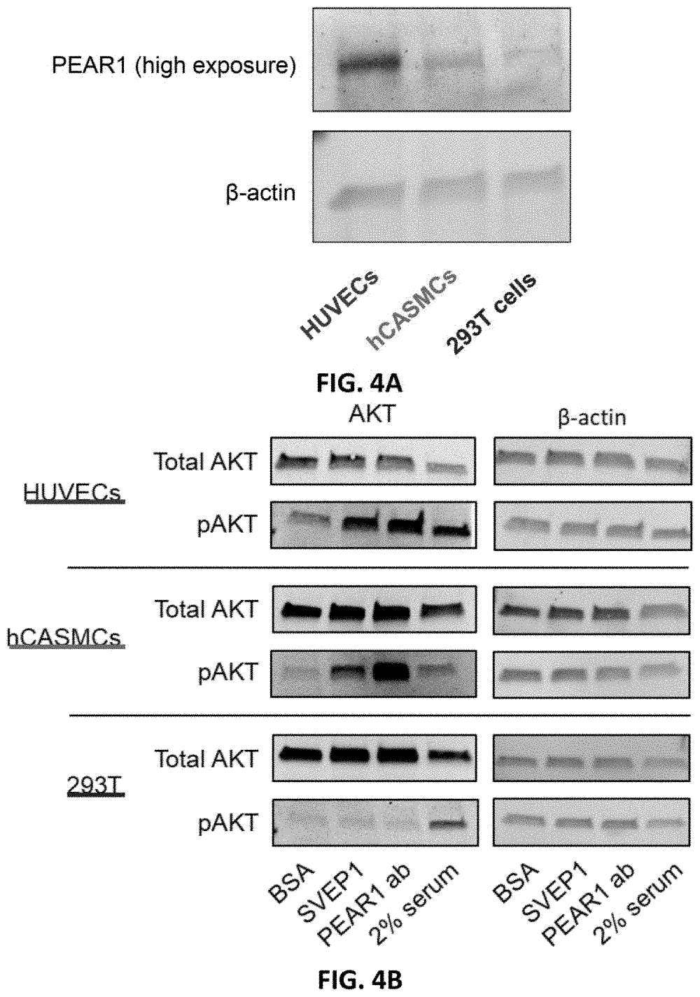

The results of these experiments determined that SVEP1 binds to Pearl 1 (FIG. 3), an orphan receptor tyrosine kinase-like protein, and activated the AKT/mTOR pathway in endothelial cells (FIGS. 4 and 5). Given the role of AKT/mTOR signaling in disease and aging, this interaction likely underlies SVEP1's association with vascular diseases of aging in humans.

### Methods

The following examples make use of at least one or more of the methods described below.

### a) Mice

SVEP1^{+/-} mice were obtained and crossed with mice expressing the flippase FLP recombinase under the control of the promoter of the human actin beta gene (hATCB) to generate SVEP1^{flox/flox} (SVEP1^{fl/fl}) mice. CRISPR/Cas9 genome editing technology was used to generate SVEP1^{mut/mut} (aka SVEP^{G/G}) mice harboring the SVEP1 mutation at the homologous murine position (p.D2699G). SVEP1^{+/-} and SVEP1^{mut/mut} mice were crossed with Apoe^{-/-} mice (Jackson Laboratory, #002052) to get SVEP1^{+/-}Apoe^{-/-} and SVEP^{mut/+}Apoe^{-/-} mice, which were maintained as breeders to generate experimental and control mice. SVEP1^{fl/fl} mice were crossed with Myh11-CreER^{T2} (Jackson Laboratory, #019079) mice to generate SVEP1^{fl/+}Myh11-CreER^{T2} mice. SVEP1^{fl/+}Myh11-CreER^{T2} males were crossed with SVEP1^{fl/+} females to generate experimental SVEP1^{fl/fl}Myh11-CreER^{T2} and control SVEP1^{30 /+}Myh11-CreER^{T2} male littermate mice. SVEP1^{fl/fl}Myh11-CreER^{T2} males were crossed with Apoe^{-/-} females. SVEP1^{fl/+}Myh11-CreER^{T2}Apoe^{-/-} males and SVEP1^{fl/+}Apoe^{-/-} females were maintained as breeders to generate experimental and control mice.

To activate Cre-recombinase, mice were injected intraperitoneally with 1 mg of tamoxifen (#T5648, Sigma-Aldrich) in 100 μL peanut oil (#P2144, Sigma-Aldrich) for 10 consecutive days starting at 6 weeks of age. Tamoxifen treatment was performed with all experimental and control mice in an identical manner. Itgα9^{flox/flox} (Itgα9^{fl/fl} ) mice and LysM-Cre mice were obtained from other researchers. Itgα9^{fl/fl} mice were crossed with LysM-Cre mice to generate Itgα9^{fl/fl}LysM-Cre (Itgα9^{MACfl/fl}) and control Itgα9 ^{+/+}LysM-Cre (Itgα9^{MAC+/+}) mice. All mice were housed in separate cages in a pathogen-free environment and maintained on a 12 hr. light/12 hr. dark cycle with a room temperature of 22±1° C.

### b) Diet and Assessment of Atherosclerosis

All experimental mice were fed a high-fat diet containing 21% fat and 0.2% cholesterol (#TD.88137, Envigo Teklad) for 8 weeks starting at 8 weeks of age. After 8 weeks of high-fat diet feeding, blood was collected from the retro-orbital plexus after 12 hr. fasting, and mice were euthanized by carbon dioxide inhalation. Plasma samples were prepared from the collected blood by centrifugation at 13,000 rpm for 10 min at 4° C. Total cholesterol (#STA-384), triglycerides (#STA-397), and glucose (#STA-681) in mouse plasma were determined using each kit (all purchased from Cell Biolabs, Inc.). Hearts and whole aortas (from the aortic arch to the iliac artery) were harvested after perfusion with phosphate-buffered saline (PBS). For en face analysis, isolated aortas were cleaned by removal of perivascular fat tissues, opened longitudinally, and pinned onto black wax plates. After fixation with 4% paraformaldehyde overnight at 4° C., aortas were washed with PBS for 1 hr. and stained with 0.5% Oil Red O in propylene glycol (#01516, Sigma-Aldrich) for 3 hr. at room temperature. After staining, aortas were de-stained with 85% propylene glycol in distilled water for 5 min to reduce background staining and washed with distilled water for 15 min. For analysis of plaque in the aortic root, hearts were fixed overnight with 4% paraformaldehyde at 4° C., washed with PBS for 1 hr., and embedded into OCT compound (#4583, Sakura® Finetek). 5-μm-thick cryosections were stained overnight with 0.5% Oil Red O in propylene glycol, de-stained with 85% propylene glycol in distilled water for 5 min, and washed with distilled water for 15 min. Measurement of plaque was performed using 6-8 sections per artery to get the average value of size. The atherosclerotic plaque area was digitized and calculated using AxioVison (Carl Zeiss).

### c) Antibodies and Reagents

For immunofluorescent staining, anti-β-galactosidase (#ab9361, abcam, 1:1000), anti-Mac-3 (#550292, clone M3/84, BD Biosciences, 1:100), anti-SMα-actin-cy3 (#C6198, clone 1A4, Sigma-Aldrich, 1:1000), anti-MCM-2 (#4007, Cell Signaling, 1:100) were used, and then visualized with anti-chicken Alexa 488 (#A1103, Invitrogen 9), anti-rat Alexa488 (#A21470, Invitrogen), anti-rat Alexa594 (#A21471, Invitrogen), and ProLone^{m} Gold antifade reagent with DAPI (#P36935, Invitrogen). For detection of MCM-2 staining, samples were visualized with anti-rabbit-HRP (#7074S, Cell Signaling, 1:1000) followed by TSA® Plus Cyanine 5 (#NEL745E001KT, PerkinElmer).

For immunohistochemistry studies, hematoxylin solution (#HHS80, Sigma-Aldrich), eosin solution (#HT110180, Sigma-Aldrich), Masson's trichrome staining kit (#HT15-1KT, Sigma-Aldrich), and Permount solution (#SP15-500, Fisher Chemicals) were used. For flow cytometry, the following anti-mouse antibodies were used; anti-CD16/32 FcR blocker (#14-0161, eBioscience), PerCP-labeled anti-CD45 (#103129, clone 30-F11), BV510-labeled anti-CD11b (#101263, clone M1/70), BV421-labeled anti-CD64 (#139309, clone X54-5/7.1), PE/cy7-labeled anti-CD11 c (#117317, clone N418), APC/cy7-labeled anti-MHCII (#107627, clone M5/114.15.2), FITC-labeled anti-F4/80 (#123108, clone BM8), BV605-labeled anti-CD19 (#115540, clone 6D5), APC-labeled anti-CD115 (#135510, clone AFS98), Alexa700-labeled anti-Ly6C (#128023, clone HK1.4), PE/Cy7-labeled anti-Ly6G (#127618, clone 1A8, all purchased from Biolegend), PE/cy5.5-labeled anti-CD4 (#35-0042-82, clone RM4-5, eBioscience), Alexa700-labeled anti-CD8a (#56-0081-80, clone 53-6.7, eBioscience), and PE-labeled anti-Itg α9β1 (#FAB3827P, R&D systems). In addition, the following anti-human antibodies were used: FcR blocker (#564219, BD biosciences), PerCP/cy5.5-labeled anti-CD45 (#368504, clone 2D1), Alexa700-labeled anti-CD3 (#300323, clone HIT3a), anti-CD19 (#115527, clone 6D5), anti-CD56 (#392417, clone QA17A16), FITC-labeled anti-CD15 (#301904, clone HI98), BV421-labeled CD66b (#305111, clone G10F5), APC/cy7-labeled anti-CD14 (#325619, clone HCD14), BV605-labeled anti-CD16 (#360727, clone B73.1), and PE-labeled anti-Itg a9(31 (#351606, clone Y9A2, all purchased from Biolegend).

RBC lysis buffer (#423101, Biolegend), FoxP3 transcription factor staining buffer set (#00-5523, eBioscience), and Leuko spin medium (#60-00091, Pluriselect) were used in flow cytometry experiments.

### d) Immunohistochemistry and Immunofluorescent Staining

All immunohistochemistry and immunofluorescence studies described below used 4% paraformaldehyde-fixed frozen sections with 5-μm-thickness. For immunofluorescent staining, slides were air-dried for 1 hr. at room temperature and hydrated with PBS for 10 min. After permeabilization with 0.5% tritonX-100 for 10 min, sections were blocked with PBS containing 5% chicken serum (#S-3000, Vector Laboratories) with 0.5% tritonX-100 for 1 hr. at room temperature. Blocked sections were incubated with the antibodies indicated above.

For hematoxylin and eosin (H&E) staining, air-dried slides were hydrated in PBS for 10 min, placed in hematoxylin solution for 10 min, and then rinsed in running tap water. After de-staining in 1% acetic acid for 5 min, slides were rinsed in tap water and placed in 90% ethanol for 5 min. Slides were stained with eosin solution for 8 min, gradually dehydrated in ethanol solution (from 80% to 100%), and then incubated with xylene for 10 min followed by mounting with Permount solution.

For Masson's trichrome staining, air-dried slides were hydrated in distilled water for 10 min, placed in Mordant in Bousin's solution for 1 hour at 56° C., and rinsed in running tap water for 5 min. After staining in hematoxylin solution for 10 min, slides were washed in running tap water for 10 min, rinsed in distilled water, placed in Biebrich scarlet-acid fuchsin solution for 15 min, and stained in aniline blue solution for 10 min. After rinsing in distilled water, slides were differentiated in 1% acetic acid for 3 min, gradually dehydrated in ethanol solution (from 80% to 100%), and incubated with xylene for 10 min followed by mounting with Permount solution.

### e) RNAscope In Situ Hybridization (ISH)

For the detection of SVEP1 and Itgα9 RNA transcripts in both human and mouse artery tissues, a commercially available kit (#323100, RNAscope® Multiplex Fluorescent Reagent Kit v2, Advanced Cell Diagnostics) was used according to the manufacturer's instructions. Briefly, 4% paraformaldehyde-fixed mouse tissue samples (including aortic root, aortic arch, BCA), human aortic wall tissue samples, and LIMA frozen sections with 5-μm-thickness were air-dried for 1 hr. at room temperature, and treated with hydrogen peroxide for 10 min to block endogenous peroxidase activity. After antigen retrieval with boiling in target antigen retrieval solution for 5 min at 95-100° C., slides were treated with protease III for 30 min at 40° C. Target probes (#NM_022814.2, mouse SVEP1, 20 pairs, target nucleotide 2879-3726 or NM_001113514.1, mouse Itgα9 , 20 pairs, target nucleotide 389-1365) were hybridized for 2 hr. at 40° C., followed by a series of signal amplification and washing steps. Hybridization signals were detected by TSA® Plus Cyanine 5, and co-stained with indicated antibodies. Slides were counterstained with DAPI by using ProLong™ Gold antifade reagent.

### f) Flow Cytometry

For labeling mice blood cells, blood was collected from the mice's retro-orbital plexus as described above, and red blood cells were removed using RBC lysis buffer. For labeling human blood cells, Leuko spin medium was used to isolate leukocytes from peripheral blood and buffy coat. In the experiment using the mouse spleen described below, spleen cells were recovered from mice by cutting the spleen into small fragments, and incubating the fragments with 400 U collagenase D (#11-088-858, Roche applied science) for 30 min at 37° C. For labeling aortic single-cell suspensions, isolated aortas were perfused with DPBS, and opened longitudinally. The whole artery was cut into 2-5 mm pieces, and the pieces were incubated in an HBSS solution with calcium and magnesium containing 90 U/mL DNase I (#DN25), 675 U/mL collagenase I (#C0130), 187.5 U/mL collagenase XI (#C7657), and 90 U/mL hyaluronidase (#H1115000, all purchased from Sigma-Aldrich) for 70 min at 37° C. with gentle shaking. Non-specific binding to Fc receptors was blocked, and cells were incubated with the indicated antibodies for 30 min at 4° C. For intracellular staining, cells were fixed/permeabilized with the FoxP3 transcription factor staining buffer set. Flow cytometric analyses were performed using the LSRFortessa™ instrument (BD Biosciences) and FlowJo software (Tree Star Inc.).

g) Bead Labeling of Ly6C^{low} Monocytes Recruited into Atherosclerotic Plaque

After 8 weeks of high-fat diet feeding, 200 μL of 1 μm Fluoresbrite yellow-green (YG) microspheres beads (#17154-10, Polysciences, Inc.) diluted 1:4 in sterile DPBS were administered retro-orbitally to the mice. The labeling efficiency of blood monocytes was verified by flow cytometry 3 days after the YG bead injection. Recruitment of YG bead-positive monocytes into plaque in the aortic root was analyzed 1 day after checking the labeling efficiency of YG beads. 5-μm-thick frozen sections of aortic root were stained with anti-Mac-3 antibody, followed by anti-rat Alexa594 antibody. Slides were mounted with ProLong™ Gold Antifade Mountant with DAPI. The number of YG-beads colocalized with Mac-3 positive area was counted and/or normalized with the percentages of labeled Ly6C^{low} monocytes.

### h) Aortic VSMC Culture

Mouse aortic VSMCs were isolated from 8-week-old Apoe^{-/-} and SVER1^{+/-}Apoe^{-/-}, or similarly-aged SVEP1^{SMC+/+} and SVEP1^{SMC fl/fl} mice after tamoxifen injection for 10 consecutive days starting at 6 weeks of age. Briefly, thoracic aortas were harvested (three mice per group were used) and perivascular fat was removed. The aortas were digested in 1 mg/ml collagenase II (#LS004174), 0.744 units/ml elastase (#LS002279), 1 mg/ml soybean trypsin inhibitor (#LS003570, all purchased from Worthington Biochemical Corporation), and 1% penicillin/streptomycin in Hanks' Balanced Salt Solution for 10 min at 37° C. with gentle shaking. After a pre-digestion with enzyme mixture, the adventitial layer was removed under the dissection microscope, and the intimal layer was removed by scrapping with forceps. The aortas were cut into small pieces, and the pieces were completely digested in an enzyme mixture at 37° C. for 1 hr. with gentle shaking. VSMCs were grown in 20% fetal bovine serum (FBS, Hyclone) containing DMEM/F12 media (Gibco) with 100 U/ml penicillin/streptomycin at 37° C., 5% CO2 incubator. After two passages, VSMCs were changed to 10% serum. To stimulate VSMCs for study, human medium oxidized low-density lipoprotein (oxLDL, #770202-7, Kalen biomedical) was used.

### i) Quantitative Real-Time PCR

Gene expression was quantified in the experiments described below using quantitative real-time PCR. RNA was isolated using RNeasy® Mini Kit (#74106, Qiagen) according to the manufacturer's protocol, and QlAshredder homogenizer (#79656, Qiagen) was used to increase the yield of quantification of RNA. cDNA was synthesized with High Capacity cDNA Reverse Transcript Kit (#4368814). Real-time PCR was performed using both Taqman® (#4444557) and SYBR™ Green (#A25742, all purchased from Applied Biosystems) assays. Ct values were normalized to β-actin (for Taqman®) and gapdh (for SYBR™ Green), and normalized relative to control.

j) In Vitro Migration Assay using Peritoneal Macrophage

Itgα9^{Mac+/+} and Itgα9^{MACfl/fl} mice were injected intraperitoneally with 1 ml 4% thioglycolate. After 5 days, the peritoneal cells were collected by lavage and placed in RPMI media containing 10% FBS for 60 min at 37° C. Non-adherent cells were removed after washing with PBS three times, and adherent cells (more than 90% were peritoneal macrophages confirmed by flow cytometry) were placed in Trans-well inserts with a 5 μm porous membrane in a modified Boyden chamber. RPMI media containing 10% FBS with 50 or 200 ng/mL SVEP1 protein was placed in the lower chamber. After allowing cell migration over 16 hr., the inserts were removed from the upper side of the chamber, and the nuclei of cells migrated to the lower side of the membrane were stained with DAPI.

### k) Western Blotting Assay

The Western blotting assay was performed using conditioned media from Apoe^{-/-} and SVEP1^{+/-}Apoe^{-/-} VSMCs cultured with 50 μg/mL oxLDL containing serum-free DMEM/F12 media for 24 and 48 hr. Collected conditioned media was concentrated using Vivaspin® 500 Centrifugal Concentrator following the manufacturer's instruction. Anti-SVEP1 polyclonal antibody (#NBP1-90791, Novus Biologicals) was used.

l) Proliferation Assay

Wells of a 96-well plate were pre-coated with 30 μg/mL recombinant SVEP1 protein or bovine serum albumin (BSA, as an inert protein control). Wells were subsequently blocked with 10 mg/mL BSA and washed twice with DPBS. Plates were UV sterilized before adding cells. SVEP1^{SMC fl/fl} VSMCs were collected and suspended in DMEM/F12 media containing 10% FBS. 2×10³ cells were added to each well and incubated for 8 hr. to assure complete cell adhesion. Media was then replaced with DMEM/F12 media containing 0.2% BSA and incubated for 12 hours to decrease basal proliferation rates. Cells were then incubated in BrdU dissolved in DMEM/F12 media containing 0.2% BSA for 30 hours. For analysis of peritoneal macrophages, 4% thioglycolate-elicited peritoneal macrophages from Itg α9^{MAC+/+} and Itg α9^{MACfl/fl} mice were suspended in BrdU dissolved in RPMI 1640 media containing 10% FBS. 2.5×10⁴ cells were added to each well since peritoneal macrophages are smaller and have lower proliferation rates than VSMCs in culture. 50 μg/mL oxLDL was added to the indicated cells at the beginning of this incubation. An ELISA to quantify incorporated BrdU was then performed as per kit instructions (#6813, Cell Signaling Technologies) after incubation for 36 hr.

### m) Bulk RNA Sequencing and Analysis

Samples were prepared according to the library kit manufacturer's protocol, indexed, pooled, and sequenced on an Illumina HiSeq. Basecalls and demultiplexing were performed with Illumina's bcl2fastq software and a custom python demultiplexing program with a maximum of one mismatch in the indexing read. RNA-seq reads were then aligned to the Ensembl release 76 primary assembly with STAR version 2.5.1a. Gene counts were derived from the number of uniquely aligned unambiguous reads by Subread:featureCount version 1.4.6-p5. Isoform expression of known Ensembl transcripts was estimated with Salmon version 0.8.2. Sequencing performance was assessed for the total number of aligned reads, the total number of uniquely aligned reads, and the features detected. The ribosomal fraction, known junction saturation, and read distribution over known gene models were quantified with RSeQC version 2.6.2. All gene counts were then imported into the R/Bioconductor package EdgeR and TMM normalization size factors were calculated to adjust for samples for differences in library size. Ribosomal genes and genes not expressed in the smallest group size minus one sample greater than one count-per-million were excluded from further analysis. The TMM size factors and the matrix of counts were then imported into the R/Bioconductor package Limma. Weighted likelihoods based on the observed mean-variance relationship of every gene and sample were then calculated for all samples with the voomWithQualityWeights. The performance of all genes was assessed with plots of the residual standard deviation of every gene to their average log-count with a robustly fitted trend line of the residuals. Differential expression analysis was then performed to analyze for differences between conditions and the results were filtered for only those genes with Benjamini-Hochberg false-discovery rate adjusted p-values less than or equal to 0.05. For each contrast extracted with Limma, global perturbations in known Gene Ontology (GO) terms, MSigDb, and KEGG pathways were detected using the R/Bioconductor package GAGE to test for changes in expression of the reported log 2 fold-changes reported by Limma in each term versus the background log 2 fold-changes of all genes found outside the respective term. The R/Bioconductor package heatmap3 was used to display heatmaps across groups of samples for each GO or MSigDb term with a Benjamini-Hochberg false-discovery rate adjusted p-value less than or equal to 0.05. Perturbed KEGG pathways where the observed log 2 fold-changes of genes within the term were significantly perturbed in a single-direction versus background or in any direction compared to other genes within a given term with p-values less than or equal to 0.05 were rendered as annotated KEGG graphs with the R/Bioconductor package Pathview. To find the most critical genes, the raw counts were variance stabilized with the R/Bioconductor package DESeq2 and then analyzed via weighted gene correlation network analysis with the R/Bioconductor package WGCNA. Briefly, all genes were correlated across each other by Pearson correlations and clustered by expression similarity into unsigned modules using a power threshold empirically determined from the data. An eigengene was then created for each de novo cluster and its expression profile was then correlated across all coefficients of the model matrix. Because these clusters of genes were created by expression profile rather than known functional similarity, the clustered modules were given the names of random colors where grey is the only module that has any pre-existing definition of containing genes that do not cluster well with others. These de-novo clustered genes were then tested for functional enrichment of known GO terms with hypergeometric tests available in the R/Bioconductor package clusterProfiler. Significant terms with Benjamini-Hochberg adjusted p-values less than 0.05 were then collapsed by similarity into clusterProfiler category network plots to display the most significant terms for each module of hub genes in order to interpolate the function of each significant module. The information for all clustered genes for each module was then combined with their respective statistical significance results from Limma to determine whether or not those features were also found to be significantly differentially expressed.

### n) Statistical Analysis

Shapiro-Wilk normality tests were conducted for the samples. The two-group independent t-test, one-way analysis of variance (ANOVA), and two-way ANOVA were used if the variables satisfied the normality assumption. Otherwise, the Mann-Whitney U test, Kruskal-Wallis one-way ANOVA test, and Friedman two-way ANOVA test were used. Bonferroni correction was used for post hoc multiple comparison in ANOVA. Statistical analyses were performed with Prism 6 (GraphPad Software).

### Example 2: Expression of SVEP1 in VSMCs in Human and Mouse Arteries

To characterize the role of SVEP1 in an atherosclerosis model, the following experiments were conducted to identify relevant tissues and cell types that express SVEP1 in both normal and atherosclerotic conditions in humans and mice.

Artery and left internal mammary artery (LIMA) tissues from human patients were co-stained with VSMCs using anti-smooth muscle alpha-actin (SMα-actin) antibody and expression of SVEP1 RNA was visualized using RNAscope ISH. SVEP1 was mainly expressed in the medial layer, and expression was mostly co-localized with VSMCs of human arteries including LIMAS and coronary arteries from the patients.

An in situ hybridization study was conducted using SVEP1 deleted mice (SVEP1^{+/-}) from a Knockout Mouse Project (KOMP). Because SVEP1^{-/-} mice died from edema at embryonic day 18.5, SVEP1^{+/-} mice were maintained and bred with Apoe^{-/-} mice to generate SVEP1^{+/-}Apoe^{-/-} mice. Because the SVEP1 targeting construct of the KOMP mice contained a lacZ reporter gene under the SVEP1 promotor, 3-galactosidase (β-gal) expression was assessed as a proxy for SVEP1 RNA expression. High expression of β-gal was observed in the medial layer of the aortic root, brachiocephalic artery (BCA), lesser curvature (LC), and thoracic aorta of 8-week-old SVEP1^{+/-}Apoe^{-/-} mice, and was also mainly co-localized with VSMCs. A similar analysis performed using similarly-aged Apoe^{-/-} (control) mice confirmed no β-gal expression in these regions.

SVEP1 RNA expression in the atherosclerosis-prone aortic root was assessed using in situ hybridization with RNAscope ISH co-stained with VSMCs using anti-SMα-actin antibody in the mice phenotypes as described above. SVEP1 expression was detected at a low level in both 8-week-old Apoe^{-/-} and SVEP1^{+/-}Apoe^{-/-} mice. After 8 weeks of high-fat diet feeding, SVEP1 was more induced and co-localized with VSMCs migrated into plaque lesions from Apoe^{-/-} mice as compared to SVEP1^{+/-}Apoe^{-/-} mice.

Without being limited to any particular theory, SVEP1 is known as a secreted ECM protein and is typically proteolytically processed into fragments both in vivo and in vitro. In a culture model, VSMCs were isolated from the eight-week-old-Apoe^{-/-} and SVEP1^{+/-} Apoe^{-/-} mice and incubated in the presence of 50 μg/mL oxLDL or vehicle control for 24 and 48 hrs. Secretion of SVEP1 was confirmed by Western blot with an anti-SVEP1 Ab. Both full-length (upper) and cleaved (lower) forms of SVEP1 protein were released into the conditioned medium. Aortic VSMCs isolated from Apoe^{-/-} mice showed more increase in both full-length and cleaved forms of SVEP1 protein secreted into conditioned media in response to pro-atherogenic oxidized low-density lipoprotein (oxLDL) compared to those of SVEP1^{+/-}Apoe^{-/-} mice. Taken together, SVEP1 appeared to be mainly expressed by VSMCs and was induced in plaque lesions during the development of atherosclerosis.

The results of these experiments demonstrated that SVEP1 was expressed in VSMCs in both human and mouse arteries and that SVEP1 was more induced during the development of atherosclerosis.

### Example 3: Effects of Whole-Body Knockout of SVEP1 in Mice.

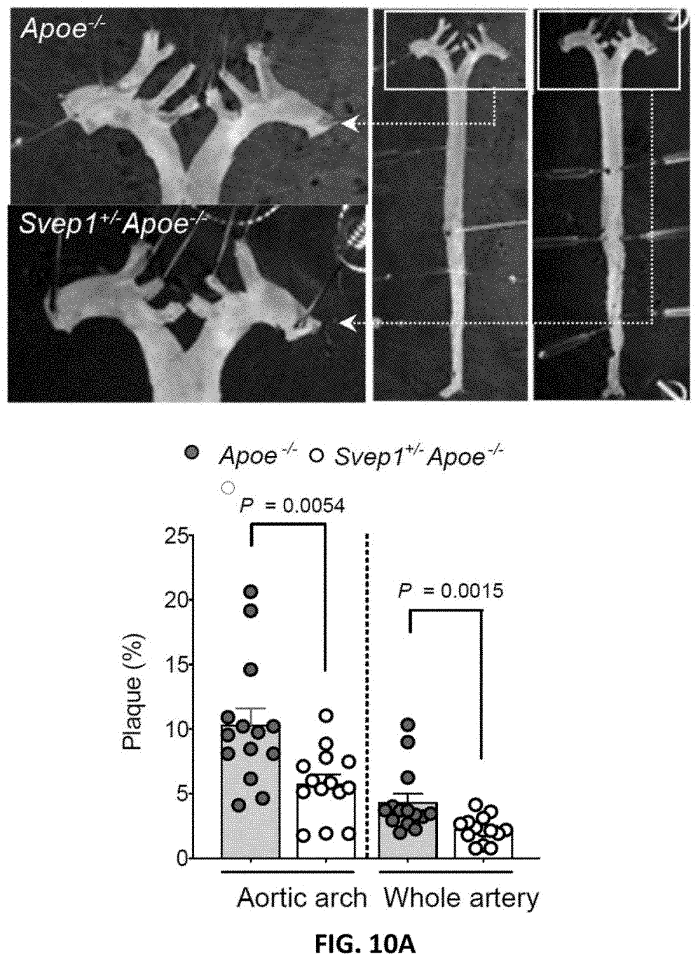

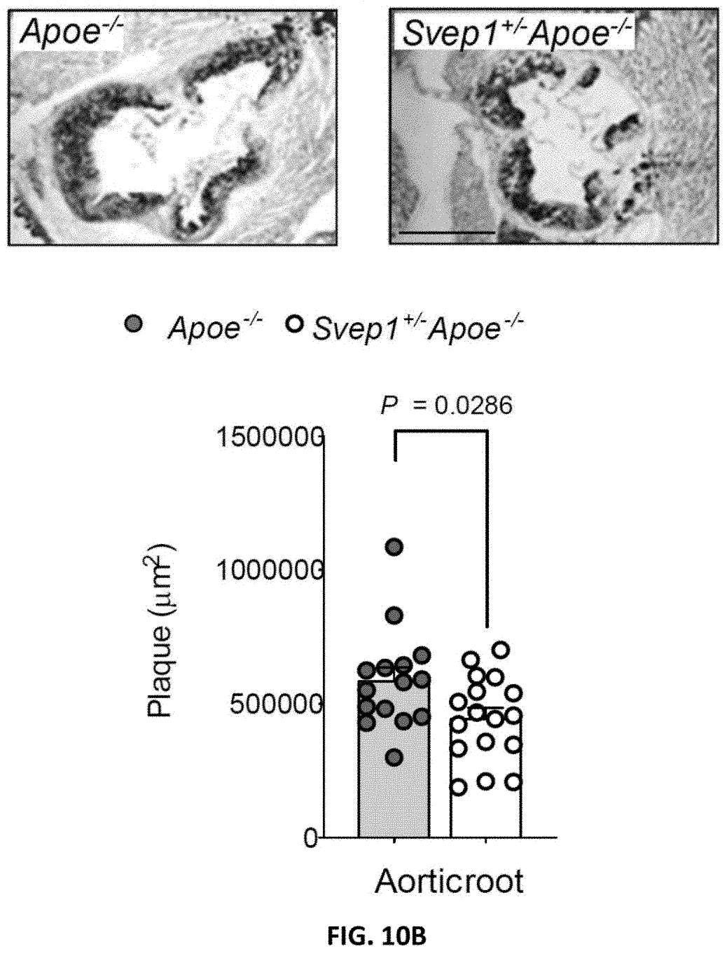

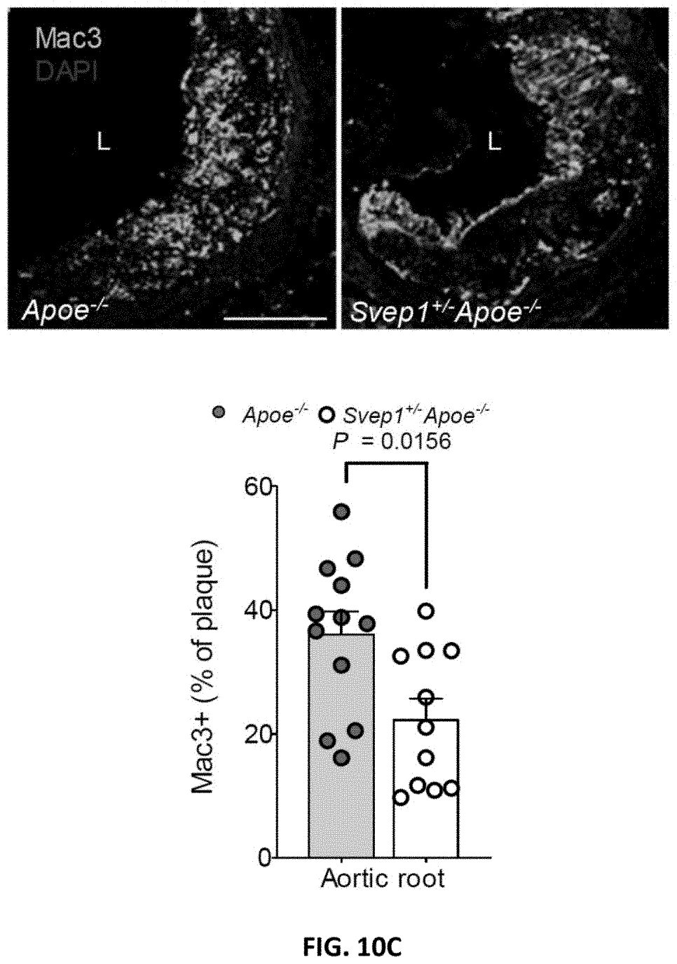

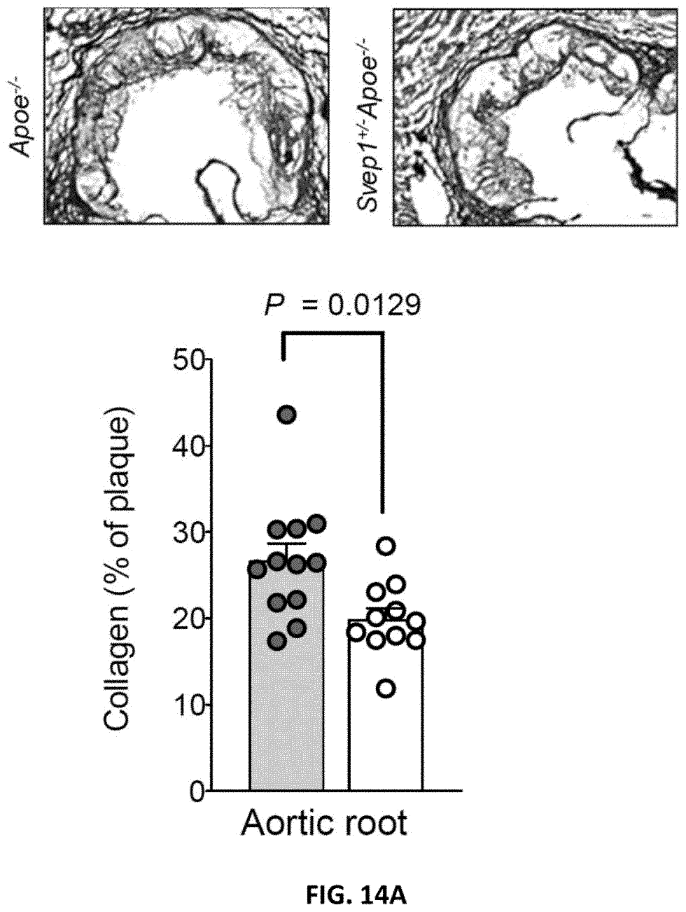

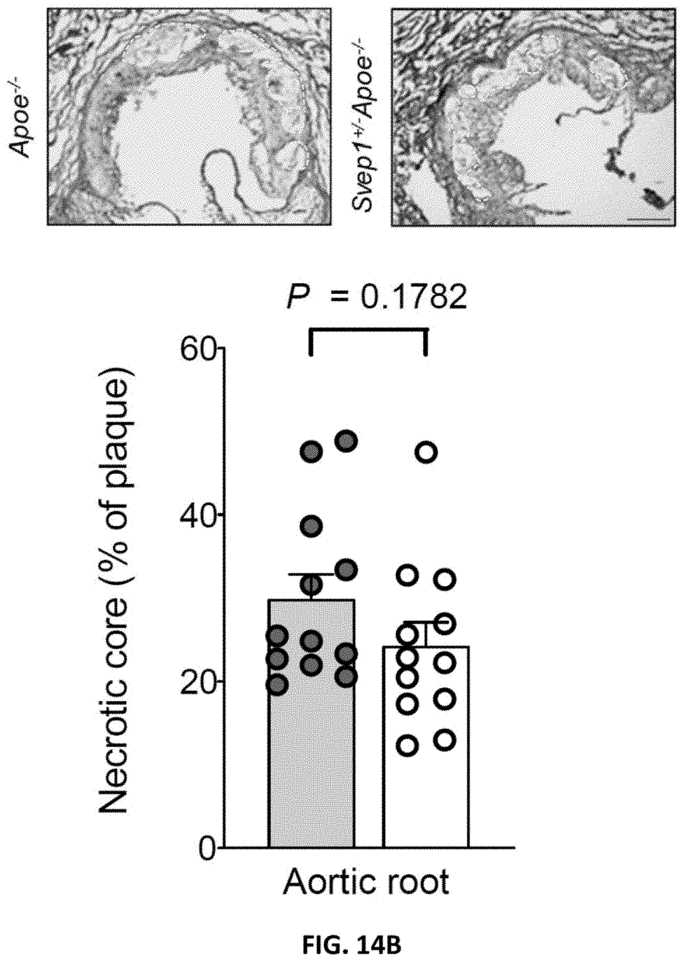

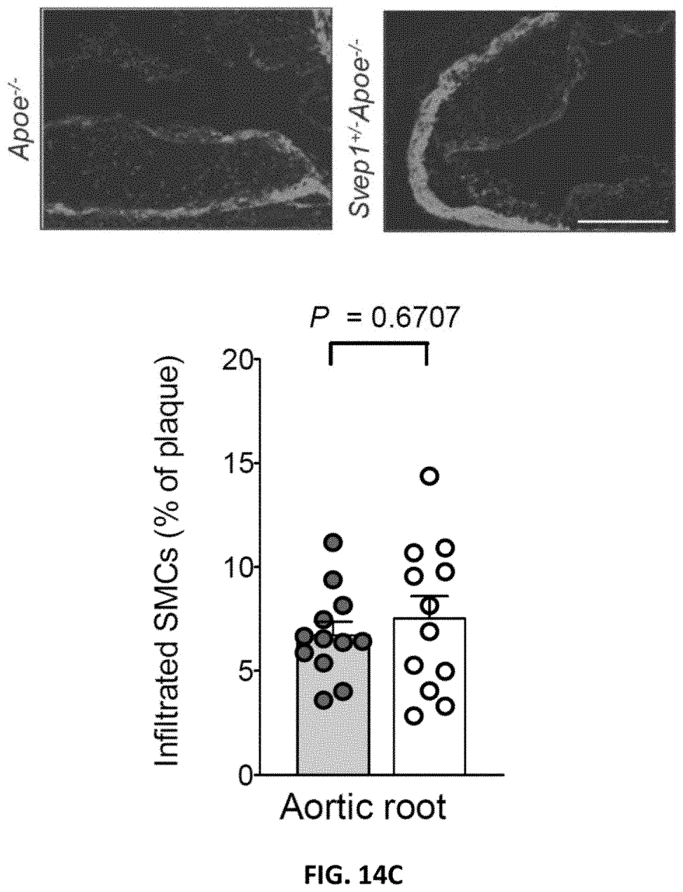

To study the role of SVEP1 in the development of atherosclerosis, the following experiments were conducted. Apoe^{-/-} and SVEP1^{+/-}Apoe^{-/-} mice similar to those described in Ex. 1 were fed with a high-fat diet for 8 weeks, and subjected to analysis for the formation of plaque lesions. Apoe−/− and SVEP1+/−Apoe−/− mice exhibited no differences in body weight, serum total cholesterol, triglyceride, or glucose. However, SVEP1+/−Apoe−/− mice showed significant decreases in plaque lesions in the aortic arch and whole artery in views of en face aortas (FIG. 10A) as well as in the aortic root (FIG. 10B). As illustrated in FIG. 10C, immunofluorescent staining of aortic root sections for Mac3 indicated that infiltration of macrophages into plaque lesions in the aortic root was significantly higher in Apoe−/− mice compared to SVEP1+/−Apoe−/− mice after 8 weeks of high-fat diet feeding. There was no difference in necrotic core size (see FIG. 14B) and amounts of infiltrated SMCs (see FIG. 14C) in plaques between the mice groups, whereas collagen maturation was slightly increased in Apoe−/− mice compared to SVEP1+/−Apoe−/− mice (see FIG. 14A).

Without being limited to any particular theory, SVEP1 is known as a potent ligand for Itg α9β1, and may be a factor involved in the formation of atherosclerosis as mediated by SVEP1 in the in vivo model described above. Itg α9β1 expression was assessed in the mice groups described above for three monocyte subsets (neutrophils, Ly6Chi monocytes, and Ly6Clow monocytes) according to a gating strategy. Both Ly6Chi and Ly6Clow monocytes showed high levels of Itg α9β1 expression, whereas neutrophils showed a low expression of Itg α9β1 expression in mouse blood.

Itg α9β1 expression was similarly assessed for four monocyte subsets in human blood: neutrophils, non-classical CD14-CD16+ monocytes, intermediate CD14+CD16+ monocytes, and classical CD14+CD16− monocytes. Itg α9β1 was highly expressed in neutrophils compared to non-classical CD14-CD16+, intermediate CD14+CD16+, and classical CD14+CD16− monocytes in human blood.

To assess Itg α9β1 expression in response to oxLDL treatment, the following experiment was conducted. Splenocytes isolated from Apoe−/− mice were incubated in vitro with 400 U collagenase D for 30 min at 37° C., followed by incubation with 25 μg/mL oxLDL or vehicle control (PBS) for 48 hrs. Macrophages, DCs, neutrophils, and Ly6Chigh monocytes were assessed according to the gating strategy. Both macrophages and Ly6chi monocytes showed constitutive Itg α9β1 expression. Itg α9β1 expression was similarly assessed in B cells and various T cell types (cytotoxic CD8+, regulatory CD8+, effector CD4+, and regulatory CD4+ T cells) according to the gating strategy. As illustrated in FIG. 10D, Itg α9β1 was not expressed in B cells or the T cell types including cytotoxic CD8+, regulatory CD8+, effector CD4+, and regulatory CD4+ T cells. None of the splenocytes described above expressed SVEP1, confirming that SVEP1 was mainly expressed in VSMCs, not in any myeloid lineage cells.

The expression of Itg α9β1 in myeloid cell types (neutrophils, dendritic cells (DC), macrophages, and Ly6C^{hi} monocytes) in the aortas of Apoe^{-/-} and SVEP1^{+/-} Apoe^{-/-} mice after 8 weeks of high-fat diet feeding was further analyzed, given the higher expression of Itg α9β1 in Ly6C^{hi} monocytes described above. Distinct subpopulations of aortic leukocytes were separated using their cell surface markers according to the gating strategy. Itg α9β1 was highly expressed in both macrophages and Ly6C^{hi} monocytes compared to neutrophils and DCs in these mice.

The results of these experiments demonstrated that whole-body loss of SVEP1 leads to a marked reduction in atherosclerotic plaque formation. Further, the results of these experiments determined that Itg α9β1 is highly expressed in monocytes and macrophages, and therefore may have a function in macrophages and monocytes during the development of atherosclerosis.

### Example 4: Effect of VSMC SVEP1 Modulation on Development of Atherosclerosis

To test the hypothesis that SVEP1 has a functional role in the regulation of VSMCs during the development of atherosclerosis, as well as to exclude the effects of SVEP1 expression in other cell types, the following experiments were conducted.

Apoe−/− mice were generated with VSMC-specific knockout of SVEP1 carrying a tamoxifen-inducible Cre-recombinase under the control of the VSMC-specific Myh11 promoter enhancer (SVEP1flox/floxMyh11-CreERT2Apoe−/−; referred to as SVEP1SMC fl/fl), along with wild-type SVEP1 (SVEP1+/+ Myh11-CreERT2Apoe−/−; referred to as SVEP1SMC +/+) mice as control.

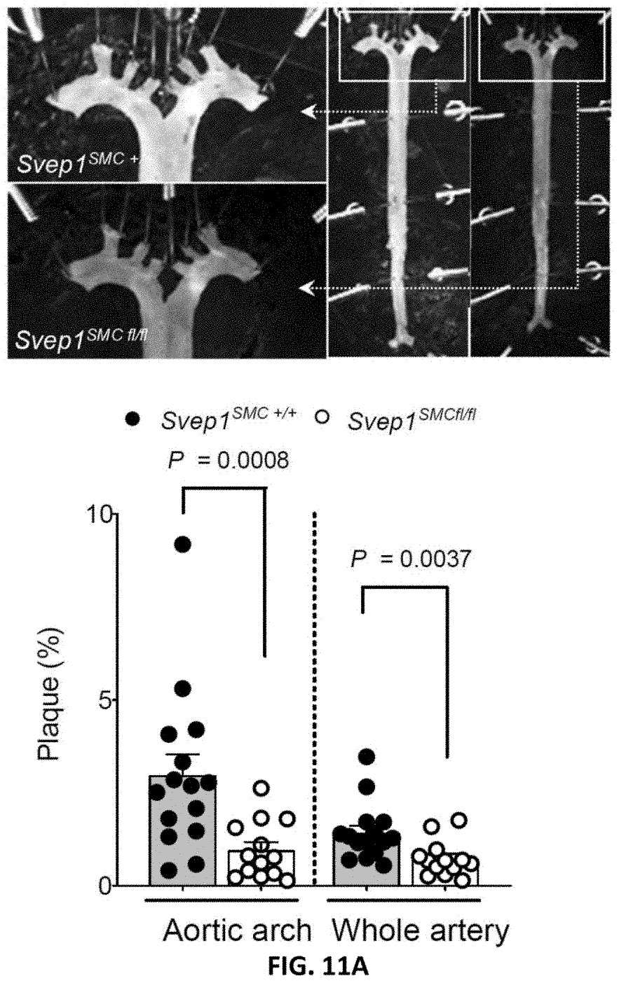

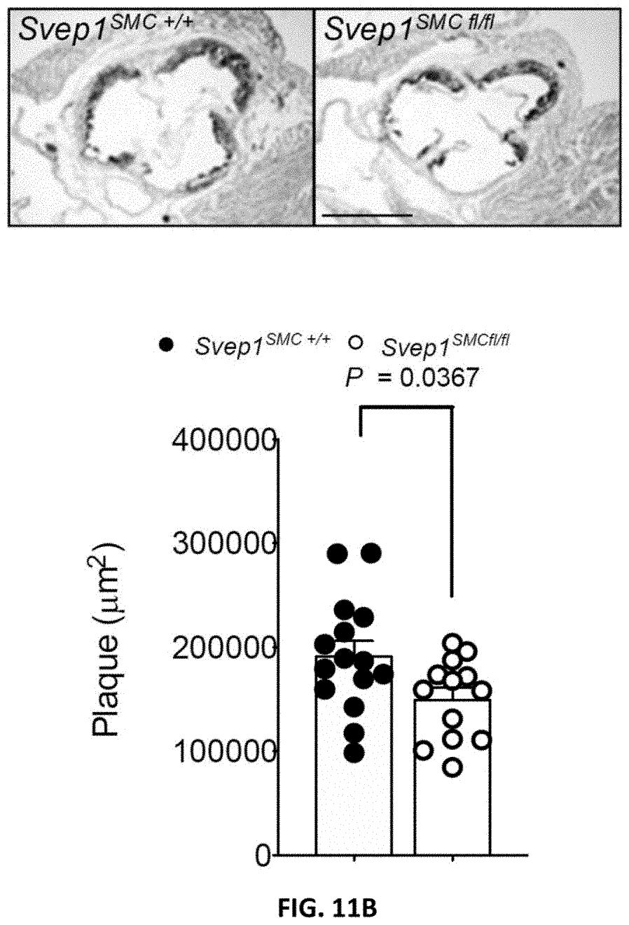

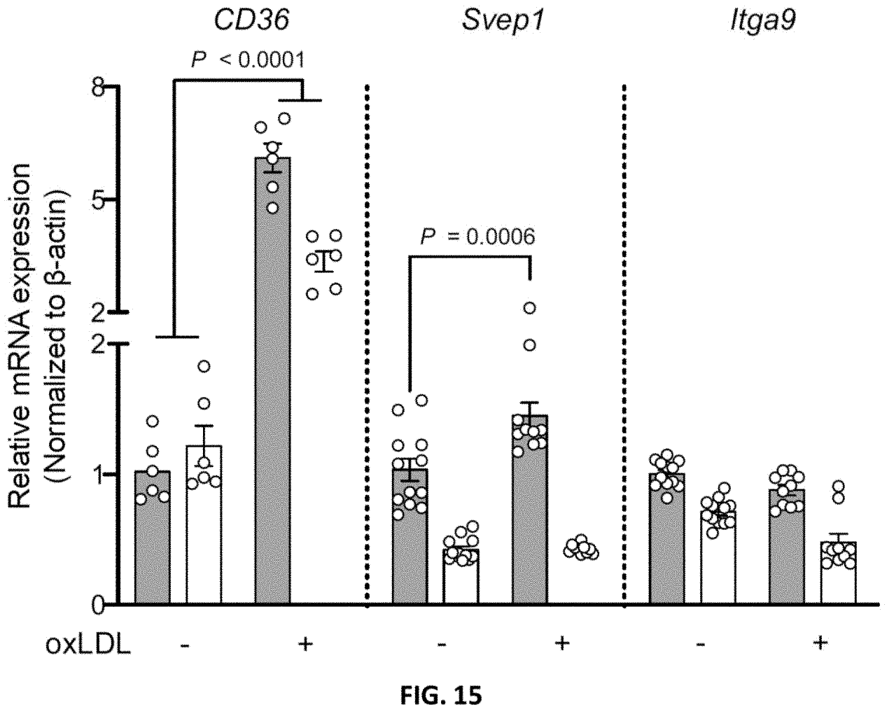

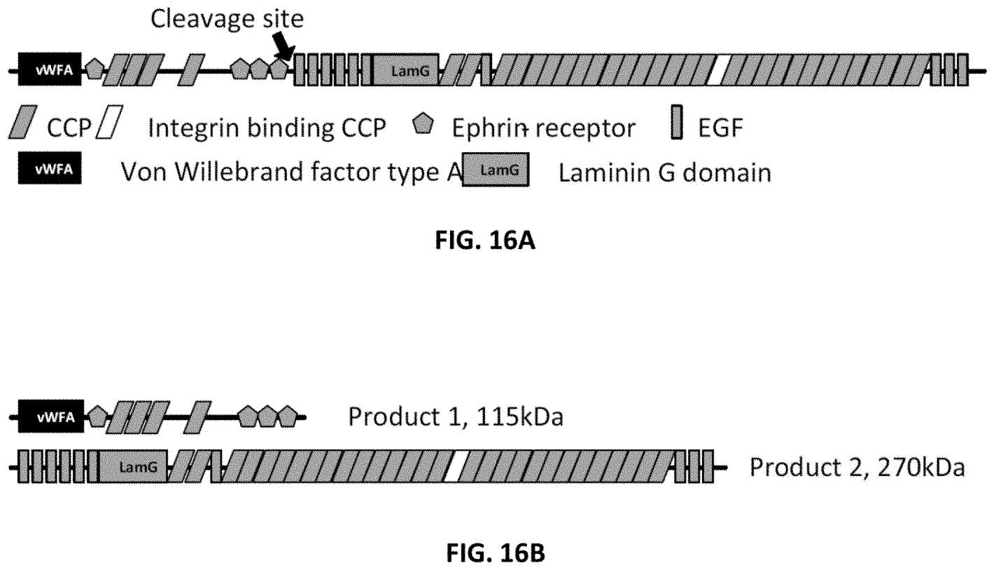

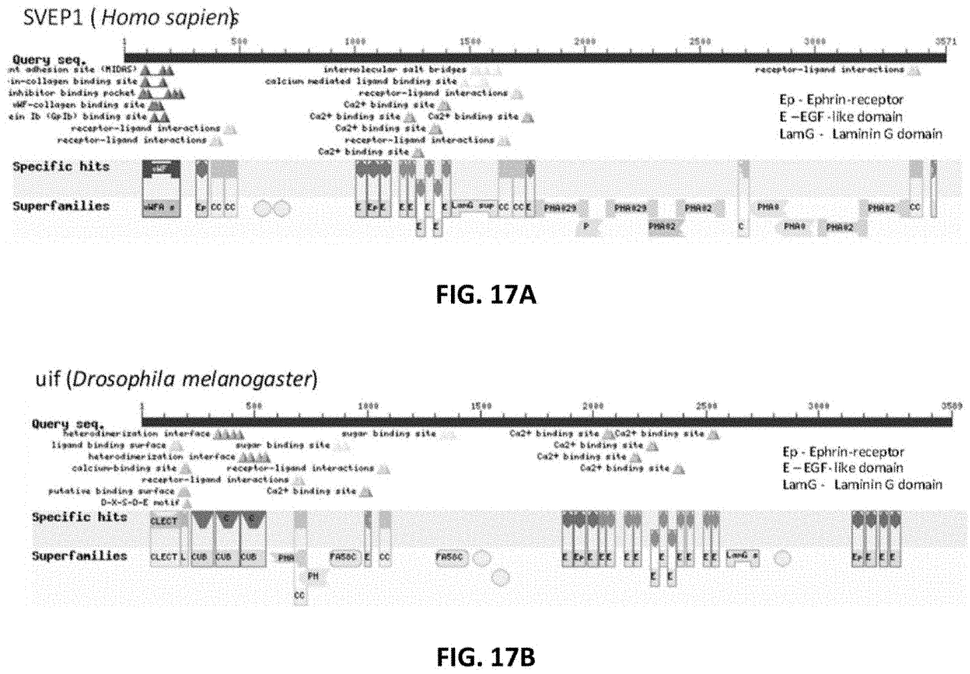

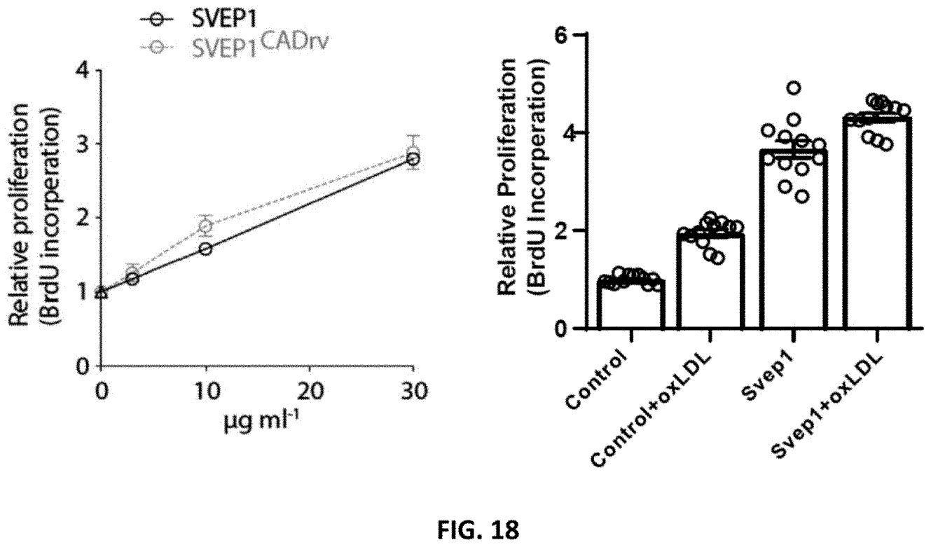

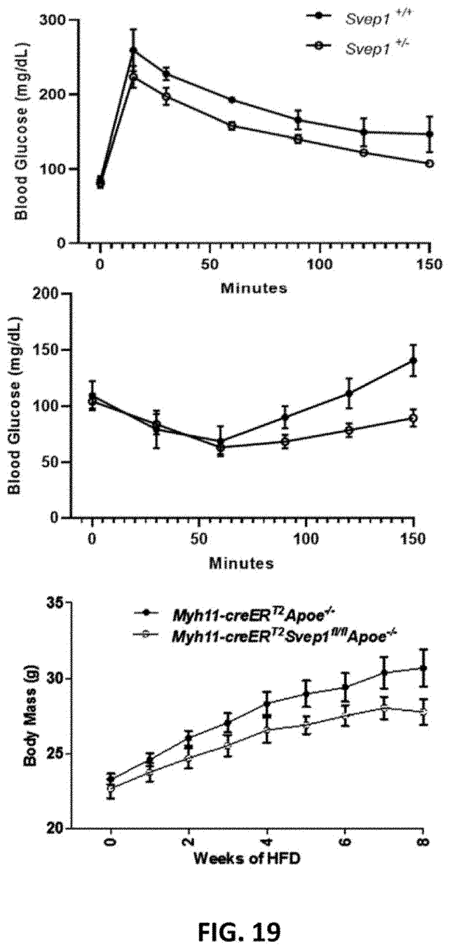

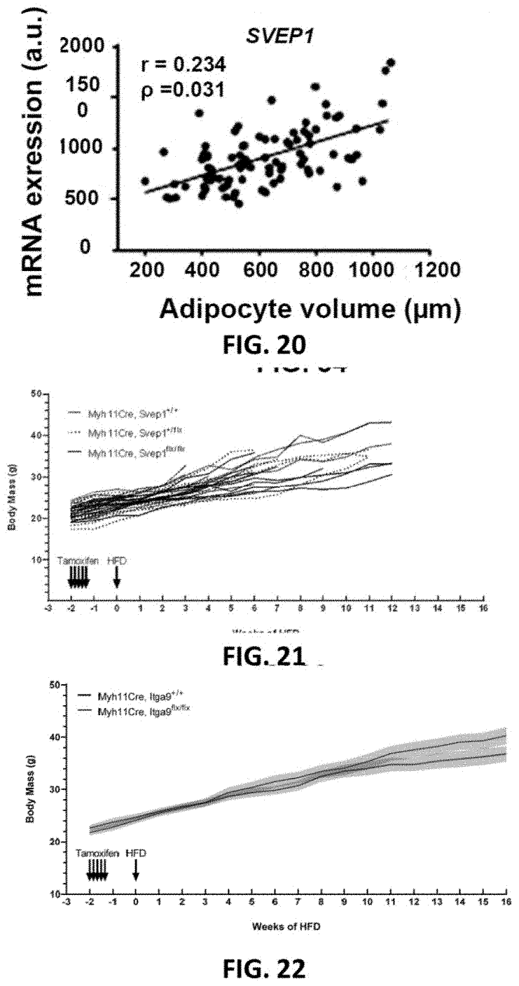

To assess the efficiency of SVEP1 recombination in VSMCs as described above, 10 intraperitoneal injections of tamoxifen were administered to the mice for 10 consecutive days starting at 6 weeks of age to activate Cre-recombinase for the depletion of SVEP1. Aortic VSMCs were isolated from SVEP1SMC fl/fl and SVEP1SMC +/+ mice at 8 weeks of age and incubated in the presence of 50 μg/mL oxLDL or vehicle control for 24 hrs. As illustrated in FIG. 15, VSMCs isolated from SVEP1SMC +/+ mice (green bars) showed 1.5 fold increase in SVEP1 mRNA expression after treatment of oxLDL, as compared to vehicle-treated control. However, this effect was nearly abrogated in VSMCs isolated from SVEP1SMC fl/fl mice (blue bars). VSMCs from both SVEP1SMC +/+ and SVEP1SMC +/+ mice expressed a high level of CD36 mRNA indicating that those cells were activated by oxLDL with high affinity. In mouse aortic tissues, in situ hybridization also showed that SVEP1 was fully depleted in VSMCs from both 8-week-old and high-fat diet fed SVEP1^{SMC+/+} mice. By contrast, SVEP1 was more induced and co-localized within VSMCs infiltrated into plaque and medial VSMCs in high-fat diet fed SVEP1^{SMC+/+} mice, as compared to 8-week-old same mice showing a basal level of SVEP1. After 8 weeks of high-fat diet feeding, SVEP1^{SMC fl/fl} mice showed a slight (non-significant) decrease in body weight compared to SVEP1^{SMC+/+} mice. There were no differences in total cholesterol, triglyceride, and glucose in plasma between these two groups. However, as illustrated in FIG. 11A, SVEP1^{SMC fl/fl} mice showed significant decreases in plaque lesion size in both the aortic arch and whole artery in views of en face aortas, as compared to SVEP1^{SMC}1^{+/+} mice. Although overall plaque lesion size in en face aortas of these SVEP1^{SMC+/+} and SVEP1^{SMC fl/fl} mice (FIG. 11A) was less than those in Apoe^{-/-} and SVEP1^{+/-}Apoe^{-/-} mice (FIG. 10A) with similar periods of high-fat diet feeding, these data suggested that loss of SVEP1 in VSMCs was more effective to prevent the formation of plaque lesion than whole body knockout of SVEP1. Plaque lesion size was also significantly decreased in the aortic root of SVEP1^{SMC fl/fl} mice, as compared to SVEP1^{SMC+/+} mice (FIG. 11B).

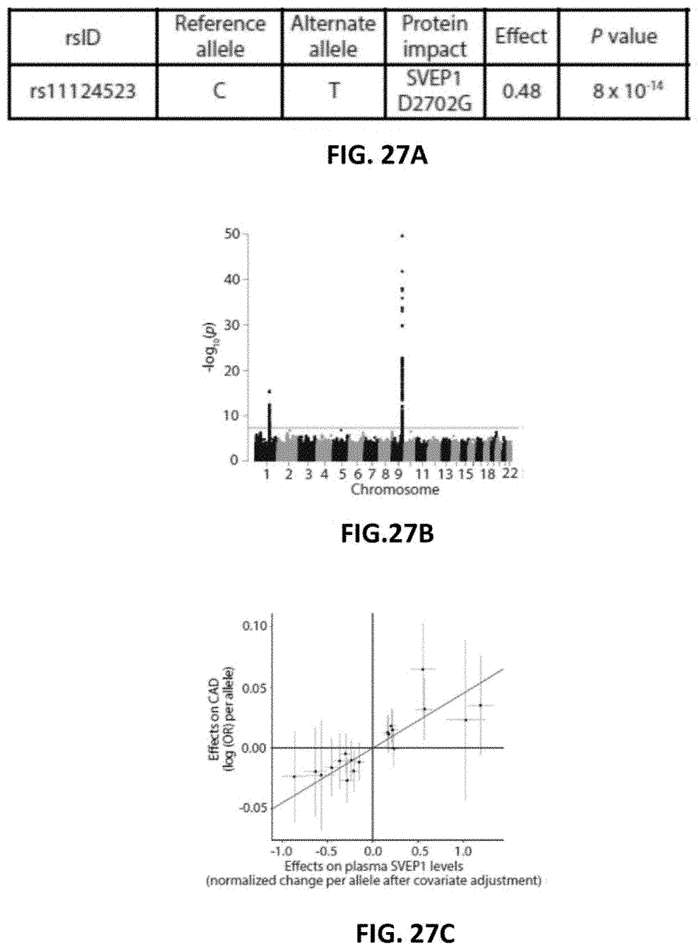

To investigate potential pathways involved in decreased atherosclerosis by loss of SVEP1 in VSMCs, RNA-seq analyses were performed on mRNA extracted from aortic arches of SVEP1^{SMC+/+} and SVEP1^{SMC fl/fl} mice fed a high-fat diet for 8 weeks. As shown in Table 1 below, loss of SVEP1 in VSMCs induced a significant downregulation of signaling and metabolism pathways during the development of atherosclerosis including cytokine-cytokine receptor interaction, chemokine signaling pathway, NF-kappa B signaling pathway, and TNF-signaling pathway. Notably, both ECM-receptor interaction and cell adhesion molecules (CAMs) were also downregulated in SVEP1^{SMC fl/fl} mice compared to SVEP1^{SMC+/+} mice as expected in terms of the mechanism of SVEP1 (FIG. 27A).

TABLE 1
Downregulated KEGG Pathways in SVEP1^{SMC fl/fl} Relative
to SVEP1^{SMC +/+} Mice After 8 Week Fat Diet

| | Number | | |
|---|---|---|---|
| Term_ID | of genes | P value | Fold difference |
| mmu04060 | 161 | 2.61811514886794E−09 | 6.2311140543057E−07 |
| Cytokine-cytokine | | | |
| receptor interaction | | | |
| mmu04640 | 68 | 1.81242500704374E−07 | 2.15678575838206E−05 |
| Hematopoietic | | | |
| cell lineage | | | |
| mmu04514 Cell | 105 | 4.35982947255434E−05 | 0.00345879804822644 |
| adhesion molecules | | | |
| (CAMs) | | | |
| mmu04062 Chemokine | 152 | 0.000127693398473063 | 0.00759775720914724 |
| signaling pathway | | | |
| mmu04662 B cell | 77 | 0.000163164582089827 | 0.00776663410747577 |
| receptor signaling | | | |
| pathway | | | |
| mmu04380 Osteoclast | 114 | 0.000407884267184576 | 0.0161794092649882 |
| differentiation | | | |
| mmu04080 Neuroactive | 108 | 0.000953733234422551 | 0.0324269299703667 |
| ligand-receptor | | | |
| interaction | | | |
| mmu04512 | 70 | 0.00124752678124566 | 0.0360062528402967 |
| ECM-receptor | | | |
| interaction | | | |
| mmu04145 Phagosome | 137 | 0.00136158098975912 | 0.0360062528402967 |
| mmu04650 Natural | 78 | 0.00292876682906862 | 0.0697046505318332 |
| killer cell mediated | | | |
| cytotoxicity | | | |
| mmu04610 | 55 | 0.00495014409295849 | 0.103386778073899 |
| Complement and | | | |
| coagulation cascades | | | |
| mmu04064 NF-kappa B | 86 | 0.00529882157332979 | 0.103386778073899 |
| signaling pathway | | | |
| mmu04151 PI3K-Akt | 280 | 0.00564717695361635 | 0.103386778073899 |
| signaling pathway | | | |
| mmu04672 Intestinal | 30 | 0.010944768713957 | 0.186061068137269 |
| immune network for | | | |
| IgA production | | | |
| mmu04630 Jak-STAT | 107 | 0.0150619670330227 | 0.238983210257293 |
| signaling pathway | | | |
| mmu04659 Th17 cell | 82 | 0.0196427666357311 | 0.292186153706499 |
| differentiation | | | |
| mmu04657 IL-17 | 67 | 0.0243280805855325 | 0.340593128197456 |
| signaling pathway | | | |
| mmu04668 TNF | 104 | 0.0285884727454224 | 0.366949570059212 |
| signaling pathway | | | |
| mmu04612 Antigen | 57 | 0.0310648794897438 | 0.366949570059212 |
| processing and | | | |
| presentation | | | |
| mmu04621 NOD-like | 141 | 0.0313293251992129 | 0.366949570059212 |
| receptor signaling | | | |
| pathway | | | |
| mmu04620 Toll-like | 79 | 0.0323779032405187 | 0.366949570059212 |
| receptor signaling | | | |
| pathway | | | |
| mmu04623 Cytosolic | 45 | 0.0347651767615785 | 0.376096003147986 |
| DNA-sensing pathway | | | |

Some pathways including oxidative phosphorylation, proteasome, protein export, and mRNA surveillance pathways were upregulated in SVEP1SMC fl/fl mice compared to control mice, although the associated p values were not as high as those associated with the downregulated pathways.

Quantitative PCR using cDNA from aortic arches was also performed to validate the RNA-seq analyses. Ccl2, Spp1, and Cxcl5 were significantly decreased in SVEP1SMC fl/fl mice, as compared to SVEP1SMC+/+ mice.

The results of these experiments indicated that SVEP1 was more induced in VSMCs under atherosclerotic conditions, and that loss of SVEP1 in VSMCs decreased atherosclerosis through the downregulation of signaling pathways mediated by SVEP1.

### Example 5: Effect of VSMC-Derived SVEP1 on Monocyte Recruitment and Macrophage Infiltration

To assess the effect of SVEP1 produced by VSMCs on monocyte recruitment and macrophage infiltration into plaques associated with atherosclerosis, the following experiments were conducted.

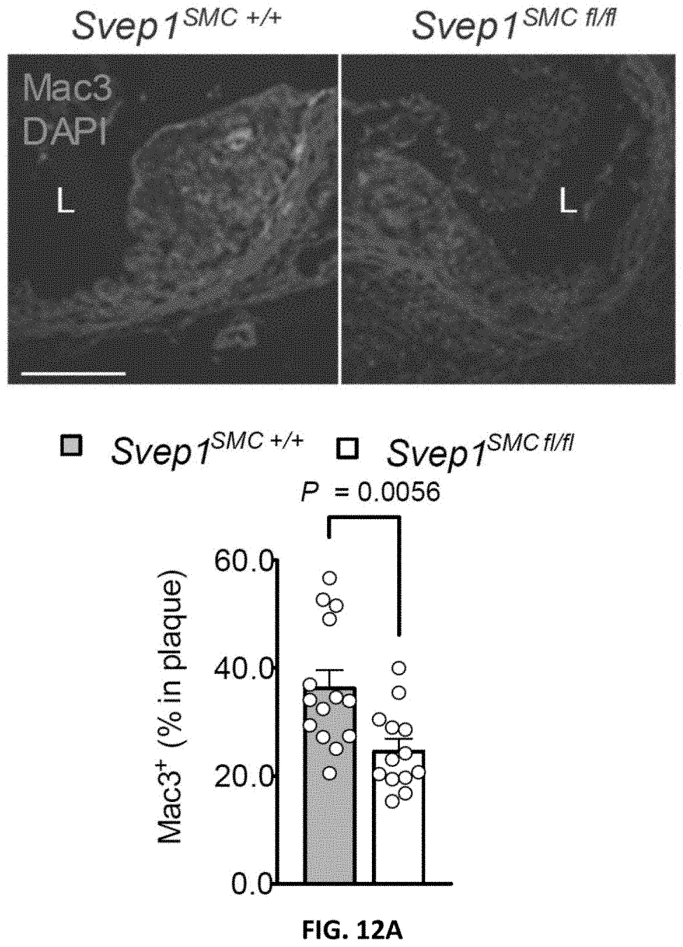

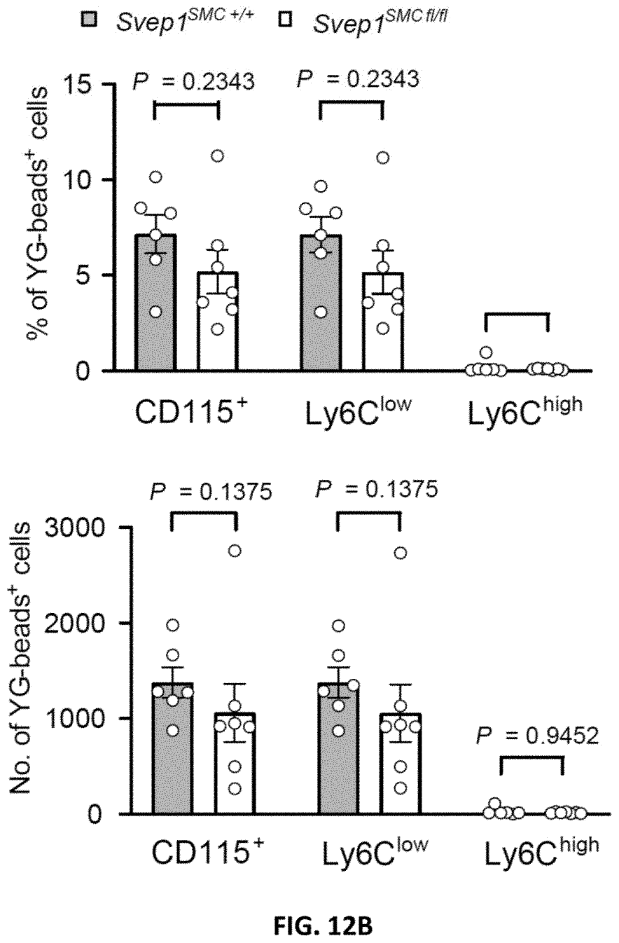

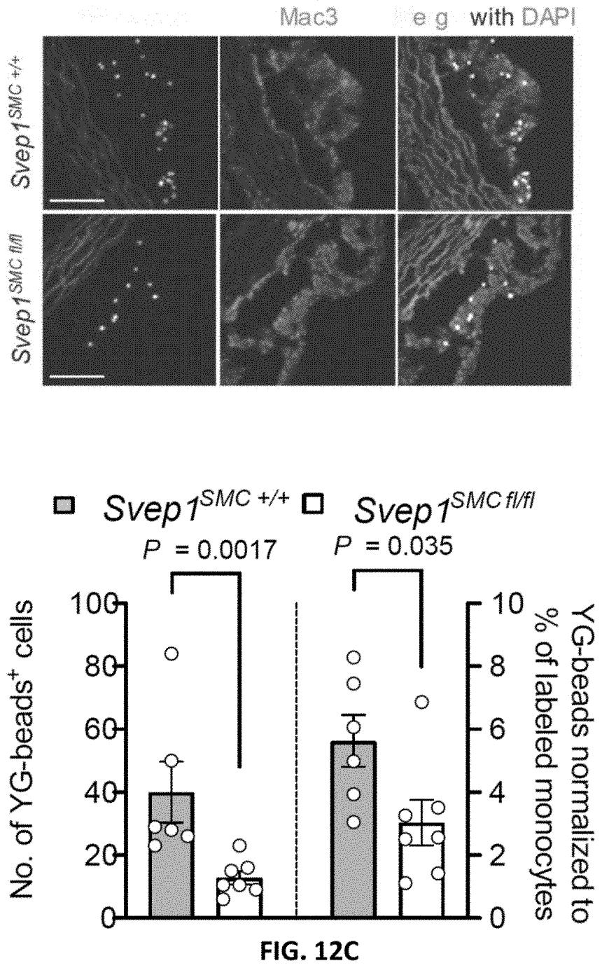

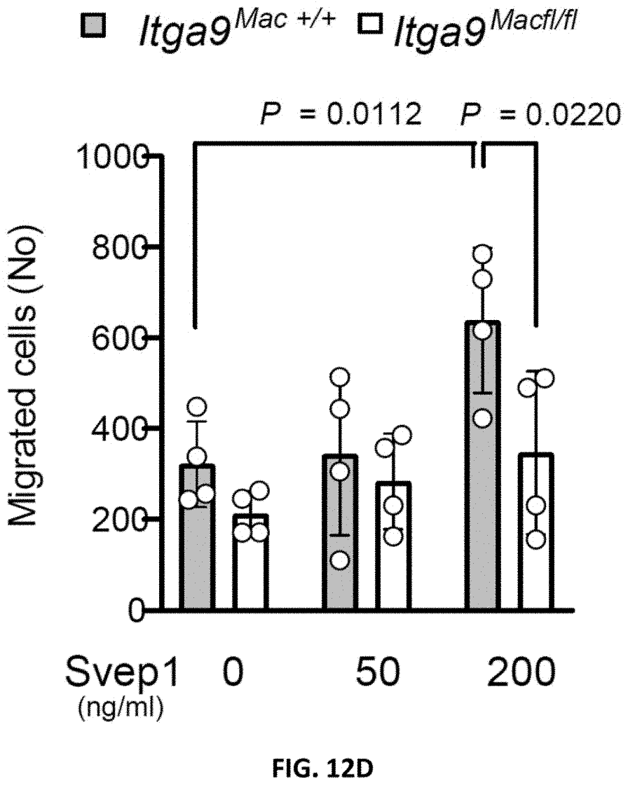

Aortic root sections were obtained from the SVEP1SMC fl/fl mice and SVEP1SMC+/+ after 8 weeks of high-fat diet feeding as described in Ex. 3 and subjected to immunofluorescent staining of aortic root sections for Mac3. As illustrated in FIG. 12A, SVEP1SMC fl/fl mice showed a significant reduction in infiltration of macrophages into plaque lesions as compared to SVEP1SMC+/+ mice. Analysis of mouse blood neutrophils and monocytes confirmed that Ly6C^{hi} monocytes expressed a high level of Itg α9β1 compared to Ly6Clow monocytes, whereas neutrophils showed a basal expression of Itg α9β1 in blood from both genotypes. This enhanced expression of Itg α9β1 on Ly6C^{hi} monocytes suggests that monocyte subsets may have a function involved in this event.

To determine whether the decreased amounts of macrophages in plaque were driven by decreased bone marrow-derived monocyte recruitment mediated by loss of SVEP1 in VSMCs, the following experiment was conducted. An in vivo monocyte recruitment assay was performed using yellow/green (YG) latex beads. After 8 weeks of high-fat diet feeding, we injected YG beads intravenously into both SVEP1SMC+/+ and SVEP1SMC fl/fl mice to label circulating Ly6Clow monocyte subsets, followed by flow cytometry performed 3 days after beads injection according to the gating strategy. As illustrated in FIG. 12B, the recruitment assay confirmed that YG beads labeled only Ly6Clow monocytes and not Ly6Chigh monocytes, suggesting that there was efficient bead labeling of circulating monocyte subsets in this assay. In addition, there was no difference in either percentages or absolute numbers of YG-beads detected within each of the monocyte subsets (total monocytes, CD11b+CD115+; Ly6Clow monocytes, CD11b+CD115+Ly6Clow; Ly6Chigh monocytes, CD11b+CD115+Ly6Chigh).

Aortic tissues were also obtained from the mice subjected to the YG bead assay for histology analysis. As illustrated in FIG. 12C, YG bead-labeled monocytes recruited into plaque lesions in the aortic root tissue immunostained for macrophages were quantified. Notably, SVEP1SMC fl/fl mice showed a significantly decreased recruitment of monocytes to plaque (with and without normalization to the percentages of labeled monocytes) compared to SVEP1SMC +/+ mice.

To better understand the interaction of SVEP1 with Itg α9β1 on monocytes and macrophages as related to the progression of atherosclerosis, the following experiment was conducted. Myeloid cell lineage-specific knockout of Itgα9mice were generated using LysM-Cre mice (Itgα9 flox/floxLysM-Cre; referred to as Itgα9 MAC fl/fl), along with wild-type Itgα9(Itgα9 +/+ LysM-Cre; referred to as Itgα9 MAC +/+) mice as control. An in vitro peritoneal macrophage migration assay using a trans-well apparatus was performed. The migratory responses of thioglycolate-elicited macrophages from Itg a SMAC +/+ and Itg a SMAC fl/fl were assessed in a chemotaxis chamber incubated with 0, 50, and 200 ng/mL of SVEP1 protein. As summarized in FIG. 12D, high concentrations of SVEP1 protein induced more migration of macrophages from both genotypes compared to vehicle control as expected. Moreover, macrophages isolated from Itgα9^{MAC+/+} mice showed more migration in response to SVEP1 protein compared to those from Itgα9^{MAC fl/fl} mice suggesting that SVEP1 increased migration of macrophages by interacting with Itg a9β1 on these cells.

### Example 6: Effect of SVEP1 on Proliferation and Phenotypic Transition of VSMCs

To characterize the functional properties and phenotypes of VSMCs in an atherosclerotic condition in vivo and in vitro, the following experiments were conducted.

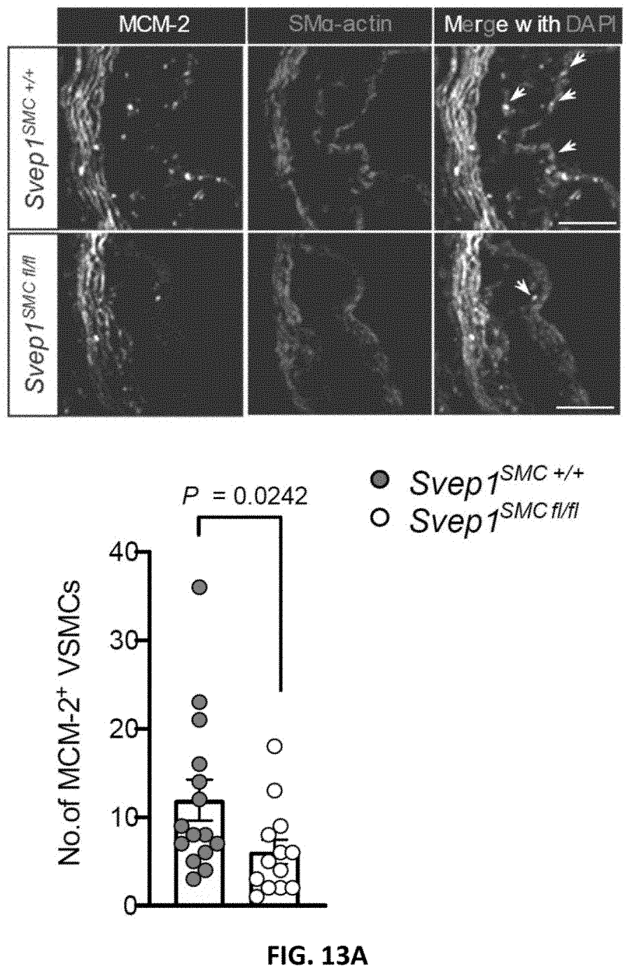

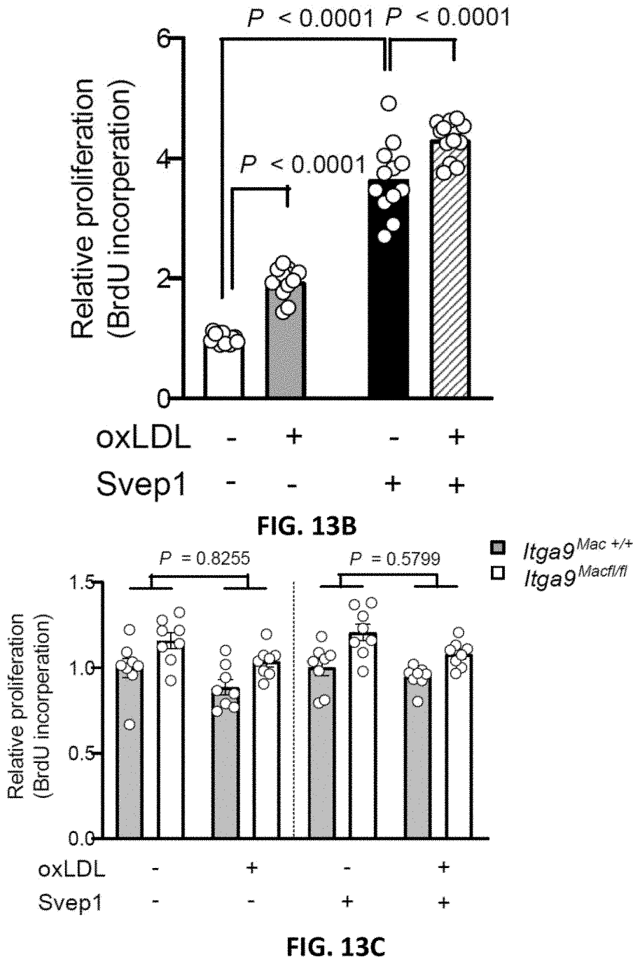

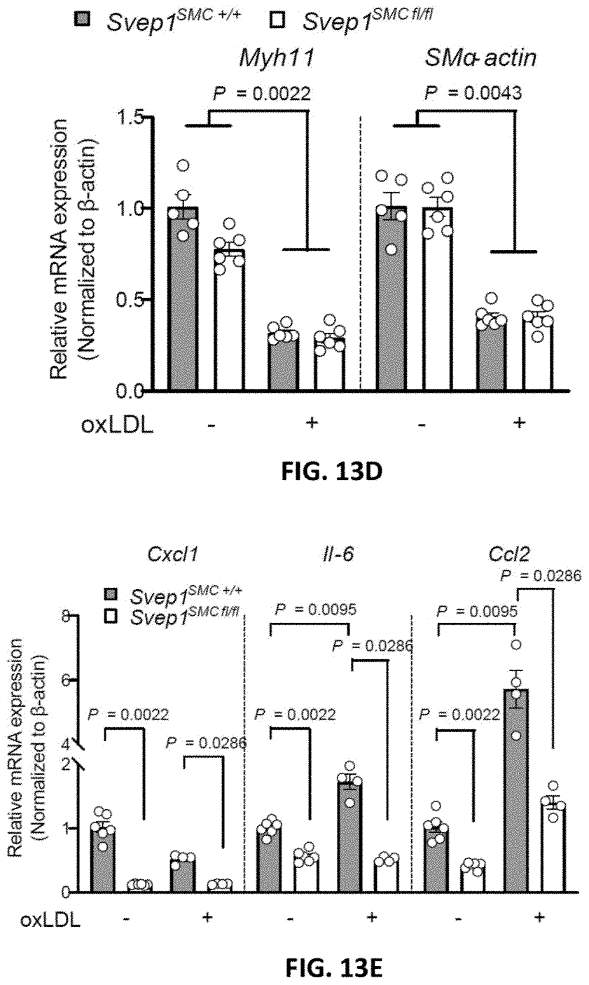

Loss of SVEP1 in VSMCs has functional properties on the formation of plaque related to the pathogenesis of atherosclerosis. Aortic root sections of the SVEP1SMC fl/fl mice and SVEP1SMC+/+ after 8 weeks of high-fat diet feeding as described in Ex. 3 were obtained for histological analysis. The proliferation of migrated VSMCs into the fibrous cap region was assessed by performing immunofluorescent staining using proliferation marker mini-chromosome maintenance protein-2 (MCM-2). Tissues were co-stained with VSMCs using anti-SMα-actin antibody. As summarized in FIG. 13A, the total number of MCM-2 positive VSMCs was significantly decreased in SVEP1SMC fl/fl mice, as compared to SVEP1SMC +/+ mice after feeding the high-fat diet for 8 weeks.

The effects of SVEP1 stimulation were assessed for various cell types using a BrdU assay, in which all cells were incubated with pre-coated 30 μg/mL SVEP1 protein or BSA (as vehicle control) for 8 hrs., and then treated with 50 μg/mL oxLDL in the culture media for 36 hrs. The culture plates were pre-coated with SVEP1 to mimic the distribution of matrix protein in a plaque environment. As summarized in FIG. 13B, stimulation of SVEP1SMC fl/fl VSMCs with oxLDL induced more proliferation compared to vehicle-treated control cells. Notably, the SVEP1 treatment in the presence of oxLDL was associated with a significant increase in the proliferation of SVEP1SMC fl/fl VSMCs, as compared to those of single oxLDL stimulated cells. Macrophages from the Itgα9 MAC +/+ and Itgα9 MAC fl/fl mice were similarly assessed using the BrdU assay. As illustrated in FIG. 13C, SVEP1 stimulation had no effect on the proliferation of cultured macrophages from Itgα9 MAC +/+ and Itgα9 MAC fl/fl mice in the presence of oxLDL, suggesting that SVEP1 might be a local factor implicated in regulating VSMC phenotype.

The phenotype transition of VSMCs under atherosclerotic conditions was assessed by quantifying the expression of macrophage markers on the VSMCs. SVEP1^{SMC+/+} and SVEP1^{SMC fl/fl} VSMCs were treated with 50 μg/mL oxLDL for 24 hrs., and markers for VSMCs (Myh11 and SMα-actin) were then quantified and normalized to β-actin mRNA expression. As summarized in FIG. 13D, after stimulation with oxLDL, both SVEP1^{SMC+/+} and SVEP1^{SMC+/+} VSMCs expressed lower levels of contraction markers including Myh11 and SMα-actin, as compared to vehicle-treated controls. As illustrated in FIG. 13E, stimulation with oxLDL induced dramatic increases in I1-6 and Ccl2 expression in SVEP1^{SMC+/+} VSMCs, but not Cxcl1 expression, relative to vehicle control. No effect of oxLDL treatment on the expression of these markers in SVEP1^{SMC fl/fl} VSMCs was observed. Notably, SVEP1^{SMC+/+} VSMCs expressed significantly higher levels of Cxcl1, I1-6, and Ccl2 as compared to SVEP1^{SMC fl/fl} VSMCs with and without treatment of oxLDL in this set of experiments. Taken together, these results indicated that SVEP1 increased the proliferation of VSMCs selectively, and modulated the phenotypic transition of these cells into macrophage-like cells under conditions representative of local atherosclerotic conditions.

### Example 7

Despite strong statistical evidence linking SVEP1 with CAD, its direct causality and potential disease-associated mechanisms were unclear. Here, if and how SVEP1 may influence the development of atherosclerosis is determined. Given the overlapping disease associations between SVEP1 and integrin α9β1, their shared biological functions, and the proximity of the variant to integrin α9β1's binding site, mechanistic studies were focused on cell types that play a prominent role in atherosclerosis and express either SVEP1, integrin α9β1, notch 3 or both of these genes.

### Materials and Methods

### Experimental Design

This study was designed to experimentally determine if SVEP1 causally contributes to the risk for atherosclerosis. Primary tissues harvested from humans and mice were used to determine if SVEP1 was produced in tissues and cell types of relevance to atherosclerosis. Mendelian Randomization was utilized to determine if SVEP1 is causally related to cardiovascular disease in humans. To study the mechanism by which SVEP1 may promote atherosclerosis, animal studies in HFD-fed Apoe−/− mice were performed. The number of animals used in each study group is specified in the figure legends. The in vitro experimental data included in this manuscript are representative of multiple experimental outcomes.

### Human Tissue Collection

Prior to coronary artery bypass grafting surgery (CABG) for the treatment of symptomatic coronary artery disease, five patients were consented for tissue and peripheral blood collection at the time of their planned CABG to be performed at Barnes Jewish Hospital. The surgical plan for all patients included using the left internal mammary artery (LIMA) as an arterial graft to the left anterior descending (LAD) coronary artery and at least one venous graft to a different coronary artery. During the CABG, the distal end of the LIMA was collected which was trimmed in order to accommodate the length needed to reach the LAD. The aortic wall punch biopsy was also collected and used to provide a proximal anastomotic site for the venous conduit. Tissues were immediately placed in phosphate-buffered saline (PBS) on ice and brought to the laboratory where they were frozen at −80° C. prior to in situ hybridization described below. During the CABG, 5-7 ml of peripheral blood was also collected in a tube containing the anticoagulant K3 EDTA (#6457, BD Biosciences) which was used for flow cytometry as described below.

### Mice

Svep1+/− mice were made by KOMP (knockout mouse project), and these mice were then crossed with mice expressing the flippase FLP recombinase under the control of the promoter of the human actin beta gene (hATCB) to generate Svep1flox/flox (Svep1Δ/Δ) mice. CRISPR/Cas9 genome editing technology was used in collaboration with the Washington University School of Medicine Genome Engineering and Transgenic Micro-Injection Cores to generate Svep1G/G mice on a C57BL/6 background harboring the SVEP1 mutation at the homologous murine position (p.D2699G). Svep1+/− and Svep1G/G mice were crossed with Apoe−/− mice (#002052, Jackson Laboratory) to get Svep1+/−Apoe−/− and Svep1G/+Apoe−/− mice, which were maintained as breeders to generate experimental and control mice. Svep1Δ/Δ mice were crossed with Myh11-CreERT2 (#019079, Jackson Laboratory) mice to generate Svep1Δ/+Myh11-CreERT2 mice. Svep1Δ/+Myh11-CreERT2 males were then crossed with Svep1Δ/+ females to generate experimental Svep1Δ/Δ Myh11-CreERT2 and control Svep1+/+Myh11-CreERT2 male littermate mice. Finally, Svep1Δ/Δ Myh11-CreERT2 males were crossed with Apoe−/− females. Svep1Δ/+Myh11-CreERT2Apoe−/− males and Svep1Δ/+Apoe−/− females were maintained as breeders to generate experimental Svep1Δ/Δ Myh11-CreERT2Apoe−/−(Svep1SMCΔ/Δ) and control Myh11-CreERT2Apoe−/− (Svep1SMC+/+) mice. To activate Cre-recombinase, mice were injected intraperitoneally with 1 mg of tamoxifen (#T5648, Sigma-Aldrich) in 0.1 mL peanut oil (#P2144, Sigma-Aldrich) for 10 consecutive days starting at 6 weeks of age. Tamoxifen treatment was performed with all experimental and control mice in an identical manner. Itgα9 flox/flox (Itgα9 Δ/Δ) mice were gifts from Drs. Dean Sheppard and Livingston Van De Water (Albany Medical College, New York), and LysM-Cre mice were provided by Dr. Babak Razani (Washington University School of Medicine, Saint Louis). Itgα9 fl/fl mice were crossed with LysM-Cre mice and maintained Itgα9fl/+LysM-Cre mics3e as breeders to generate Itgα9 fl/flLysM-Cre (Itgα9MACΔ/Δ) and control Itgα9+/+LysM-Cre (Itgα9MAC+/+) mice. All mice were housed in a pathogen-free environment at an animal facility and maintained on a 12 hr light/12 hr dark cycle with a room temperature of 22±1° C.

### Diet and Assessment of Atherosclerosis

All experimental mice were fed a diet containing 21% fat by weight (42% kcal from fat) and 0.2% cholesterol (#TD.88137, Envigo Teklad) for 8 and 16 weeks starting at 8 weeks of age. After HFD feeding, blood was collected from the retro-orbital plexus after 12 hr of fasting. Mice were euthanized by carbon dioxide inhalation. Plasma samples were prepared from the collected blood by centrifugation at 13,000 rpm for 10 min at 4° C. Total cholesterol (#STA-384), triglycerides (#STA-397), and glucose (#STA-681) in mouse plasma were determined using the appropriate kit (all purchased from Cell Biolabs, Inc). Hearts and whole aortas (from the aortic arch to the iliac artery) were harvested after perfusion with PBS. For en face analysis, isolated aortas were cleaned by removing perivascular fat tissues, opened longitudinally, and pinned onto black wax plates. After fixation with 4% paraformaldehyde overnight at 4° C., aortas were washed with PBS for 1 hr and stained with 0.5% Oil Red O in propylene glycol (#O1516, Sigma-Aldrich) for 3 hr at room temperature. After staining, aortas were de-stained with 85% propylene glycol in distilled water for 5 min to reduce background staining and washed with distilled water for 15 min. For analysis of plaque in the aortic root, hearts were fixed overnight with 4% paraformaldehyde at 4° C., washed with PBS for 1 hr, and embedded into OCT compound (#4583, Sakura® Finetek). 5-μm-thick cryosections were stained overnight with 0.5% Oil Red O in propylene glycol, de-stained with 85% propylene glycol in distilled water for 5 min, and washed with distilled water for 15 min. Measurement of plaque was performed using 6-8 sections per artery to get the average value of size. The atherosclerotic plaque area was digitized and calculated using AxioVison (Carl Zeiss).

### Antibodies and Reagents

To produce murine SVEP1 protein, total RNA was purified from the lung tissue of 8 week-old-mice by RNeasy® kit (Life Technology). SuperScript IV First-Strand Synthesis System (Life Technology) with oligo d(T)20 primer used to obtain full-length reverse transcripts. Following double-stranded DNA synthesis by PCR using PrimeSTAR GXL DNA Polymerase (Takara Bio) using forward 5′-ATGTGGTCGCGCCTGGCCTTTTGTTG and reverse 5′-AAGCCCGGCTCTCCTTTTCCTGGAACAATCAT primers, the resulting amplicon was subcloned into pCMV6 plasmid (Origene Technology) in frame with Myc and Flag tag by In-Fusion HD EcoDry Cloning Plus System (Takara Bio). In order to insert a poly histidine tag for protein expression and purification, the Flag tag was replaced by a 10-histidine tag. Oligonucleotides 5′ -CACCACCACCACCACCACCACCACCACCACTGAACGGCCGG and 5′ -CCGTTCAGTGGTGGTGGTGGTGGTGGTGGTGGTGGTG coding for poly-histidine, stop codon and Fsel compatible sticky end were synthetized, annealed and ligated to the vector, which was digested by EcoRV and Fsel downstream of the myc tag and the stop codon respectively. The sequence of the full Svep1-myc-His10 construct was verified by Sanger sequencing. The protein was expressed in FreeStyle 293F cells (Invitrogen) grown in FreeStyle expression media. Transient transfection of the 3 μg ml-l of vector DNA plus 9 μg ml-l Polyethylenimine (PEI) (25 kDa linear PEI, Polysciences, Inc.) was used to express the protein in 2.5×106 cells ml-l. After overnight incubation, the same volume of fresh media plus 1 mM (final concentration) of valproic acid was added. The transfected cells were incubated in flasks in an orbital shaker at 100 rpm at 37° C. with an 8% CO2 incubator for 48 hr, the conditioned media was collected by centrifugation at 300 rpm for 15 min. Cells were resuspended in fresh expression media and incubated for 48 hr. The conditioned media was collected, and this procedure was repeated one more time. Cell viability at the end of the experiment was >70%. The protein was purified in an NGC chromatographic system (BioRad Lab) in two steps: i) conditioned media was added to 1/10 of its volume of 10× PBS, and the pH was checked and fixed to 7.1. The protein was pulled down by a disposable column loaded with 5 ml Nuvia IMAC resin (BioRad Lab) at 1 ml min-1, washed with 50 ml PBS and the protein eluted in PBS plus 250 mM Imidazole, and ii) the eluted protein solution was concentrated 10 times in a Vivaspin concentrator 30 kDa cut off and loaded in a Superose 6 increase 10/300 (GE Life Sciences) with PBS as a carrier buffer. The fractions were evaluated by western blot probed with Myc tag antibody, and the purity of the protein was evaluated in PAGE-SDS 4-15% stained with Coomassie brilliant blue. More than 95% of the protein in the gel corresponded to a single band. Further analysis by Mass Spectroscopy confirmed that more than 95% of the peptides detected corresponded to SVEP1.

For immunofluorescent staining, anti-β-galactosidase (#ab9361, abcam, 1:1000), anti-Mac3 (#550292, clone M3/84, BD Biosciences, 1:100), anti-SMα-actin-cy3 (#C6198, clone 1A4, Sigma-Aldrich, 1:1000), anti-MCM-2 (#4007, Cell Signaling, 1:100) were used, and then visualized with anti-chicken-Alexa488 (#A11039), anti-rat-Alexa488 (#A21470), anti-rat-Alexa594 (#A21471, all purchased from Invitrogen, 1:400), and ProLongTM Gold antifade reagent with DAPI (#P36935, Invitrogen) were used. In case of detection of MCM-2 staining, samples were visualized with anti-rabbit-HRP (#7074S, Cell Signaling, 1:1000) followed by TSA® Plus Cyanine 5 (#NEL745E001KT, PerkinElmer). For the immunohistochemistry study, hematoxylin solution (#HHS80), eosin solution (#HT110180), Masson's trichrome staining kit (#HT15-1KT, all purchased from Sigma-Aldrich), and Permount solution (#SP15-500, Fisher Chemicals) were used. For flow cytometry, following anti-mouse antibodies were used; anti-CD16/32 FcR blocker (#14-0161, eBioscience), PerCP-labeled anti-CD45 (#103129, clone 30-F11), BV510-labeled anti-CD1lb (#101263, clone M1/70), BV421-labeled anti-CD64 (#139309, clone X54-5/7.1), PE/cy7-labeled anti-CD11 c (#117317, clone N418), APC/cy7-labeled anti-MHCII (#107627, clone M5/114.15.2), FITC-labeled anti-F4/80 (#123108, clone BM8), BV605-labeled anti-CD19 (#115540, clone 6D5), APC-labeled anti-CD115 (#135510, clone AFS98), Alexa700-labeled anti-Ly6C (#128023, clone HK1.4), PE/Cy7-labeled anti-Ly6G (#127618, clone 1A8, all purchased from Biolegend), PE/cy5.5-labeled anti-CD4 (#35-0042-82, clone RM4-5, eBioscience), Alexa700-labeled anti-CD8a (#56-0081-80, clone 53-6.7, eBioscience), and PE-labeled anti-Itgα9 f31 (#FAB3827P, R&D systems). Following anti-human antibodies were used. FcR blocker (#564219, BD biosciences), PerCP/cy5.5-labeled anti-CD45 (#368504, clone 2D1), Alexa700-labeled anti-CD3 (#300323, clone HIT3a), anti-CD19 (#115527, clone 6D5), anti-CD56 (#392417, clone QA17A16), FITC-labeled anti-CD15 (#301904, clone HI98), BV421-labeled CD66b (#305111, clone G10F5), APC/cy7-labeled anti-CD14 (#325619, clone HCD14), BV605-labeled anti-CD16 (#360727, clone B73.1), and PE-labeled anti-ITGα9β1 (#351606, clone Y9A2, all purchased from Biolegend). RBC lysis buffer (#423101, Biolegend), FoxP3 transcription factor staining buffer set (#00-5523, eBioscience), Leuko spin medium (#60-00091, Pluriselect) were used in flow cytometry experiments. Antibodies for western blotting include: Src (#2109), P-Src (#6943) FAK (#3285), P-FAK (#8556), Paxillin (#2542), P-Paxillin (#2541), Erk (#4695), P-Erk (#4370), p38 (#8690), P-p38 (#9211, all purchased from Cell Signaling Technologies).

### Immunohistochemistry and Immunofluorescent Staining

For all immunohistochemistry and immunofluorescent study, 4% paraformaldehyde-fixed frozen sections with 5-p.m-thickness were used. For immunofluorescent staining, slides were air-dried for 1 hr at room temperature and hydrated with PBS for 10 min. After permeabilization with 0.5% tritonX-100 for 10 min, sections were blocked with PBS containing 5% chicken serum (#S-3000, Vector Laboratories) with 0.5% tritonX-100 for 1 hr at room temperature. And then slides were incubated with the indicated antibodies. For hematoxylin and eosin (H&E) staining, air-dried slides were hydrated in PBS for 10 min, placed in hematoxylin solution for 10 min, and then rinsed in running tap water. After de-staining in 1% acetic acid for 5 min, slides were rinsed in tap water and placed in 90% ethanol for 5 min. Slides were stained with eosin solution for 8 min, gradually dehydrated in ethanol solution (from 80% to 100%), and then incubated with xylene for 10 min followed by mounting with Permount solution. For Masson's trichrome staining, air-dried slides were hydrated in distilled water for 10 min, placed in Mordant in Bousin's solution for 1 hr at 56° C., and rinsed in running tap water for 5 min. After staining in hematoxylin solution for 10 min, slides were washed in running tap water for 10 min, rinsed in distilled water, placed in Biebrich scarlet-acid fuchsin solution for 15 min, and stained in aniline blue solution for 10 min. After rinsing in distilled water, slides were differentiated in 1% acetic acid for 3 min, gradually dehydrated in ethanol solution (from 80% to 100%), and incubated with xylene for 10 min followed by mounting with Permount solution.

### RNAscope In Situ Hybridization (ISH)

To detect RNA transcripts for SVEP1 and ITGA9 in both human and mouse vascular tissues, a commercially available kit (#323100, RNAscope® Multiplex Fluorescent Reagent Kit v2, Advanced Cell Diagnostics) was used according to the manufacturer's instructions. Briefly, 4% paraformaldehyde-fixed mouse aortic root and human aortic wall, and LIMA frozen sections with 5-μm-thickness were air-dried for 1 hr at room temperature and treated with hydrogen peroxide for 10 min to block endogenous peroxidase activity. After antigen retrieval by boiling in target antigen retrieval solution for 5 min at 95-100° C., slides were treated with protease III for 30 min at 40° C. Target probes (#406441, mouse Svep 1; #540721, mouse Itgα9 ; #811671, human SVEP1; #811681, human ITGA9) were hybridized for 2 hr at 40° C., followed by a series of signal amplification and washing steps. Hybridization signals were detected by TSA® Plus Cyanine 5, and co-stained with indicated antibodies. Slides were counterstained with DAPI by using ProLong™ Gold antifade reagent.

### Flow Cytometry

For labeling mouse blood cells, blood was collected from the retro-orbital plexus, and red blood cells were removed using RBC lysis buffer (#00-4300-54, eBioscience). For labeling human blood cells, Leuko spin medium (Pluriselect) was used to isolate leukocytes from peripheral blood and buffy coat. In an experiment using mouse spleen, spleen cells were recovered from mice by cutting the spleen into small fragments, and incubated with 400 U collagenase D (#11-088-858, Roche applied science) for 30 min at 37° C. For labeling aortic single-cell suspensions, isolated aortas were perfused with DPBS, and opened longitudinally. The whole artery was cut into 2-5 mm pieces and incubated in a Hanks' Balanced Salt Solution (HBSS) solution with calcium and magnesium containing 90 U ml-l DNase I (#DN25), 675 U ml-l collagenase I (#C0130), 187.5 U ml-l collagenase XI (#C7657), and 90 U ml-l hyaluronidase (#H1115000, all purchased from Sigma-Aldrich) for 70 min at 37° C. with gentle shaking. Non-specific binding to Fc receptors was blocked, and cells were incubated with the indicated antibodies for 30 min at 4° C. For intracellular staining, cells were fixed/permeabilized with the FoxP3 transcription factor staining buffer set. Flow cytometric analyses were performed using LSRFortessaTM instrument (BD Biosciences) and FlowJo software (Tree Star Inc).

Bead Labeling of Ly6Clow Monocytes Recruited into Atherosclerotic Plaque

After 8 weeks of HFD feeding, 200 μL of 1 μm Fluoresbrite yellow-green (YG) microspheres beads (#17154-10, Polysciences, Inc) diluted 1:4 in sterile DPBS were administered retro-orbitally. The labeling efficiency of blood monocytes was verified by flow cytometry 3 days after the YG bead injection. Recruitment of YG-beads positive monocytes into plaque in the aortic root was analyzed 1 day after checking the labeling efficiency of YG beads. 5-μm-thick frozen sections of aortic root were stained with anti-Mac3, followed by anti-rat-Alexa594 antibody. And slides were mounted with ProLong™ Gold Antifade Mountant with DAPI. The number of YG-beads colocalized with Mac3 positive area was counted, or normalized with the percentages of labeled Ly6Clow monocytes.

### Primary Cell Isolation and Culture

Mouse aortic VSMCs were isolated from 8-week-old Apoe−/− and Svep1+/−Apoe−/−, or same age of Svep1SMC+/+ and Svep1SMCΔ/Δ mice after tamoxifen injection for 10 consecutive days starting at 6 weeks of age. Briefly, thoracic aortas were harvested (3 mice per group were used), perivascular fat was removed, and then aortas were digested in 1 mg ml-l collagenase II (#LS004174), 0.744 units ml-l elastase (#LS002279), 1 mg ml-l soybean trypsin inhibitor (#LS003570, all purchased from Worthington Biochemical Corporation), and 1% penicillin/streptomycin in HBSS for 10 min at 37° C. with gentle shaking. After a pre-digestion with enzyme mixture, the adventitial layer was removed under the dissection microscope, and the intimal layer was removed by scrapping with forceps. The aortas were cut into small pieces and completely digested in an enzyme mixture at 37° C. for 1 hr with gentle shaking. VSMCs were grown in 20% fetal bovine serum (FBS, Hyclone) containing Dulbecco modified Eagle medium/F12 (DMEM/F12) media (Gibco) with 100 U ml-l penicillin/streptomycin at 37° C., 5% CO2 incubator. After 2 passages, VSMCs were changed to 10% serum. To stimulate VSMCs, 50 μg ml-l human medium oxidized low-density lipoprotein (oxLDL, #770202-7, Kalen biomedical) was used. Human primary CASMCs were obtained in their third passage from Invitrogen. Cells were grown in Medium 231 with Smooth Muscle Growth Supplement (Gibco) and passaged according to the manufacturer's instructions. Medium lacking growth supplement was used in cell assays.

### Quantitative Real-Time PCR

Gene expression was quantified by quantitative real-time PCR. RNA was isolated using RNeasy® Mini Kit (#74106, Qiagen) according to the manufacturer's protocol, and QIAshredder homogenizer (#79656, Qiagen) to increase the yield of quantification of RNA. cDNA was synthesized with High Capacity cDNA Reverse Transcript Kit (#4368814). Real-time PCR was performed using both Taqman® (#4444557) and SYBRTM Green (#A25742, all purchased from Applied Biosystems) assays. Ct values were normalized to β-actin (for Taqman®) and gapdh (for SYBRTM Green) and showed as expression relative to control. A list of probes and primers used is shown in Table S3.

TABLE S3
List of oligonucleotides used for qPCR studies.
Taqman®

| Gene | Oligo Catalog No. | Company |
|---|---|---|
| Svep1 | Mm00465702_m1 | ThermoFisher Scientific |
| SVEP1 | Hs00225829_m1 | ThermoFisher Scientific |
| CD36 | Mm00432403_m1 | ThermoFisher Scientific |
| Itgα9 | Mm00519317_m1 | ThermoFisher Scientific |
| β-actin | Mm02619580_m1 | ThermoFisher Scientific |
| β- | Hs99999903_m1 | ThermoFisher Scientific |
| ACTIN | | |

SYBR™ Green

| Gene | Forward primer | Reverse primer |
|---|---|---|
| Ccl2 | CAAGAAGGAATGGGTCCAGA | GCTGAAGACCTTAGGGCAGA |
| Spp1 | TCACCATTCGGATGAGTCTG | ACTTGTGGCTCTGATGTTCC |
| Cxcl5 | ATCACCTCCAAATTAGCGATCA | TTCTGTTGCTGTTCACGCT |
| SMα- | GCATCCACGAAACCACCTA | CACGAGTAACAAATCAAAGC |
| actin | | |
| Myh11 | CATGGACCCGCTAAATGACA | CAATGCGGTCCACATCCTTC |
| Cxcl1 | GCTTGAAGGTGTTGCCCTCAG | AAGCCTCGCGACCATTCTTG |
| Il-6 | AGTTGCCTTCTTGGGACTGA | TCCACGATTTCCCAGAGAAC |
| Hey1 | GAAGCGCCGACGAGACC | CAGGGCGTGCGCGTCAAAATAACC |
| | GAATCAA | |
| Hey2 | CGACGTGGGGAGCGAGAACAAT | GGCAAGAGCATGGGCATCAAAGTA |
| Heyl | AGACCGCATCAACAGCA | CAAGTGATCCACGGTCAT |
| Hes1 | GACGGCCAATTTGCTTTC | GACACTGCGTTAGGACCC |
| Gapdh | TCACCACCATGGAGAAGGC | GCTAAGCAGTTGGTGGTGCA |

In Vitro Migration Assay using Peritoneal Macrophage

Itgα9 MAC+/+ and Itgα9MACΔ/Δ mice were injected intraperitoneally with 1 ml 4% thioglycolate. After 5 days, the peritoneal cells were collected by lavage and placed in RPMI media containing 10% FBS for 60 min at 37° C. Non-adherent cells were removed after washing with PBS 3 times, and adherent cells (more than 90% were peritoneal macrophages confirmed by flow cytometry) were placed in Trans-well inserts with a 5-μm porous membrane in a modified Boyden chamber. RPMI media containing 10% FBS with 50 or 200 ng ml-l SVEP1 protein was placed in the lower chamber. After allowing cell migration of 16 hr, inserts were removed from the upper side of the chamber, and nuclei of migrated cells to the lower side of the membrane were stained with DAPI. The number of migrated cells was determined by In Cell Analyzer 2000 (GE healthcare).

### Proliferation and Adhesion Assays

Wells of a 96-well plate were pre-coated with 30 μg ml-l recombinant SVEP1 protein or bovine serum albumin (BSA, as an inert protein control). Wells were subsequently blocked with 10 mg ml-l BSA and washed twice with DPBS. Plates were ultraviolet (UV) sterilized before adding cells. For proliferation assays, primary VSMCs were collected and suspended in DMEM/F12 media containing 10% FBS. 2,000 cells were added to each well and incubated for 8 hr to assure complete cell adhesion. Media was then replaced with DMEM/F12 media containing 0.2% BSA and incubated for 12 hr to reduce basal proliferation rates. Cells were then incubated in BrdU dissolved in DMEM/F12 media containing 0.2% BSA for 30 hr. Predesigned Silencer Select siRNA constructs targeting Itgα9 and negative control siRNA were obtained from ThermoFisher. Primary VSMCs were transfected using RNAiMAX transfection reagents according to the manufacturer's protocol. Efficient Itgα9 knockdown was confirmed by qPCR. Cells were trypsinized 24 hr after transfection and used for the proliferation assay. DAPT or DMSO (carrier) was added to cells throughout the indicated experiment at a concentration of 25 μM. A similar protocol was adapted to test the proliferation of human CASMCs, with a 24 hr incubation in BrdU-containing media. For using peritoneal macrophages, 4% thioglycolate-elicited peritoneal macrophages from Itgα9 MAC+/+ and Itgα9 MACΔ/Δ mice were suspended in BrdU-containing RPMI 1640 media that also contained 10% FBS. 25,000 cells were added to each well since peritoneal macrophages have lower proliferation rates than VSMCs in culture. 50 μg ml-l oxLDL was added to the indicated cells at the beginning of this incubation. An ELISA for incorporated BrdU was then performed using kit instructions (#6813, Cell Signaling Technologies) after incubation for 36 hr. Adhesion assays were performed in precoated 96-well plates blocked with 100 mg ml-l BSA. Blocking conditions were empirically derived to minimize non-specific cell adhesion. After 5 min (for THP-1 cells) or 15 min (for VSMCs) incubation, non-adhered cells were removed by gently centrifuging the plates upside down. VSMCs were counted manually and THP-1 cells were counted by automated microscopy after staining cells with DAPI.

### Western Blotting Assay

Cells were resuspended in serum-free media (SFM) and incubated with gentle agitation to prevent cell attachment and reduce basal signaling. Cells were washed with SFM and then seeded on BSA-blocked plates coated with either BSA, VCAM-1, SVEP1, or SVEP1CADrv. Concentrations of VCAM-1 and SVEP1 were derived empirically to prevent signal saturation. BSA concentrations always matched the SVEP1 concentration. It is only appropriate, therefore, to compare between BSA, SVEP1, and SVEP1CADry groups. Cells were briefly centrifuged to the bottom of the wells and incubated for 8 min (for VSMCs) or 15 min (for THP-1 cells) before lysis with cell lysis buffer (#9803, Cell Signaling Technologies) containing a cocktail of protease and phosphatase inhibitors. Western blots were performed by standard techniques, as briefly follows. Protein content was determined using a bicinchoninic acid assay with BSA standards (#23225, Pierce™ BCA Protein Assay Kit). Cell lysates were then reduced with DTT in lithium dodecyl sulfate sample buffer (#NP0007, Invitrogen). Equal protein amounts were added to polyacrylamide gels (#4561086, BioRad) and electrophoresed prior to transferring to a nitrocellulose or polyvinylidene fluoride membrane (#1620260, BioRad). Membranes were blocked in 5% BSA/Tris-Buffered Saline with tween 20 for 30 min. The indicated primary antibodies were incubated with the pre-blocked membranes for overnight at 4° C. Membranes were washed with Tris-Buffered Saline with tween 20, probed with fluorescent secondary antibodies, and imaged. β-actin or β-tubulin served as a loading control.

### Bulk RNA Sequencing and Analysis

Primary VSMCs were plated on wells precoated with 30 μg ml-l recombinant SVEP1, SVEP1CADry protein or BSA (as an inert protein control). Wells were subsequently blocked with 10 mg ml-l BSA and washed twice with DPBS. Plates were UV sterilized before adding cells. Primary VSMCs were collected, resuspended in DMEM/F12 media containing 10% FBS, plated on precoated, blocked wells, and incubated for 8 hr to ensure complete cell adhesion. Media was replaced with fresh DMEM/F12 containing 1% FBS and incubated for 12 hr before collection. RNA was collected using RNeasy® Mini Kit (#74106, Qiagen). Atherosclerotic aortic arches (including the aortic root, arch, and the proximal regions of its branching vessels) from mice were used as the source of RNA for the later RNAseq experiment. These tissues were isolated and separated from the perivascular adipose prior to storing in RNAlater (#AM7021, Thermofisher) prior to total RNA extraction using nucleoZOL (Macherey-Nagel). cDNA for validation was synthesized with High Capacity cDNA Reverse Transcript Kit (#4368814, Applied Biosystems), following standard protocols.

Samples were prepared according to the library kit manufacturer's protocol, indexed, pooled, and sequenced on an Illumina HiSeq. Basecalls and demultiplexing were performed with Illumina's bcl2fastq software and a custom python demultiplexing program with a maximum of one mismatch in the indexing read. RNA-seq reads were then aligned to the Ensembl release 76 primary assembly with STAR version 2.5.1a. Gene counts were derived from the number of uniquely aligned unambiguous reads by Subread:featureCount version 1.4.6-p5 2). Isoform expression of known Ensembl transcripts was estimated with Salmon version 0.8.2. Sequencing performance was assessed for the total number of aligned reads, the total number of uniquely aligned reads, and the features detected. The ribosomal fraction, known junction saturation, and read distribution over known gene models were quantified with RSeQC version 2.6.2. All gene counts were then imported into the R/Bioconductor package EdgeR and TMM normalization size factors were calculated to adjust for samples for differences in library size. Ribosomal genes and genes not expressed in the smallest group size minus one sample greater than one count-per-million were excluded from further analysis. The TMM size factors and the matrix of counts were then imported into the R/Bioconductor package Limma. Weighted likelihoods based on the observed mean-variance relationship of every gene and sample were then calculated for all samples with the voomWithQualityWeights. The performance of all genes was assessed with plots of the residual standard deviation of every gene to their average log-count with a robustly fitted trend line of the residuals. Differential expression analysis was then performed to analyze for differences between conditions and the results were filtered for only those genes with Benjamini-Hochberg false-discovery rate adjusted P-values less than or equal to 0.05. One sample in the aortic arch experiment was independently identified as an outlier by standard quality control methods. This group was excluded from downstream analyses.

For each contrast extracted with Limma, global perturbations in known Gene Ontology (GO) terms, KEGG pathways, and InterPro domains were detected using the Database for Annotation, Visualization and Integrated Discovery (DAVID) on significantly dysregulated transcripts or using the R/Bioconductor package GAGE to test for changes in expression of the reported log 2 fold-changes reported by Limma in each term versus the background log 2 fold-changes of all genes found outside the respective term. The R/Bioconductor package heatmap3 was used to display heatmaps across groups of samples for each GO or MSigDb term with a Benjamini-Hochberg false-discovery rate adjusted p-value less than or equal to 0.05. Perturbed KEGG pathways where the observed log 2 fold-changes of genes within the term were significantly perturbed in any direction compared to other genes within a given term with P-values less than or equal to 0.05 were rendered as annotated KEGG graphs with the R/Bioconductor package Pathview.

### Notch Signaling Assays

Cells were collected, resuspended in SFM, and incubated for 1 hr with gentle agitation before seeding on tissue culture wells that were precoated and blocked, as described in previous sections. Cells were collected for analysis after 4 hr of growth on the indicated substrate. Svep1SMC+/+ and Svep1SMCΔ/Δ VSMCs were collected for analysis after 72 hr of incubation in SFM to obtain basal Notch signaling. Notch target gene primers used for the qPCR are listed in Table S3.

### Analysis of Cytokine and Chemokine Biomarkers

MILLIPLEX MAP Mouse Cytokine/Chemokine Magnetic Bead Panel-Immunology Multiplex Assay (#MCYTOMAG-70K), MILLIPLEX MAP Mouse Angiogenesis/Growth Factor Magnetic Bead Panel-Cancer Multiplex Assay (#MAGPMAG-24K), and MILLIPLEX MAP Mouse Cardiovascular Disease (CVD) Magnetic Bead Panel 1-Cardiovascular Disease Multiplex Assay (#MCVD1MAG-77K-02, all from Millipore Sigma) were used to analyze cytokines and chemokines from mouse plasma. All kits were used according to manufacturer-recommended protocols. Briefly, the Luminex FLEXMAP 3D® (Luminex Corporation, Austin, Tex.) instrument was used to sort the magnetic polystyrene beads and measure the phycoerythrin (PE) tagged detection antibody signal. Fifty beads from each analyte were measured. The median fluorescent intensity (MFI) was compared against the standard curve to calculate the pg ml-l or ng ml-l using Milliplex Analyst 5.1 software (VigeneTech.com) and a 5-parameter logistic curve fit algorithm.

### BMDM Isolation and Culture

6- to 8-week-old Itgα9 MAC+/+ and Itgα9 MACΔ/Δ mice were euthanized by carbon dioxide inhalation and soaked in 75% ethanol. Then, femurs and tibias were harvested and bone-marrow cells were obtained by flushing bones and differentiated for 7 days in DMEM media supplemented with 50 mg ml-l recombinant macrophage-colony stimulating factor (M-CSF, R&D systems), 20% heat-inactivated FBS, and antibiotics.

### Mendelian Randomization

To estimate the causal effect of SVEP1 plasma protein levels on the risk of CAD, hypertension, and type 2 diabetes (T2D), Mendelian Randomization using summary statistics from publicly available datasets was performed. Genome-wide summary statistics for the risk of CAD were obtained from a meta-analysis of CAD using data from CARDIoGRAMPlusC4D and the UK Biobank as previously described. Genome-wide summary statistics for hypertension and T2D were obtained from the IEU GWAS database using association results from the UK Biobank. Summary statistics for primary hypertension (ICD 10 code I10) as a secondary diagnosis (IEU GWAS ID “ukb-b-12493”) were used for hypertension while summary statistics for diabetes diagnosed by a doctor (IEU GWAS ID “ukb-b-10753”) were used for T2D.

A genome-wide association study to identify protein quantitative trait loci using a SomaLogic aptamer-based protein assay has previously been described. Two aptamers (SVEP1.11109.56.3 and SVEP1.11178.21.3) were used to estimate SVEP1 protein concentration. Genome-wide summary statistics for both aptamers were obtained which produced highly similar results; for simplicity, results from the analysis using the SVEP1.11178.21.3 aptamer were reported. As trans-pQTLs might affect protein levels in a variety of manners, the analysis focused on cis-pQTLs by only including variants in a 1Mb window surrounding SVEP1 which was associated with plasma SVEP1 concentration at a level exceeding genome-wide significance (P-value for SVEP1 concentration <5×10⁻⁸). These SNPs were filtered using pair-wise linkage disequilibrium estimated from the 1000 Genomes Project European samples in order to obtain an independent (r2<0.3) set of SNPs for the causal analysis. Causal estimates were calculated using the inverse-variant weighted method implemented in the R package TwoSampleMR.

### Statistical Analysis

For animal model data, a two-group independent t-test, one-way analysis of variance (ANOVA), or two-way ANOVA were used, provided the data satisfied the Shapiro-Wilk normality test. Otherwise, the Mann-Whitney U test, Kruskal-Wallis one-way ANOVA test, and Friedman two-way ANOVA test were used. Bonferroni correction was used for post hoc multiple comparison in ANOVA. Unless otherwise stated, cellular assays were analyzed by an unpaired, two-tailed t-test. Statistical analyses were performed with GraphPad Prism.

### Results

### SVEP1 is Expressed by Arterial VSMCs Under Pathological Conditions

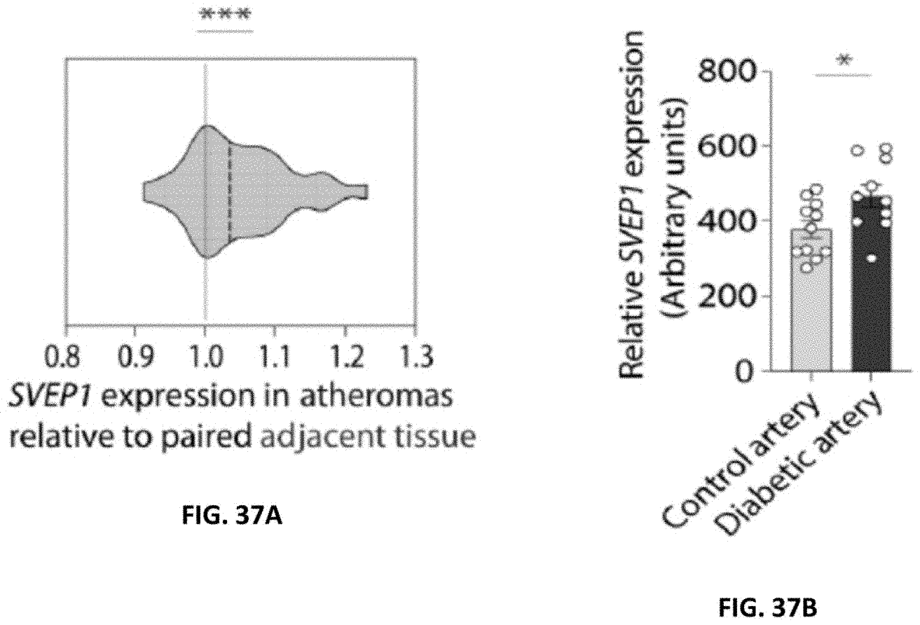

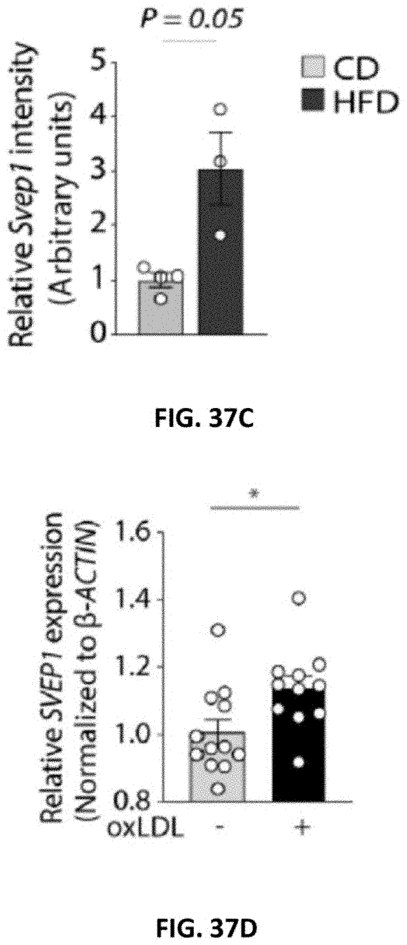

To begin characterizing the role of SVEP1 in the pathogenesis of atherosclerosis, disease-relevant tissues and cell types that express SVEP1 were sought to be identified. Expression data from the Genotype-Tissue Expression (GTEx) project indicate that human arterial tissue, including coronary arteries, expresses SVEP1. To confirm arterial expression, in situ hybridization on tissue explants from the aortic wall and internal mammary artery of patients with established coronary artery disease was used. SVEP1 expression was readily detected within cells staining with the vascular smooth muscle cell marker, smooth muscle a-actin (SMα-actin). VSMCs are known to increase the synthesis of certain extracellular matrix proteins in response to various pathological stimuli 2); therefore, expression data from relevant disease specimens were assessed to determine if this also applies to SVEP1. Indeed, SVEP1 expression is higher within human atherosclerotic tissue from carotid explants, relative to patient-paired adjacent and macroscopically intact tissue (FIG. 37A). Athero-prone arterial tissue explants from patients with diabetes also express higher levels of SVEP1 compared to patients without diabetes 2) (FIG. 37B).

Mice expressing a lacZ reporter under the native Svep1promotor of a single allele were obtained (since mice with homozygous Svep1deficiency have developmental defects and die from edema at day E18) to determine whether murine Svep1expression recapitulated human SVEP1 expression, and may therefore be a viable animal model to study its effects on disease. Within healthy arterial tissue of young mice, low β-gal expression, mostly colocalizing with VSMCs, was observed. These data are consistent with published single-cell studies that identify VSMCs within the healthy murine aorta as a minor source of Svep1expression. To determine if murine Svep1expression was increased in the development of atherosclerosis, as in humans, expression within mouse arterial tissue after the induction of atherosclerosis was assayed. Experimental atherosclerosis was induced by feeding atheroprone (Apoe−/−) mice a Western, high-fat diet (HFD) for 8 weeks. Apoe−/− mice fed a standard chow diet (CD) served as non-atherogenic controls. After 8 weeks of an atherogenic HFD, a 2-fold increase in Svep1expression relative to CD-fed control mice was observed (FIG. 37C). This expression was colocalized with neointimal cells that co-stained with SMα-actin, suggesting VSMC expression.

Numerous cell types have been demonstrated to gain expression of VSMC markers in the context of atherosclerosis. Therefore, to test the hypothesis that VSMC-derived cells within the neointima are the predominate source of SVEP1, Apoe−/− mice with VSMC-specific knockout of Svep1(Svep1flox/floxMyh11-CreERT2Apoe−/− were generated; hereafter referred to as Svep1SMCΔ/Δ) and mice with unaltered Svep1expression (Svep1+/+Myh11-CreERT2Apoe−/−; hereafter referred to as Svep1SMC+/+), which served as controls. Svep1expression was assessed using in situ hybridization within the neointima of the aortic root of both groups after 8 weeks of HFD feeding. Indeed, while robust Svep1expression in control mice was observed, neointimal Svep1expression was nearly undetectable in Svep1SMCΔ/Δ mice. These data indicate that VSMC-derived cells are the predominant source of SVEP1 in atherosclerotic plaque.

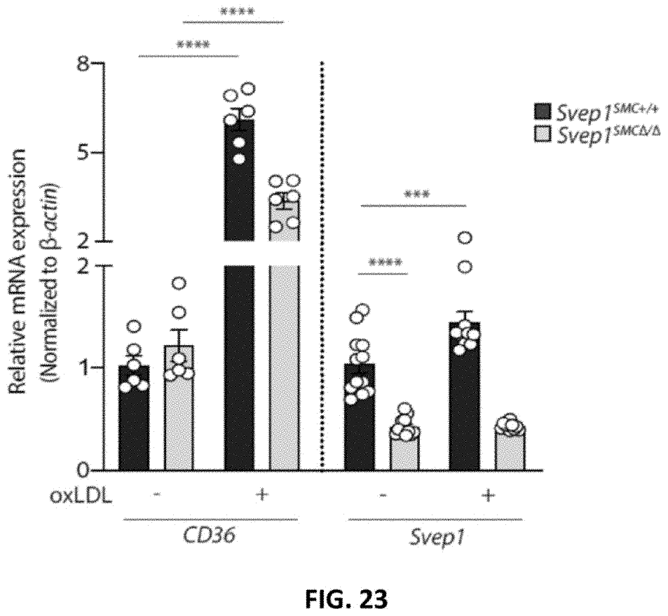

Given the increased expression of Svep1under atherosclerotic conditions in mice and humans, the ability of atheroma-associated oxidized LDL (oxLDL) to directly induce Svep1expression in VSMCs was tested. Exposure to oxLDL increased Svep1expression by 48% in primary VSMCs from Svep1SMC+/+ mice but not Svep1SMCΔ/Δ mice, compared to vehicle-treated control cells (FIG. 23). Both Svep1SMC+/+ and Svep1SMCΔ/Δ cells increased expression of CD36, indicating they were activated upon binding of oxLDL with its receptor. Exposure to oxLDL also modestly induced SVEP1 expression in human primary coronary artery smooth muscle cells (CASMCs, FIG. 37D).

Taken together, these data demonstrate that SVEP1 is produced locally by VSMCs in atherosclerotic disease and are consistent with prior studies which concluded that SVEP1 is produced by cells of mesenchymal origin. Further, these data suggest that SVEP1 may play a direct role in the pathogenesis of atherosclerosis and that mouse models are an appropriate means to interrogate this question.

### SVEP1 Drives Atherosclerotic Plaque Development

To study the effect of Svep1on atherosclerosis, Apoe−/− and Svep1+/− Apoe−/− mice were fed a HFD for 8 weeks and analyzed the resulting atherosclerotic plaque burden. There were no observed differences between genotypes in body weight, plasma total cholesterol, triglycerides, and glucose. Relative to controls, however, Svep1+/−Apoe−/− mice had a significant reduction in plaque burden (as characterized by the percentage of surface area staining positive with Oil Red O) in the aortic arch and whole aorta by en face preparations, as well as in sectioned aortic roots (FIG. 10A and 10B). The effect of Svep1haploinsufficiency on reducing the development of atherosclerotic plaque in the aortic arch and en face aorta was notably greater than in the aortic root, perhaps reflecting differences in VSMC embryonic origin and biology. Svep1haploinsufficiency also resulted in reduced macrophage staining within the aortic root neointima, as determined by the percentage of area staining positive for Mac3 (FIG. 10C). Marked differences in measures of plaque stability, such as area staining positive for VSMC markers or necrotic core size, were not observed although collagen content was modestly higher in atheromas from control mice compared to Svep1+/−Apoe−/− mice (FIGS. 14A, 14B, 14C).

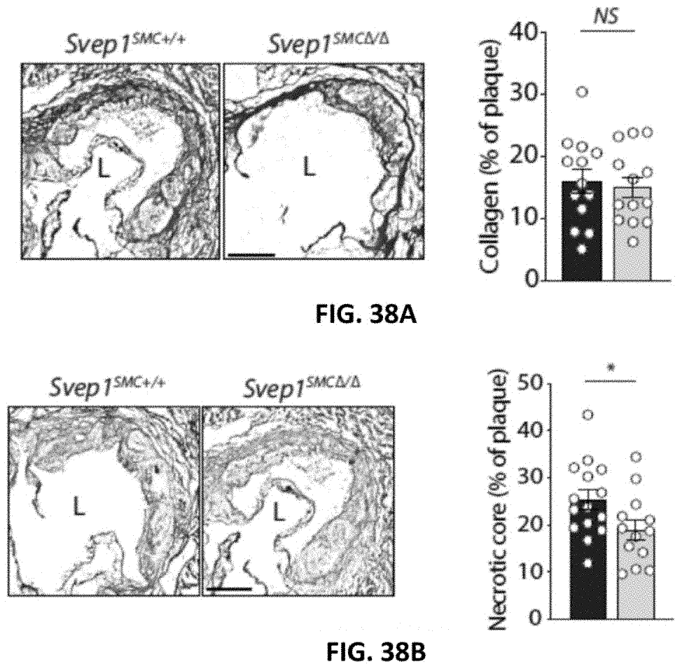

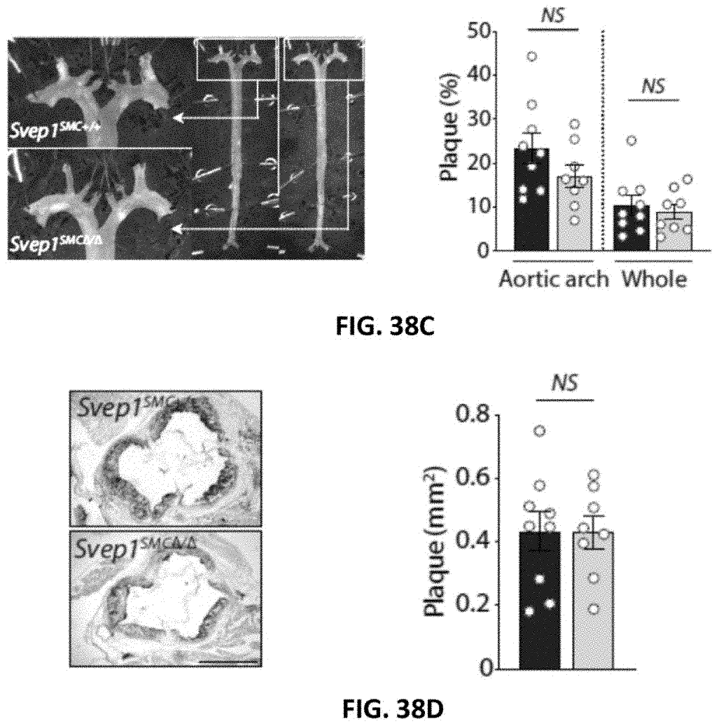

The hypothesis that the atherogenic effects of SVEP1 could be attributed to its synthesis by VSMCs using Svep1SMCΔ/Δ and Svep1SMC+/+ mice, as previously described, was tested. As with Svep1haploinsufficiency, loss of Svep1in VSMCs did not significantly alter body weight, plasma cholesterol, triglycerides, and glucose levels following 8 weeks of HFD feeding. Also consistent with our Svep1haploinsufficiency model, Svep1SMCΔ/Δ mice had decreased plaque burden plaque in the aortic arch, whole aorta, and aortic root, as compared to Svep1SMC+/+control mice. Additionally, atheromas from Svep1SMCΔ/Δ mice contained less macrophage staining and necrotic core area, indicators of plaque instability, and unaltered collagen content (FIGS. 12A, 38A, 38B).

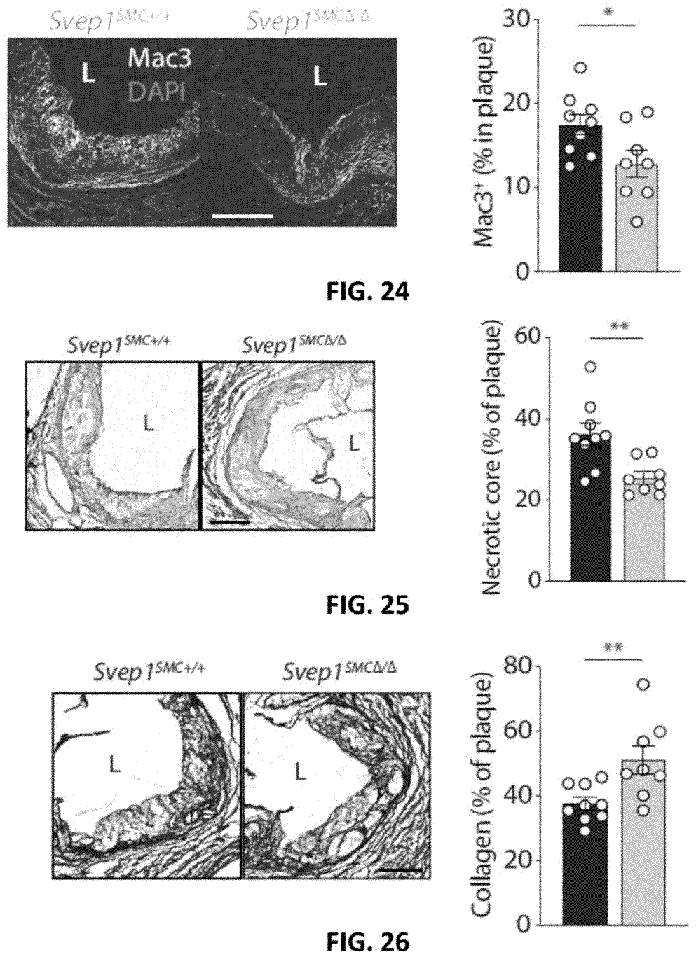

Given the observations that loss of Svep1in VSMCs resulted in a dramatic reduction in plaque size in the setting of 8 weeks of HFD feeding, the length of plaque development to investigate the effect of SVEP1 on advanced plaque lesions was extended. After treatment with tamoxifen, Svep1SMC+/+ and Svep1SMCΔ/Δ mice were fed HFD for 16 weeks. Again, no differences were observed in body weight, plasma cholesterol, and glucose levels between the groups. Triglycerides were higher in the Svep1SMCΔ/Δ mice at a level of nominal significance (P=0.046), but this was not observed at other timepoints or in the haploinsufficiency model. Although a statistically significant effect of VSMC-specific Svep1deletion on atherosclerotic plaque burden was not detected (FIG. 38C), plaques from Svep1SMCΔ/Δ mice tended to be smaller and were both less complex and more stable than controls. These indicators of an altered plaque phenotype include decreased neointimal macrophage staining (FIG. 24) and necrotic core size (FIG. 25), in addition to greater collagen content (FIG. 26). Taken together, these experimental atherosclerosis data suggest that SVEP1 drives atherosclerosis and increases plaque complexity in mice.

### SVEP1 is Causally Related to Cardiometabolic Disease in Humans

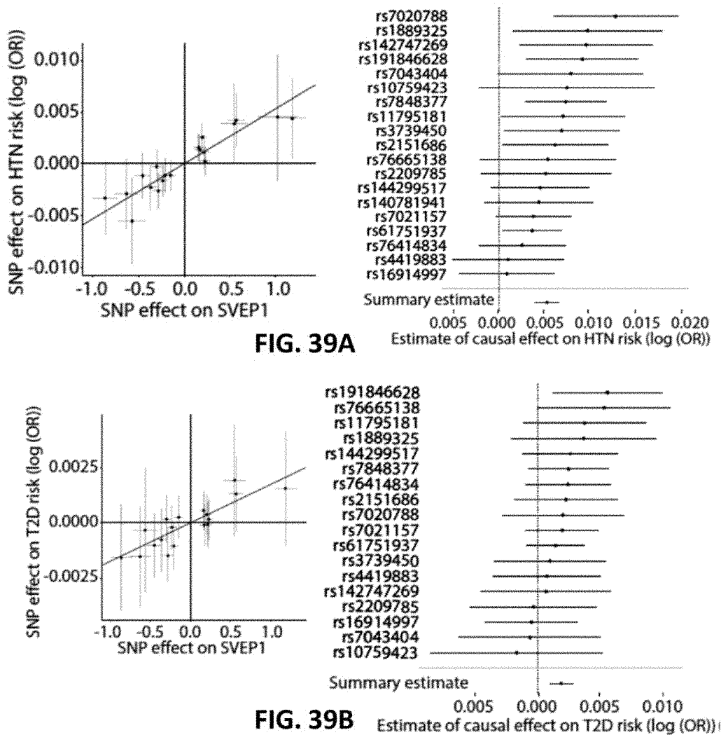

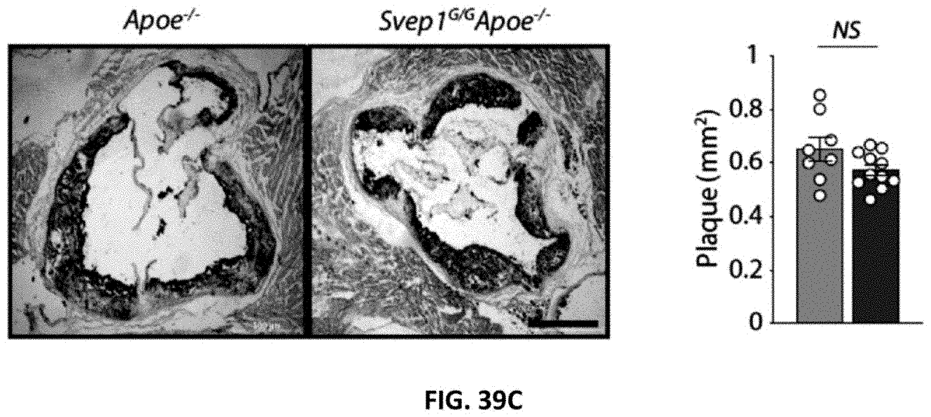

Due to the relationship that was discovered between SVEP1 depletion and reduced atherosclerosis across our mouse models, it was questioned whether the human SVEP1 CAD-associated D2072G missense polymorphism was associated with altered SVEP1 levels in humans. While it was not found that this allele (or other alleles in linkage disequilibrium) were associated with mRNA levels in GTEx, it was found that the 2702G risk variant (SVEP1CADrv) was associated with a significant increase in circulating plasma SVEP1 protein levels (P=8×10⁻¹⁴; FIG. 27A) as measured by two independent aptamers from participants in the INTERVAL study, suggesting that increased SVEP1 protein levels were associated with increased risk of CAD. Then other genetic variants influencing SVEP1 protein levels were tested. Using published data from the INTERVAL study, cis-acting variants that associated with SVEP1 protein levels at a genome-wide (P<5×10⁻⁸) level of statistical significance were cataloged (FIG. 27B). Mendelian Randomization was performed using a subset of these variants in linkage equilibrium (r2<0.3) and it was found that increased SVEP1 protein levels were causally related to increased CAD risk (P=7×10¹¹; FIG. 27C). It was also asked if SVEP1 protein levels were causally related to increased risk for hypertension and type 2 diabetes due to the prior associations we observed for the SVEP1CADry allele with these risk factors. Indeed, it was found that increased SVEP1 protein levels were causally related to both hypertension (P=2×10⁻¹⁵; FIG. 39A) and type 2 diabetes (P=0.0004; FIG. 39B).

To investigate how the human SVEP1CADry missense polymorphism might impact CAD risk, homozygous mice harboring the human SVEP1CADry at the orthologous murine position (Svep12699G/2699G; hereafter referred to as Svep1G/G) were generated. These mice were bred with Apoe−/− mice to generate Svep1G/GApoe−/− mice. Differences in body weight, serum total cholesterol, triglycerides, and glucose between groups after feeding HFD were not able to be detected. A significant difference between groups in the development of atherosclerotic plaque at either 8 or 16 weeks of HFD feeding was not detected (FIG. 39C). Although the prior human genetic study revealed a significant association with an increased risk of CAD, the effect of the SVEP1CADry in humans was modest, in which each copy of the G allele was associated with a 14% increased risk of disease. If an effect size in mice is similarly modest, further investigation would require a very large number of animals, presenting both pragmatic and ethical barriers. To circumvent these concerns, subsequent functional interrogation of the SVEP1CADry was performed in vitro.

### VSMCs Express Integrin α9β1

To begin characterizing the mechanism by which SVEP1 drives atherosclerosis, receptors and associated cell types that interact with SVEP1 in the extracellular space were attempted to be identified. Integrin α9β1 is the only protein known to interact with SVEP1 and they colocalize in vivo. Integrins are transmembrane, heterodimeric receptors that respond to the extracellular environment and influence numerous aspects of atherosclerosis. Therefore, it was hypothesized that integrin α9β1 (and associated cell-types) may be involved in SVEP1-mediated atherosclerosis. The α9subunit (ITGA9) is known to exclusively heterodimerize with (31 (ITGB1), therefore assessing ITGA9 expression is a reliable proxy for integrin α9β1 expression. Integrin α9β1 expression has been documented in airway epithelium, smooth muscle, skeletal muscle, hepatocytes, and epithelial cells, yet arterial tissue expresses the highest ITGA9 levels of all GTEx tissues. In situ hybridization confirmed that ITGA9 is broadly expressed in the human aortic wall and LIMA, predominately colocalizing with VSMCs. Likewise, VSMCs of the murine aorta expressed high levels of Itgα9. Consistent with these data, single-cell studies of the murine aorta indicate the VSMCs express Itgα9. Given the established role of VSMCs in CAD, their expression of integrin α9β1, and the local expression patterns of SVEP1 in disease, the hypothesis that VSMCs respond to SVEP1 in a cell-autonomous manner to promote atherosclerosis was tested.

### SVEP1 Induces Proliferation and Integrin Signaling in VSMCs

The ECM plays a critical role in orchestrating cellular responses to tissue injury, including promoting cell proliferation and differentiation. Therefore, the proliferation of neointimal Svep1SMCΔ/Δ and Svep1SMC +/+ VSMCs using immunofluorescent staining of the proliferation marker, mini-chromosome maintenance protein-2 (MCM-2), was assessed. Among cells expressing smooth muscle actin, fewer stained positive for MCM-2 in Svep1SMCΔ/Δ mice as compared to Svep1SMC +/+ controls after HFD feeding for 8 weeks (FIG. 13A), suggesting SVEP1 induces VSMC proliferation.

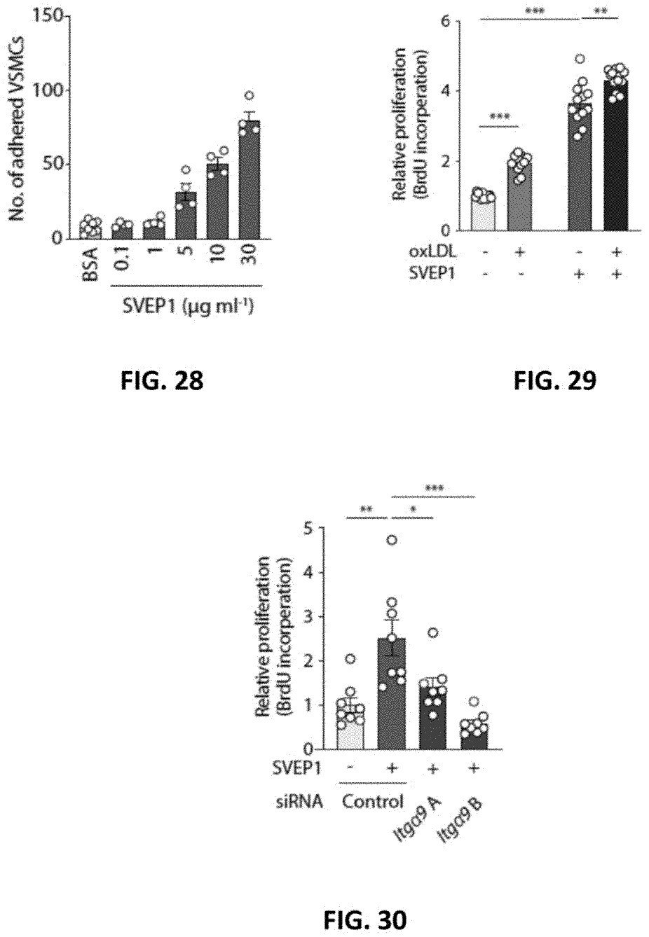

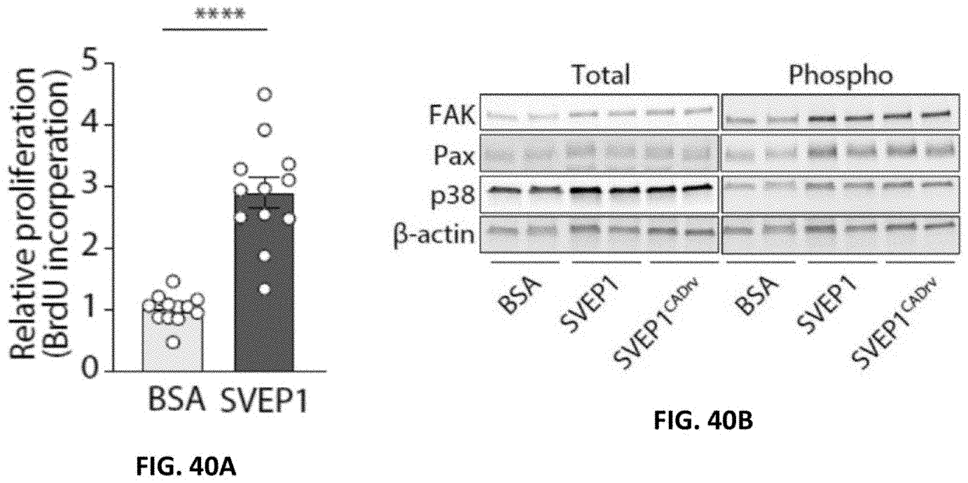

To further explore the effects of SVEP1 on VSMCs, recombinant SVEP1 and its orthologous CAD risk variant (SVEP1CADrv) using a mammalian expression system was generated and purified. The response of primary VSMCs to SVEP1 that was immobilized on culture plates was tested, reflecting an overexpression-like assay while maintaining its physiologic context as an extracellular matrix protein (in contrast to genetic overexpression). VSMCs adhere to SVEP1 in a dose-dependent manner (FIG. 28). Exposure to both SVEP1 variants induces dose-dependent VSMC proliferation, based on BrdU incorporation (FIG. 18). As a point of reference, oxidized low-density lipoprotein (oxLDL) was used, a proliferative stimulus relevant to atherosclerosis, in addition to SVEP1 to test VSMC proliferation. Strikingly, SVEP1 was able to induce more VSMC proliferation than oxLDL. Exposure to a combination of oxLDL and SVEP1, as exists within the atheromatous environment, caused the greatest amount of VSMC proliferation (FIG. 18). Human coronary artery smooth muscle cells (CASMCs) also proliferate in response to SVEP1 (FIG. 40A). Murine macrophages exposed to SVEP1 do not proliferate in the absence or presence of oxLDL (FIG. 28) suggesting that SVEP1 is not a proliferative stimulus for all cell types.

Integrin α9β1 is expressed by VSMCs, binds to SVEP1, and drives proliferation in some cell types. Therefore, to begin to interrogate the molecular mechanisms by which SVEP1 influences VSMCs, whether SVEP1 exposure was able to induce integrin signaling in VSMCs was tested. This was tested by seeding cells to wells coated with bovine serum albumin (as an inert protein control), VCAM-1 (a low-affinity integrin α9β1 ligand), or SVEP1 (a high-affinity integrin α9β1 ligand). It was found that cells adherent to SVEP1 had increased phosphorylation of canonical integrin signaling kinases, such as focal adhesion kinase (FAK), Paxillin (Pax), and Src, as well as downstream MAPK kinases, ERK and p38 (FIG. 29), relative to an inert protein control. SVEP1CADry had similar effects as SVEP1 on integrin signaling in VSMCs (FIG. 40B). It was then tested whether SVEP1-induced proliferation was dependent on integrin α9β1. Since Itgα9 exclusively heterodimerizes with Itg01, siRNA knockdown of Itgα9 to disrupt integrin α9β1 was used. The proliferative effect of SVEP1 was completely inhibited by the knockdown of Itgα9 using two different siRNA constructs (FIG. 30), suggesting that integrin α9β1 is necessary for SVEP1-induced VSMC proliferation.

### SVEP1 Regulates Key VSMC Differentiation Pathways

Next, the response of primary VSMCs to the wildtype SVEP1 and SVEP1CADry proteins was characterized using an unbiased methodology. Cells were collected after 20 hours of growth on the indicated substrate and transcriptomic analysis was performed using RNA-sequencing. Pathway and gene ontology analysis was used to determine the shared and unique transcriptional response to the SVEP1 variants. Consistent with previous findings, a number of cell adhesion and proliferation-related pathways and terms were enriched in the shared transcripts of cells exposed to either SVEP1 variant. These include ECM-receptor interaction, focal adhesion, integrin-mediated signaling, positive regulation of cell proliferation, and various additional proliferative and mitogenic pathways. A striking number of differentiation and development-related pathways and terms were also enriched in cells exposed to the SVEP1 variants. These include angiogenesis, cell differentiation, and wound healing, among many others.

SVEP1 contains numerous different and repeating domains that are known to play critical developmental roles and may therefore be governing the effects of SVEP1 on VSMCs. Further, although Svep1−/− and Itgα9−/− mice have similar phenotypes of edema and lymphatic defects, the phenotype of Svep1−/− mice is markedly more severe (death by E18.5 vs P12), suggesting that ITGA9 may have partial redundancy with an additional receptor(s) for SVEP1. To search for evidence of additional domain interactions, the transcriptional profile of VSMCs to the SVEP1 variants with InterPro 2, a database of protein domains, was cross-referenced. In addition to integrin-related domains, transcripts that code for EGF-like domain-containing proteins were highly differentially expressed in cells exposed to SVEP1. Repeat EGF-like domains often interact, as occurs in Notch signaling, suggesting SVEP1's repeat EGF-like domains may be playing an important, but as of yet undescribed role in the biological function of SVEP1. Indeed, transcripts related to Notch signaling were dysregulated in cells exposed to SVEP1.

As an orthogonal approach to interrogating SVEP1's mechanisms and potential binding partners, homologs in distantly related species were attempted to be identified. The Drosophila protein, uninflatable, is a potential orthologue of SVEP1 and contains a region defined by three ephrin-receptor-like domains, followed by tandem EGF-repeats and a Laminin-G domain, mirroring a region of SVEP1 that contains a highly similar sequence of domains. Inhibition of uninflatable in Drosophila larvae results in defective tracheal development, analogous to the vascular defects observed in zebrafish Svep1mutants. Uninflatable has been shown to bind and modulate Notch signaling in Drosophila. These findings, in addition to the RNAseq analysis, led us to hypothesize that SVEP1 may also modulate Notch signaling.

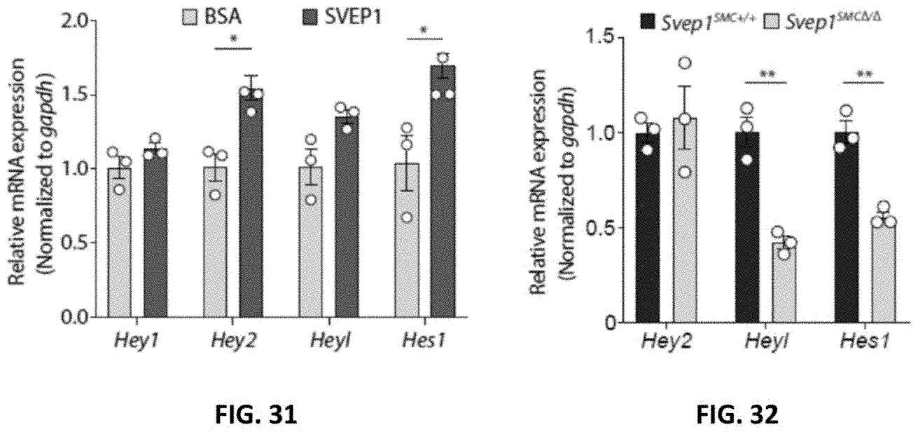

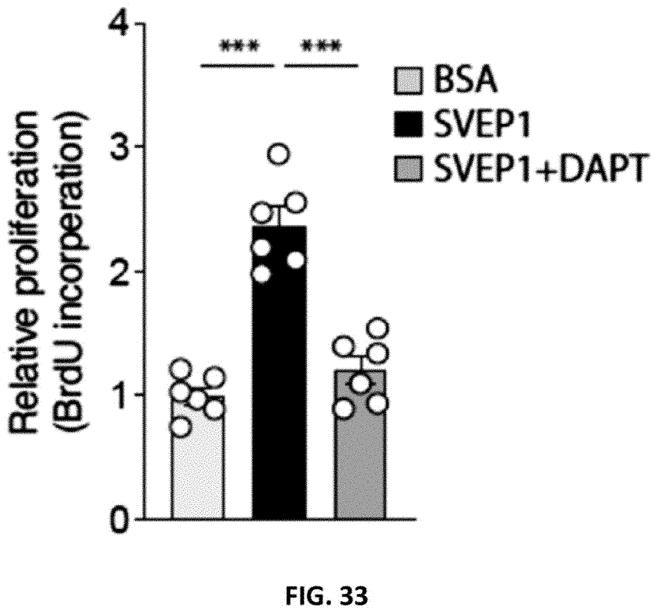

VSMCs express multiple Notch receptors, thus, the impact of SVEP1 on Notch signaling in VSMCs was tested. This was assessed by seeding VSMCs on tissue culture plates treated with SVEP1 or BSA (as an inert control protein) for 4 hours, since Notch signaling is highly temporally regulated. Cells grown on SVEP1 had significantly increased expression of canonical Notch targets Hey2 and Hes 1 even without overexpression of a Notch receptor (FIG. 31). Conversely, primary VSMCs collected from Svep1SMCΔ/Δ mice have decreased transcription of Notch target genes (FIG. 32), supporting the regulation of Notch signaling by SVEP1. SVEP1-induced proliferation was also completely abrogated upon Notch inhibition by the y-secretase inhibitor, DAPT (FIG. 33). Cell proliferation in response to 10% fetal bovine serum was not significantly inhibited by DAPT, demonstrating that Notch signaling is necessary for SVEP1-induced proliferation. It is possible that Notch and integrin receptors may cooperatively regulate the effects of SVEP1, similar to that reported on non-canonical ECM Notch regulators MAGP2 and EGFL7.

### VSMCs Differentially Respond to SVEP1^{CADrv} Compared to SVEP1

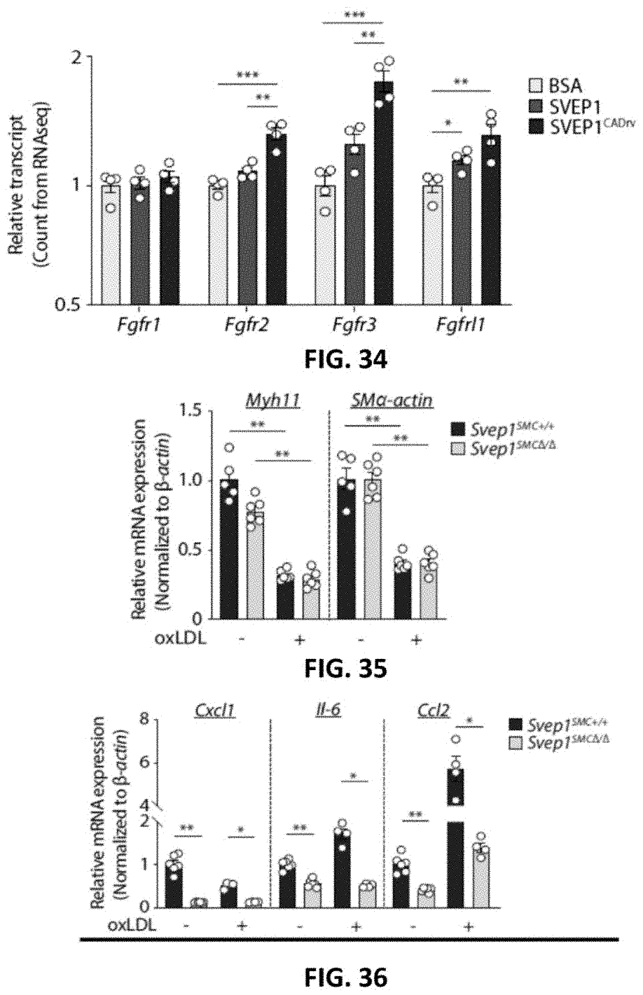

Experimental atherosclerosis models and Mendelian Randomization analysis indicate that both SVEP1 variants are atherogenic, with SVEP1CADry having the greater atherogenicity of the two. Therefore, the differential transcriptional responses of VSMCs to the SVEP1 variants were interrogated. This analysis also revealed that a large number of proliferation-related pathways were disproportionately regulated by the variants. Further exploration revealed that the fibroblast growth factor (FGF) receptor family was differentially expressed between the variants. The FGFR family is also sub-categorized within several of the most differentially regulated pathways and terms. FGF signaling is pro-atherogenic in VSMCs, so the effect of each variant on the direction and magnitude of transcription of each FGF receptor expressed by VSMCs was assessed. Consistent with their relative atherogenicities, SVEP1 increases expression of FGF receptors but exposure to SVEP1CADry resulted in significantly higher expression of FGF receptors (FIG. 34). These data suggest that increased FGF signaling may contribute to the increased CAD risk associated with SVEP1CADrv.

Given the fundamental role of integrin, Notch, and FGFR signaling in regulating VSMC phenotype, the effects of SVEP1 in response to oxLDL, an inflammatory stimulus relevant to atherosclerosis, was assessed. Upon oxLDL stimulation, both Svep1SMC+/+ and Svep1SMCΔ/Δ VSMCs decreased the expression of contractile markers Myh11 and SMα-actin (FIG. 35), while increasing expression of the inflammatory markers I1-6 (interleukin-6) and Ccl2 (C-C motif chemokine ligand 2) (FIG. 36), confirming an inflammatory response to oxLDL. Cxcl1 (C-X-C motif chemokine ligand 1), I1-6, and Ccl2 expression were lower in Svep1SMCΔ/Δ VSMCs than Svep1SMC+/+controls, suggesting that SVEP1 may be a pro-inflammatory stimulus in VSMCs under atherosclerotic conditions.

### SVEP1 Promotes Inflammation in Atherosclerosis

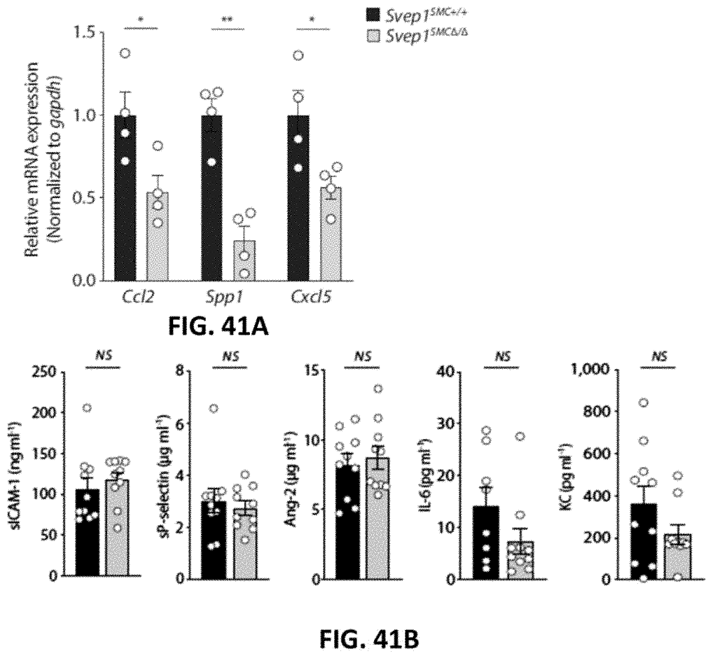

To investigate how the loss of Svep1influences pathways involved in the development of atherosclerosis at the tissue level, RNA-seq analyses on mRNA extracted from aortic arches of Svep1SMC+/+ and Svep1SMCΔ/Δ mice after 8 weeks of HFD were performed. Loss of Svep1in VSMCs altered a number of inflammatory pathways upon induction of atherosclerosis. These include cytokine-cytokine receptor interaction, chemokine signaling, and NF-kappa B signaling pathways. Notably, both cell adhesion molecules (CAMs) and ECM-receptor interaction were also dysregulated in the atherosclerotic aortic arches from Svep1SMCΔ/Δ . Quantitative PCR using cDNA from the aortic arches of the same mice was used to validate the RNA-seq results. Specifically, Cc12, Sppl (secreted phosphoprotein 1, also known as osteopontin), and Cxcl5 (C-X-C motif chemokine ligand 5) were significantly decreased in Svep1SMCΔ/Δ mice, as compared to Svep1SMC+/+ mice (FIG. 41A). Despite these differences, a significant alteration in circulating inflammatory mediators in these mice was not found, suggesting SVEP1 influences local tissue inflammation but not systemic inflammation (FIG. 41B). These data are also consistent with our observations that Svep1depletion decreases neointimal macrophage staining in atherosclerotic plaque.

Integrins play a critical role in the immune response, therefore, it was asked whether immune cells may also express integrin α9β1 and interact with SVEP1 in atherosclerosis. In human peripheral blood cells, moderate integrin α9β1 expression was detected by neutrophils and low expression was detected by CD14lowCD16+ non-classical, CD14highCD16+ intermediate, and CD14+CD16-classical monocytes as previously reported. Given that monocytes significantly alter their expression profiles upon tissue entry and differentiation into macrophages, whether macrophages in atherosclerotic plaque express ITGA9 was tested. Indeed, ITGA9 expression was detected in CD68+macrophages within human atherosclerotic plaque by in situ hybridization.

The expression of integrin α9β1 expression in circulating murine leukocyte subsets was assessed. High expression of integrin α9β1 was detected in both Ly6Chi and Ly6Clow monocytes and we could detect low levels in neutrophils. These expression patterns were unaltered in heterozygous Svep1deficiency and an induction of integrin α9β1 expression upon oxLDL treatment in any cell type tested was not observed. Considering the finding that integrin α9β1 is expressed by monocyte subsets in peripheral mouse blood, its expression in myeloid cells from the aortas of Apoe−/− and Svep1+/−Apoe−/− mice following 8 weeks of HFD feeding was further analyzed. It was discovered that integrin α9β1 was expressed in both macrophages and Ly6Chi monocytes of these mice, consistent with human expression data. Robust expression of Itgα9 by neointimal macrophages using in situ hybridization was similarly detected.

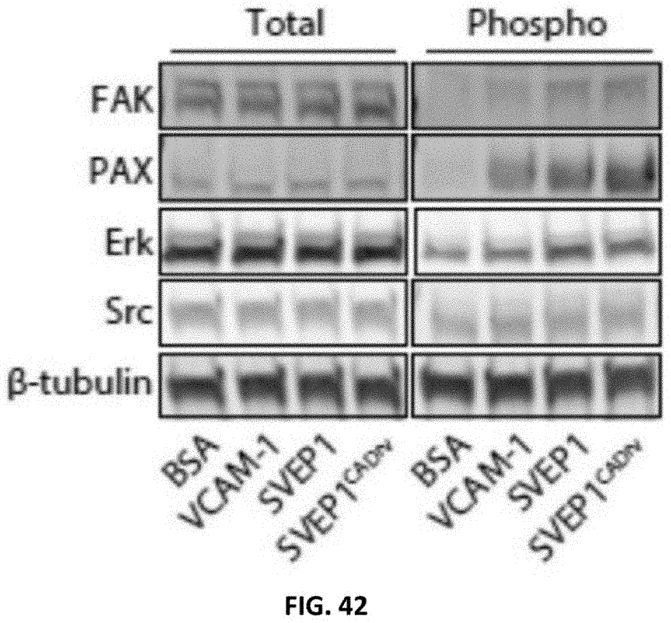

Since integrin α9β1 is expressed on monocytes/macrophages, whether SVEP1 could be directly interacting with integrin α9β1 on these cells was interrogated. To test this, mice with myeloid cell lineage-specific knockout of Itgα9 using LysM-Cre (Itgα9 flox/floxLysM-Cre, hereafter referred to as Itgα9 MACΔ/Δ) were generated. Itgα9+/+ LysM-Cre mice, referred to as Itgα9 MAC+/+, served as controls. First, bone marrow-derived macrophages from Itgα9 MACΔ/Δ animals had a significant reduction in the amount of integrin α9β1 that was present on the cell surface. The ability of peritoneal macrophages from these animals to migrate in response to SVEP1 was tested using a trans-well migration assay. SVEP1 exposure induced a dose-dependent trans-well migration of macrophages from Itgα9MAC+/+control animals but not from Itgα9MACΔ/Δ mice (FIG. 12D). This suggests that SVEP1 and integrin α9β1 may directly interact to augment myeloid cell homing or migration. Consistent with this, THP-1 cells, a human monocytic cell line, adhered to SVEP1 in a dose-dependent manner. Integrin signaling was also activated in THP-1 cells upon exposure to SVEP1 or SVEP1CADry and no differences were observed between the variants (FIG. 42).

To test if SVEP1 had similar effects on leukocytes in vivo, an in vivo monocyte recruitment assay was performed in Svep1SMC+/+ and Svep1SMCΔ/Δ mice. After 8 weeks of HFD feeding, yellow/green (YG) latex beads were injected intravenously in order to label circulating Ly6Clow monocytes. Flow cytometry was performed three days after intravenous bead injection (to confirm labeling) and the aortic tissues were isolated for histology on the fourth day following bead injection (to assess recruitment). It was confirmed that YG beads were preferentially labeled on Ly6Clow monocytes and not on Ly6Chigh monocytes, indicating efficient bead labeling of circulating monocytes. A difference between groups in the efficiency of bead labeling for monocyte subsets was not observed. Next, the number of labeled monocytes recruited into atherosclerotic plaques of aortic roots was quantified using fluorescent microscopy. Svep1SMCΔ/Δ mice had significantly fewer YG beads per atheroma, with or without normalization to the percentage of labeled monocytes, relative to Svep1SMC+/+ mice (FIG. 12C).

Taken together, these data support SVEP1's role in promoting inflammation in atherosclerosis, either indirectly by promoting an inflammatory VSMC phenotype, directly by interacting with integrin α9β1 on circulating or tissue leukocytes, or a combination of these processes.

### Discussion

Human genomic studies hold great promise in identifying therapeutic targets for disease, but a significant limitation in translating their findings is the identification of specific causal genes that underlie the observed statistical associations. In a previous study, a low-frequency polymorphism in SVEP1 that was robustly associated with coronary artery disease risk in humans was identified, but it was not clear if SVEP1 was the causal gene in the locus. Here, the first report that SVEP1 is causal in coronary artery disease is presented using experimental mouse models and Mendelian Randomization.

Atherosclerosis is a complex, multifactorial disease process with numerous cell types playing a role in its pathogenesis. This presents an arduous challenge when validating genomic risk loci and testing their mechanisms. The SVEP1CADry does not associate with changes in plasma lipid levels, prompting us to explore how SVEP1 might influence other aspects of disease pathogenesis. Human and mouse expression data at the cell and tissue level was used to develop mechanistic hypotheses, which were then tested using in vivo and in vitro approaches. Specifically, high basal arterial expression of both SVEP1 and ITGA9, and increased SVEP1 expression under pathological conditions, led to the hypothesize that these proteins may influence local disease processes. Upon exposure to various pathologic stimuli, VSMCs can undergo a “phenotype shift”, in which they lose their quiescent, contractile properties and become migratory, proliferative, inflammatory, and synthetic. VSMCs gain properties of matrix-synthesizing fibroblasts during atherosclerosis, making VSMCs our primary candidates for the source of SVEP1 within atherosclerotic plaque. Consistent with prior reports which concluded that SVEP1 is not produced by endothelial or immune cells, negligible Svep1expression in the plaques of Svep1SMCΔ/Δ animals was observed. Thus, the results provide strong evidence that atherogenic SVEP1 is indeed synthesized by VSMC-derived cells within the atherosclerotic plaque.

Using two independent mouse models in which Svep1was depleted either partially in all cells (Svep1SMC+/−) or fully in only VSMCs (Svep1SMCΔ/Δ), depleting SVEP1 resulted in a significant reduction in the development of atherosclerotic plaque with a magnitude of effect similar to murine models of other CAD risk loci. Then the expression of ITGA9 was used to identify disease-relevant cell types that may respond to SVEP1. This led to the hypothesis that SVEP1 may be interacting with VSMCs by an autocrine mechanism or monocytes by a paracrine mechanism to promote atherosclerosis. VSMCs play a particularly complex and intriguing role in atherosclerosis and warrant further discussion. Recent lineage tracing studies have challenged the notion that VSMCs play a protective role in atherosclerosis by demonstrating that a large, heterogenous population of cells within plaque are derived from VSMCs. Furthermore, numerous CAD risk loci have now been linked to VSMCs. This study demonstrates that SVEP1 profoundly influences the behavior of VSMCs by regulating a number of pathways with vital roles in VSMC biology. These pathways include integrin, Notch, and FGFR signaling, each of which has been shown to contribute to atherosclerosis. Recent studies have provided novel insights into the regulation of VSMC phenotype in atherosclerosis by various transcription factors. The ECM also plays a fundamental role in regulating VSMC phenotype and is amenable to pharmacologic intervention. Current strategies for the treatment and prevention of CAD consist of lowering risk factors, such as plasma lipids, yet substantial residual risk remains despite effective treatment. Intervening on VSMCs may be a powerful complimentary approach to these traditional therapies.

In addition to its association with CAD, the Mendelian randomization results suggest that circulating SVEP1 causally underlies the risk of hypertension and type 2 diabetes. Although the source of SVEP1 in human plasma is unknown, other ECM proteins have been detected in the circulation of patients with atherosclerosis, suggesting that plasma levels of these proteins may reflect tissue levels and atherosclerotic remodeling. The mechanisms by which the genetic variants used in the Mendelian randomization affect plasma SVEP1 levels are unclear. Two reasonable hypotheses include modification of protein secretion or degradation. Alternatively, it is possible that protein-modifying polymorphisms could alter the affinity of aptamer binding (and thus impact the estimated plasma protein levels). It is unlikely that altered aptamer binding is a significant contributor, however, because differential plasma SVEP1 levels were observed using two independent aptamers and because the majority of genetic variants linked to plasma levels were not located in protein-coding DNA segments. Further studies will be required to determine the precise mechanisms by which these variants affect plasma protein levels. Regardless, the power of the two sample Mendelian randomization framework is that these alleles are allocated randomly at birth and are associated with SVEP1 levels in the absence of disease, suggesting that the presence of disease is not driving altered SVEP1 levels, but rather that altered SVEP1 levels are causally related to disease. This further suggests that circulating SVEP1 levels have the potential to be useful as a predictive biomarker.

Additional human genetic data support a broader role of SVEP1 in cardiometabolic disease. The alpha subunit of integrin α9β1, which binds to SVEP1 with an affinity that far exceeds its other known ligands, is also associated with blood pressure in multiple studies. Overexpression of disintegrin and metalloproteinase with thrombospondin motifs-7 (ADAMTS-7), another CAD risk locus, in primary rat VSMCs alters the molecular mass of SVEP1. The overlapping disease associations and molecular interactions between these three risk loci converge on SVEP1 and point to a regulatory circuit with a prominent, yet unexplored role in cardiometabolic disease. Further studies will be required to validate their interactions and mechanisms in vivo, and to explore the potential of targeting this pathway for the treatment of cardiometabolic disease.

The complementary mouse models demonstrate that Svep1haploinsufficiency and VSMC-specific Svep1deficiency significantly abrogate the development of atherosclerosis. Each intervention was well tolerated by mice, as we did not observe any adverse response to SVEP1 depletion. Similarly, the Mendelian Randomization analyses suggest there may be a therapeutic window to safely target SVEP1 levels. These findings suggest that targeting SVEP1 or selectively modulating its interactions may be a viable strategy for the treatment and prevention of coronary artery disease.

### Example 8: SVEP1 and Pearl 1 Interact to Promote AKT/mTOR Signaling and Platelet Reactivity

### Methods

Study approval

Blood collection from consenting healthy controls was conducted in accordance with the 430 Institutional Review Board of Washington University, St Louis. All animal studies were performed according to procedures and protocols approved by the Animal Studies and Institutional Animal Care and Use Committees of the Washington University School of Medicine.

### Statistics

The specific statistical methods used to analyze each set of data are described in the figure legends and/or the specific methods section. The paired data were analyzed by a two-tailed, paired t-test. The unpaired data were analyzed by a two-tailed, unpaired t-test or a two-way analysis of variance (ANOVA) if another variable, such as sex, was a potential source of variation. One-way ANOVA was used when making multiple comparisons to a single reference group, followed by the indicated statistical test. Individual data points were shown whenever possible, however, least squared means were used to simplify data visualization in limited cases. Data were excluded prior to analysis whenever a technical error was noted during data collection. Extreme outliers were excluded from the mouse hematological studies using the ROUT method under the most stringent threshold (Q=0.1%). The cell culture and molecular data included in this manuscript are representative of at least two independent experiments. The BLI assay was performed three independent times with similar results. The animal experiments were performed at least once. The protein array experiments served as a screening tool and were performed once. Excluding the indirect calorimetry measurements, the animal experiments were performed in a blinded and randomized fashion. The cellular studies, molecular studies, and data analysis were performed in an unblinded fashion. Densitometry of immunoblots was performed using Image Lab and reported whenever the results were not abundantly clear. The data were analyzed and graphed in GraphPad PRISM 8 or 9. The data panels were imported and formatted into figures using Adobe Illustrator. Stars were used to denote statistical significance in the functional studies. *P<0.05, **P<0.01, ***P<0.001.

### Mendelian Randomization

Genome-wide summary statistics for plasma levels of SVEP1 (aptamer SVEP1.11109.56.3 and aptamer SVEP1.11178.21.3) and Pearl 1 (aptamer Pearl 1.8275.31 chosen for their ability to detect Pearl 1 cis-pQTLs) were obtained from the previously published INTERVAL and deCODE studies in 3,301 European and 35,559 Icelandic individuals, respectively. Unlinked GWAS markers (r2≤0.2) from deCODE (as estimated from 1000G European sequence data) were used to generate instrumental variables for plasma levels of SVEP1 and Pearl 1. As trans-pQTLs may affect protein levels in a variety of manners, analysis on cis-pQTLs by only including variants in a 250kb window surrounding the gene of interest and associated with altered plasma levels of the associated protein at a level exceeding genome-wide significance (P-value for respective plasma protein concentration ≤5×10⁻⁸) was focused on.

The reported outcomes included plasma levels of proteins from INTERVAL, platelet traits, and CAD. For blood platelet traits, previously published summary statistics for platelet count and mean platelet volume in Europeans were obtained. Summary statistics for CAD were obtained from the previously published meta-analysis of UK Biobank and CARIDoGRAMPlusC4D. Causal analysis was performed using the inverse-variant weighted method implemented in the R package TwoSampleMR.

### Biolayer Interferometry

BLI assays were performed on the ForteBio Octet RED96e and analyzed using Octet Data Analysis HT 12.02.2.29 software. Biotinylated Pearl 1ECD, described subsequently, was loaded on Sartorius Octet SA Biosensors. Loading was performed in the presence of 1% BSA and was stopped after all sensors reached a 1.5nm shift. The sensors were then quenched with 5 mg/mL biocytin. After establishing a baseline in assay buffer (0.01% Tween 20 in calcium-containing DPBS), the sensors were placed in solutions of SVEP1 ranging from 2.5-80 nM. A solution containing 0 nM SVEP1 was used as a buffer reference. After 15 minutes of association, the sensors were returned to the assay buffer for 15 minutes of dissociation. Sensors lacking Pearl 1 did not respond to SVEP1 (sensor reference). The original sensograms were negative and flipped for analysis. Solutions containing anti-Pearl 1 pAb also resulted in a negative sensogram in Pearl 1ECD-loaded sensors. The buffer reference was subtracted from the sensograms prior to quantification. Savitzky-Golay filtering was applied to the data. The following inter-step corrections were applied: association step (1-3 seconds after the start of the association), and dissociation step (5 seconds after the start of dissociation). The sensograms were fit to a global, 1:1 binding curve, R2=0.999. Local fittings provided similar dissociation constants (1.43-9.90 nM), compared to the global fitting (8.78 nM).

### Immunoprecipitation

For the Pearl 1 immunoprecipitation (IP): 150 μg of cell lysates from human platelets exposed to immobilized BSA, SVEP1, or a soluble polyclonal Pearl 1 antibody for 15 minutes were incubated with 2 μg Pearl 1 monoclonal antibody (R&D Systems) for 2 hours. Subsequently, 204, of BSA-blocked Invitrogen Protein A Dynabeads slurry was added, followed by rotation for 45 minutes. The beads were separated using magnetism and washed with RIPA buffer 4-5 times, then resuspended with reducing LDS sample buffer and analyzed by immunoblot assay. Bands for p-tyrosine and Pearl 1 were detected at ˜140 kDa.

For the SVEP1/Pearl 1 Co-IP: Recombinant SVEP1-Myc and Pearl 1ECD-Bio-His or Pearl 1ECD (R&D Systems) were added to assay buffer in microcentrifuge tubes in a 3:1 mass ratio, resulting in an approximately equal molar ratio. The assay buffer consisted of lmg/mL BSA, 0.01% Tween 20, and 10 mM Ca²⁺ in PBS. The proteins were incubated together for two hours while rotating. Where indicated, 2 μg primary antibodies were added during the final 30 minutes of the initial incubation. Following the incubation, aliquots were reserved as the input fraction. Subsequently, 6-10 μL slurry of Pierce Anti-c-Myc Magnetic Beads, Invitrogen Dynabeads Protein G beads, or Pierce Streptavidin Magnetic Bead slurry were added, followed by a 1-hour incubation at 20° C. with gentle agitation. Beads were washed in PBS +0.01% Tween 20 and resuspended reducing LDS sample buffer and analyzed by immunoblot assay. Bands for SVEP1 were detected at approximately 300 kDa. Bands for Pearl 1ECD were detected at approximately 105 kDa.

### Cell Signaling and Immunoblot Assays

Cellular reagents were purchased from various commercial vendors. Primary human umbilical vein endothelial cells (HUVECs) were obtained from Cell Applications Inc. and cultured according to the manufacture's recommendations. Primary human coronary artery smooth muscle cells were obtained from Invitrogen and were cultured according to the manufacture's recommendations. 293T cells were obtained from ATCC and were cultured according to the manufacture's recommendations. Mouse primary smooth muscle cells were obtained and cultured as described previously. Predesigned Silencer Select siRNA constructs targeting Pearl 1 and negative control siRNA were obtained from ThermoFisher. Transfections were performed using RNAiMAX or Lipofectamine 3000 transfection reagents according to the manufacturer's protocols. Cells were used for signaling assays 48 hours after transfection; efficient Pearl 1 knockdown or Pearl 1 overexpression was confirmed by immunoblot assays.

Prior to performing signaling assays, cells were trypsinized, centrifuged, suspended in basal media, and counted using an automated hemocytometer. Cells were further diluted in basal media to an assay-dependent concentration. Cells were then incubated with gentle agitation for 60 minutes to prevent cell attachment and reduce basal signaling. PP1, Dynasore, MK-2206, and Rapamycin were diluted in DMSO and used in assay concentrations of 10 μM, 100 μM, 10 μM, and respectively, for the final 20-30 minutes of incubation in basal media. An equal volume of DMSO was used as the negative control condition. 1 mL of the cell-culture was then seeded on 24-well tissue culture plates pre-coated with 15-30 μg/mL BSA or SVEP1 and washed 535 with Dulbecco's phosphate-buffered saline (DPBS). 1 μg Pearl 1 polyclonal antibody was added as a specific positive control. 20% growth media or 2% fetal bovine serum, labeled “Serum” in figures, was added as a non-specific positive control. The cells were centrifuged at 300G for 3 minutes with the exception of platelets, which were centrifuged at 500G for 5 minutes. The cells were then incubated at 37° C. for 8-45 minutes, depending on the cell type and pathway of interest. The cells were lysed in radioimmunoprecipitation buffer (RIPA) containing a cocktail of protease and phosphatase inhibitors and universal nuclease. Immunoblots were performed by standard techniques, as briefly follows. Protein content was determined using a bicinchoninic acid assay with BSA standards (#23225, Pierce BCA Protein Assay Kit). Cell lysates were then reduced with dithiothreitol (DTT) in lithium dodecyl sulfate (LDS) sample buffer (#NP0007, Invitrogen). Equal protein amounts were added to polyacrylamide gels (#4561086, BioRad) and electrophoresed prior to transferring to a nitrocellulose or polyvinylidene fluoride membrane (#1620260, BioRad). Membranes were blocked in 5% BSA/Tris-buffered saline (TBS) with 0.1% Tween 20 for 30 minutes. The indicated primary antibodies were incubated with the pre-blocked membranes overnight at 4° C. Membranes were washed with TBS with 0.1% Tween 20, probed with fluorescent secondary antibodies, and imaged. β-actin or β-tubulin served as a loading control.

For the protein array assays, HUVEC cell lysates were used with the C-Series Human and Mouse AKT Pathway Phosphorylation Array C1 (Raybiotech Inc.) according to the manufacturer's protocol. The signal intensity was normalized to the lysates from negative control BSA-coated wells. Plasma was pooled from two Svep1+/+ or Svep1−/− mice to constitute a single biological replicate for the mouse cytokine array C3 assay; two samples were derived from each sex of each mouse genotype for the assay.

### Adhesion Assay

Wells of a 96-well plate were pre-coated with 0, 1, 3, 10, 20, 30, and 50 μg/mL recombinant SVEP1 or 10 μg/mL plasma fibronectin (pFn, as a positive control) in DPBS. Subsequently, wells were blocked with 100 mg/mL BSA and washed with DPBS. HUVECs were collected and resuspended in basal media. 5,000 cells were added to each well. Cells were centrifuged and incubated at 37° C. for 10 minutes. The plate was gently centrifuged upside down to remove non-adhered cells. Adhered cells were fixed with 4% paraformaldehyde, stained with 4′,6-diamidino-2-phenylindole (DAPI), and counted by automated microscopy as done previously.

### Cell Imaging and Colocalization Analysis

HUVECs or si-RNA transfected hCASMCs were trypsinized and seeded on chamber slides precoated with 30 μg/mL SVEP1. Cells were incubated for one hour at 37° C., rinsed with DPBS, and fixed with 4% paraformaldehyde. Cells were washed, then blocked and permeabilized with 0.3% Triton X-100 and 5% chicken serum in TBS. Cells were incubated with Pearl 1 pAb and anti-pAKT antibody for two hours, washed with TBS +0.1% Tween 20, and incubated with secondary antibodies and phalloidin stain (for fActin), for 1 hour. Chambers not treated with the primary antibody were used as a negative control. Cells were washed and treated with Prolong Diamond Antifade with DAPI overnight at room temperature. The cells were imaged the following day using confocal microscopy. The fluorescent channel for fActin was used to identify cells and focus the microscope for imaging. Composite images were split into composite pseudocolors using Fiji. The fActin channel was used to identify lamellipodia (bundles of fActin on the periphery of cells) and control, non-lamellipodia cellular regions (see FIG. 51C for representative images). The Fiji plugin Coloc 2 was used to measure intensity-independent colocalization between the Pearl 1 and pAKT channels. Pearson's correlation coefficient was reported as a measure of colocalization between Pearl 1 and pAKT.

### Human Blood Collection and Platelet Isolation

Whole blood was collected by venipuncture into either heparin vacutainers (BD, Franklin Lakes) for whole blood experiments or acid-citrate-dextrose (ACD) vacutainers (BD, Franklin Lakes) for platelet isolation studies. For platelet isolation, samples were supplemented with apyrase (Sigma, St Louis) and prostaglandin E1 (Cayman Chemical, Ann Arbor), and platelet-rich plasma (PRP) was prepared by centrifugation of the ACD whole blood for 20 minutes at 200G. Platelets were isolated from the PRP by centrifugation for 10 minutes at 1000G and resuspended in modified Tyrode's buffer twice at desired platelet counts and kept at 37° C. until used. Isolated platelets were used within 2 hours of preparation.

### Static Adhesion Assays

Coverslips were pre-coated with either 15 μg/mL recombinant SVEP1, 100 μg/mL fibrinogen (as a positive control) or 1% BSA (as a negative control) in phosphate-buffered saline (PBS). Subsequently, coverslips were blocked with 1% BSA and washed with PBS. Human platelets were isolated from whole blood and re-suspended in Tyrode's buffer. 2×10⁷/mL basal or thrombin activated platelets were added to coverslips and incubated at 37° C. for 10, 20, or 30 minutes. After incubation, non-adherent platelets were removed and the coverslips were washed with PBS, fixed with 1.5% paraformaldehyde, permeabilized with 0.01% Triton-X and stained with TRITC phalloidin. Platelets were visualized using fluorescent microscopy. Images of the adhered platelets were captured using fluorescent microscopy and counted manually by a blinded observer.

### Blood Cell Counts and Flow Cytometry

Complete blood counts (CBC) and washed platelet counts were determined using a hematology analyzer Element HT5 (Heska, Loveland). For platelet aggregation experiments, whole blood platelet count was measured prior to SVEP1 addition and 15 minutes after the addition of 15 μg/mL SVEP1. For flow cytometry, diluted whole blood or isolated platelets were pre-incubated with their respective fluorescent antibodies for 15 minutes and fixed. For murine whole blood: CD41-VioBlue, CD61-PE (Mitlenyi Biotec, Bergisch Gladbach, Germany), CD42b-DL649, GPVI-FITC (Emfret, Eibelstadt, Germany) and for human whole blood and isolated platelets: CD41-FITC, CD61-APC, CD42b-PE (Biolegend, San Diego), GPVI-BV421 617 (BD, Franklin Lakes). Platelet surface receptor levels were quantified by flow cytometry on a 618 CytoFlex analyzer.

Quantification of Platelet Integrin aIIbβ3 Activation and P-Selectin Expression

Diluted human whole blood or isolated human platelets were treated with SVEP1 as described previously, pre-incubated with FITC-PAC-1 and P-selectin-PE antibodies (BD, Franklin Lakes), and stimulated with either ADP (10 μM) (Chronolog, Harverton); Thrombin receptor-activating peptide-6 (TRAP-6; 10 μM) (Tocris, Bristol, UK); Protease-activated receptor-4 activating peptide (PAR4-AP; 100 μM) (Abcam, Cambridge, UK) or Thrombin (0.1 U/mL) (Chronolog, Harverton) for 15 minutes. Samples were immediately fixed and run using a CytoFlex analyzer (Beckman Coulter, Pasadena) and analysis was performed with Kaluza software (Beckman Coulter, Pasadena).

For murine whole blood flow cytometry, whole blood was collected from the retro-orbital plexus using heparinized capillary tubes. Diluted whole blood was pre-incubated with fluorescently conjugated JON/A-FITC and CD62P-PE antibodies (Emfret, Eibelstadt, Germany) and stimulated with either ADP (10 μM), PAR4-AP (100 μM) or Thrombin (0.1 U/mL) for 15 minutes. After incubation, samples were immediately fixed and read on a CytoFlex analyzer.

### Mice

The generation and validation of an inducible Svep1−/−636 allele and mouse model were described previously. In brief, mice were generated by KOMP (Knockout Mouse Project) and crossed with mice expressing the flippase FLP recombinase under the control of the promoter of the human actin beta gene to generate Svep1flx/flx mice. These mice were crossed with Rosa26-CreERT2 (no. 008463, the Jackson Laboratory) mice to generate Svep1flx/+Rosa26-CreERT2 mice. Male and female Svep1flx/+Rosa26-CreER T2 were crossed to generate experimental Svep1flx/flxRosa26-CreERT2 (Svep1−/−) and Svep1+/+Rosa26-CreERT2 (Svep1+/+) littermate control mice. To activate Cre-recombinase, mice were injected intraperitoneally with 2.5 mg of tamoxifen (no. T5648, Sigma-Aldrich) in 0.1 mL of peanut oil (no. P2144, Sigma-Aldrich) for 5 consecutive days starting at 6 weeks of age. Tamoxifen treatment was performed with all experimental and control mice in an identical manner. Given the cardiometabolic and age-related disease associations of SVEP1 in humans, aged Svep1−/− and control Svep1+/+ mice fed a western diet comprised of 21% fat by weight (42% kcal from fat) and 0.2% cholesterol (#TD.88137, Envigo Teklad) were used beginning at 8 weeks of age. This diet was referred to as “HFD” throughout the text. The metabolic phenotyping of these mice occurred between 8-9 months of age, the hematological phenotyping occurred between 10-12 months of age, and the vascular phenotyping occurred between 12-13 months of age.

The mice referred to in this text as “Pear1−/−” are the Pear1tmla(KOMP)Wtsi mice generated by KOMP (generously provided by Dr. Bruce Carter, Vanderbilt University). The “Pear1+/+” control mice are age and background-matched C57BL/6NCrl mice (Charles River Laboratories) and were acclimated in the same facility as the Pear1−/− mice for at least one week prior to the experiments. The Pear1−/− and Pear1+/+ mice were fed a standard chow diet and were assessed at 6 weeks of age. All mice were housed in the Washington University School of Medicine animal facility and maintained on a 12-hour light/12-hour dark cycle with a room temperature of 22±1° C.

### Arterial Blood Pressure Measurement

Central arterial blood pressure and heart rate were measured under inhaled 1.5% isoflurane anesthesia and while mice were maintained at 37° C. using a heating pad and rectal thermometer, as done previously. Briefly, a midline incision was performed in the neck region; the thymus, muscle, and connective tissue were dissected away to isolate the right common carotid artery. After tying it distally and clamping it proximally, an incision was made in the right common carotid artery through which a Millar pressure transducer (model SPR-1000, Houston, Tex.) was introduced, the clamp was removed, and the transducer advanced to the ascending aorta. Once instrumentation was complete, arterial blood pressure (systolic, diastolic, and mean) and heart rate were recorded via the PowerLab® data acquisition system (ADlnstruments, Colorado Springs, Colo.). The average of a 3-minute period of stable recording was reported. Data were analyzed using LabChart® 8 for 673 MAC software (ADInstruments).

To assess the blood pressure response to vasoactive agents, after baseline blood pressure measurement, dissection was performed to visualize the left internal jugular (IJ) vein as done previously. Once identified, a small incision was made and PE-10 tubing was introduced and kept in place with a 6-0 silk suture. While measuring arterial blood pressure, 50 μL normal saline (NS) was injected via the IJ line as a bolus injection (1-2 seconds). After 2-3 minutes, baseline blood pressure was noted and increasing concentrations of either phenylephrine, angiotensin II, acetylcholine, or sodium nitroprusside (Sigma, St. Louis, Mo.) each in a ˜10 μL volume were injected in the IJ line and flushed with 40 μL NS. After each drug, when blood pressure returned to baseline (2-3 minutes), the line was washed with 50 μL NS for 3 minutes. The maximal change in blood pressure after each dose was reported. Mice were euthanized at the completion of the experiment.

### Pressure Myography

Following blood pressure measurement and euthanasia, ascending aorta and left common carotid artery were excised and placed in physiologic saline solution (PSS) composed of 130 mM NaCl, 4.7 mM KCl, 1.6 mM CaCl₂, 1.18 mM MgSO₄-7H₂O, 1.17 mM KH₂PO₄, 14.8 mM NaHCO₃, 5.5 mM dextrose, and 0.026 mM Ethylenediaminetetraacetic acid (EDTA, pH 7.4) overnight at 4° C. Vessels were cleaned of surrounding fat, mounted on a pressure arteriograph (Danish Myo Technology), and maintained in PSS at 37° C. Vessels were visualized with an inverted microscope connected to a CCD camera and a computerized system, which allows continuous recording of vessel diameter. Intravascular pressure was increased from 0 to 175 mmHg by 25-mmHg increments, the vessel outer diameter was recorded at each step (12 seconds per step). The average of three measurements at each pressure was reported.

### Metabolic Phenotyping

Male Svep1+/+ and Svep1−/− mice were weighed and fasted for 5 hours prior to the metabolic challenge. For insulin tolerance tests (ITT), Humulin R (100 units/mL) was diluted 1:1000 in sterile PBS and injected intraperitoneally in mice at a dose of 0.75 units/kg. For glucose tolerance tests (GTT), a 20% glucose solution was prepared in sterile PBS and injected intraperitoneally at a final dose of 2g/kg. Tail vein glucose measurements were collected at 15 30-minute intervals using a glucometer. Mouse lean, fat, and total water mass were determined in male Svep1+/+ and Svep1−/− mice fed HFD using EchoMRI (EchoMRI LLC). The EchoMRI was calibrated with canola oil. Measurements were gathered in duplicate for each mouse and averaged prior to analysis. Indirect calorimetry measurements were collected using the PhenoMaster System (TSE Systems) in collaboration with the Washington University Diabetes Research Center Diabetes Models Phenotyping Core. Male Svep1+/+ and Svep1−/− mice fed HFD were placed in individual chambers and acclimated for several hours prior to data collection. Mice were fed HFD throughout the data collection. The measurements occurred at room temperature during standard 12-hour light/12-hour dark cycles.

### Proteomic Pulldown Assays

Affinity-based proteomics: Murine VSMCs were grown to confluence in serum-containing media and changed to serum-free media to generate enriched media. Recombinant, Myc-tagged SVEP1 was added to the media after two days of enrichment and incubated for 1 hour. An aliquot was removed after incubation as the “input” fraction. Media was then added to a slurry of Pierce Anti-c-Myc Magnetic Beads+0.05% Tween 20 and incubated for 30 minutes while rocking at 4° C. The beads were then washed twice with Ca²⁺ and Mg²⁺-containing PBS (D8662, Sigma) +0.05% Tween 20 and twice with Ca²⁺ and Mg²⁺-containing PBS before a final resuspension in PBS. An aliquot of the beads was reserved as the pulldown fraction for validation. Proximity-based proteomics: recombinant SVEP1-Myc or SVEP1-mTID was added to enriched VSMC media with 500 μM exogenous biotin and 1 mM adenosine triphosphate (ATP). The samples were incubated for 4 hours prior to dialysis. Protease arrest (G-Biosciences) was added and excess biotin was dialyzed using a 10 kDa molecular weight cut-off (MWKO) Slide-A-Lyzer™ dialysis Cassette (ThermoFisher Scientific) in buffered saline+1 mM EDTA. The samples were then transferred to 10 kDa MWKO Vivasin column (Sartorius Stedim Biotech), centrifuged, resuspended in RIPA buffer, centrifuged, and added to pre-washed Pierce Streptavidin Magnetic Beads. The samples and beads were incubated for 1 hour at room temperature or overnight at 4° C. and then washed with the following solutions: RIPA buffer, 1M 732 KCl, 0.1M Na₂CO₃, 2M urea in 10 mM Tris HCl (pH=8.0), and PBS. The beads were then resuspended in PBS for peptide preparation.

### Peptide Preparation

The peptides were prepared using a previously described method for on-bead tryptic digestion. The beads were washed four times with 1 mL of 50 mM ammonium bicarbonate buffer (pH=8.0) (ABC). The washed beads were resuspended in 40 μL of ABC buffer containing 8M urea. The protein disulfide bonds were reduced using 2 μL of 0.5M TCEP and incubation for 60 minutes at 30° C. The reduced proteins were alkylated using 4 μL of a 0.5M solution of iodoacetamide with incubation for 30 minutes at RT in the dark. The urea was diluted to a concentration of 1.5M by adding 167 μL of 50 mM ABC buffer. After the addition of LysC (1mAU), the samples were incubated for 2 hours at 30° C. in a Thermomixer with gyration at 750 rpm. Trypsin (1 μg) was added and the samples were incubated overnight at 30° C. in the Thermomixer gyrating at 750 rpm. The peptides were transferred to a 1.5 mL tube, the beads were washed with 50 μL of ABC buffer and the transfer and wash volumes were combined. Any residual detergent was removed by ethyl acetate extraction. The peptide samples were acidified with TFA to a final concentration of 1% (vol/vol) TFA (pH <2.0). The pH was checked with pH paper. The peptides were desalted using two micro-tips (porous graphite carbon, BIOMETNT3CAR) (Glygen) on a Beckman robot (Biomek NX), as previously described. The peptides were eluted with 60% (vol/vol) MeCN in 0.1% (vol/vol) TFA. After adding TFA to a final concentration of 5%, the peptides were dried in a Speed-Vac (Thermo Scientific, Model No. Savant DNA 120 concentrator). The peptides were dissolved in 20 μL of 1% (vol/vol) MeCN in water. An aliquot (10%) was removed for quantification using the Pierce Quantitative Fluorometric Peptide Assay kit (Thermo Scientific, Cat. No. 23290). The remainder was transferred to autosampler vials (Sun-Sri, Cat. No. 200046), dried, and stored at −80° C. Peptides were also prepared after the release of proteins from antibody beads. The beads were washed with 1 mL of 50 mM cold phosphate-buffered saline (pH 8.0) (PBS) followed by elution with 30 μL of SDS buffer (4% (wt/vol), 100 mM Tris-HCl pH 8.0). The protein disulfide bonds were reduced using 100 mM DTT with heating to 95° C. for 10 minutes. Peptides were prepared as previously described using a modification of the filter-aided sample preparation method (FASP). The samples were mixed with 200 of 100 mM Tris-HCL buffer, pH 8.5 containing 8M urea (UA buffer). The samples were transferred to the top chamber of a 30,000 MWCO cutoff filtration unit (Millipore, part# MRCF0R030) and spun in a microcentrifuge at 14,000G for 10 minutes. An additional 200 μL of UA buffer was added and the filter unit was spun at 14,000G for 15 to 20 minutes. The cysteine residues were alkylated using 100 μL of 50 mM Iodoacetamide (Pierce, Ref. No. A39271) in UA buffer. Iodoacetamide in UA buffer was added to the top chamber of the filtration unit. The samples were gyrated at 550 rpm for 30 minutes in the dark at RT using a Thermomixer (Eppendorf). The filter was spun at 14,000rcf for 15 minutes and the flow through was discarded. Unreacted iodoacetamide was washed through the filter with two sequential additions of 200 μL of 100 mM Tris-HCl buffer, pH 8.5 containing 8M urea, and centrifugation at 14,000 rcf for 15 to 20 minutes after each buffer addition. The flow-through was discarded after each buffer exchange-centrifugation cycle. The urea buffer was exchanged with digestion buffer (DB), 50 mM ammonium bicarbonate buffer, pH 8. Two sequential additions of DB (200 μL) with centrifugation after each addition to the top chamber were performed. The top filter units were transferred to a new collection tube and 100 μL DB containing 1mAU of LysC (Wako Chemicals, cat. no. 129-02541) was added, and samples were incubated at 37° C. for 2 hours. Trypsin (1 μg) (Promega, Cat. No. V5113) was added and samples were incubated overnight at 37° C. The filters were spun at 14,000 rcf for 15 minutes to recover the peptides in the lower chamber. The filter was washed with 50 μL of 100 mM ABC buffer and the wash was combined with the peptides. Residual detergent was removed by ethyl acetate extraction. After extraction, the peptides were dried in a Speedvac concentrator (Thermo Scientific, Savant DNA 120 Speedvac Concentrator) for 15 minutes. The dried peptides were dissolved in 1% (vol/vol) TFA and desalted using two micro-tips (porous graphite carbon, BIOMEKNT3CAR) (Glygen) on a Beckman robot (Biomek NX), as previously described. The peptides were eluted with 60 μL of 60% (vol/vol) MeCN in 0.1% (vol/vol) TFA and dried in a Speed-Vac (Thermo Scientific, Model No. Savant DNA 120 concentrator) after adding TFA to 5% (vol/vol). The peptides were dissolved in 20 μL of 1% (vol/vol) MeCNA in water. An aliquot (10%) was removed for quantification using the Pierce Quantitative Fluorometric Peptide Assay kit (Thermo Scientific, Cat. No. 23290). The remaining peptides were transferred to autosampler vials (Sun-Sri, Cat. No. 200046), dried, and stored at −80° C.

### UPLC-TimsTOF Mass Spectrometry

The peptides were analyzed using a nano-Elute chromatograph coupled online to a hybrid trapped ion mobility-quadrupole time of flight mass spectrometer (timsTOF Pro, Bruker Daltonics, Bremen Germany) with a modified nano-electrospray source (CaptiveSpray, Bruker Daltonics). The mass spectrometer was operated in PASEF mode. The samples in 1% (vol/vol) aqueous FA were loaded (2 μL) onto a 75 μm i.d.×25 cm Aurora Series column with CSI emitter (Ionopticks) on a Bruker nano-ELUTE (Bruker Daltonics). The column temperature was set to 50° C. The column was equilibrated using constant pressure (800 bar) with 8 column volumes of solvent A (0.1% (vol/vol) aqueous FA)). Sample loading was performed at constant pressure (800 bar) at a volume of 1 x sample pick-up volume plus 2 μL. The peptides were eluted using the one column separation mode with a flow rate of 400 nL/min and using solvents A and B (0.1% (vol/vol) FA/MeCN): solvent A containing 2% B increased to 15% B over 60 minutes, to 25% B over 30 minutes, to 35% B over 10 min, to 80% B over 10 minutes and constant 80% B for 10 minutes. The MS1 and MS2 spectra were recorded from m/z 100 to 1700. Suitable precursor ions for PASEF-MS/MS were selected in real-time from TIMS-MS survey scans by a PASEF scheduling algorithm. A polygon filter was applied to the m/z and ion mobility plane to select features most likely representing peptide precursors rather than singly charged background ions. The quadrupole isolation width was set to 2Th for m/z <700 and 3 Th for m/z >700, and the collision energy was ramped stepwise as a function of increasing ion mobility: 52 eV for 0-19% of the ramp time; 47 eV from 19-38%; 42 eV from 38-57%; 37 eV from 57-76%; and 32 eV for the remainder. The TIMS elution voltage was calibrated linearly using the Agilent ESI-L Tuning Mix (m/z 622, 922, 1222).

### UPLC-Orbitrap Mass Spectrometry

The samples in formic acid (1%) were loaded (2.5 μL) onto a 75 μm i.d.×50 cm Acclaim PepMap 100 C18 RSLC column (Thermo-Fisher Scientific) on an EASY nanoLC (Thermo Fisher Scientific) at a constant pressure of 700 bar at 100% A (0.1%FA). Prior to sample loading, the column was equilibrated to 100% A for a total of 11 μL at 700 bar pressure. Peptide chromatography was initiated with mobile phase A (1% FA) containing 2% B (100%ACN, 1%FA) for 5 minutes, then increased to 20% B over 100 minutes, to 32% B over 20 minutes, to 95% B over 1 minute and held at 95% B for 19 minutes, with a flow rate of 300 nL/minute. For a lower flow rate (250 nL/minute), 95% B was held for 29 minutes. The data were acquired in data-dependent acquisition (DDA) mode. The full-scan mass spectra were acquired with the Orbitrap mass analyzer with a scan range of m/z=325 to 1500 (350 to 1500) and a mass resolving power set to 70,000. Ten data-dependent high-energy collisional dissociations were performed with a mass resolving power set to 17,500, a fixed lower value of m/z 100, an isolation width of 2 Da, and a normalized collision energy setting of 27. The maximum injection time was 60 ms for parent-ion analysis and product-ion analysis. The target ions that were selected for MS/MS were dynamically excluded for 20 seconds. The automatic gain control (AGC) was set at a target value of 3e6 ions for full MS scans and le5 ions for MS2. Peptide ions with charge states of one or >8 were excluded for HCD acquisition.

### Identification of Proteins

The data from the tims-TOF Data mass spectrometer were converted to peak lists using DataAnalysis (version 5.2, Bruker Daltonics). The MS2 spectra with charges +2, +3, and +4 were analyzed using Mascot software (Matrix Science, London, UK; version 2.5.1). Mascot was set up to search against a UniProt (ver October 2013) database of mouse proteins (43,296 entries), assuming the digestion enzyme was trypsin with a maximum of 2 missed cleavages allowed. The searches were performed with a fragment ion mass tolerance of 50 ppm and a parent ion tolerance of 50 ppm. Carbamidomethylation of cysteine was specified in Mascot as a fixed modification. Deamidation of asparagine, deamidation of glutamine, acetylation of protein N-845 terminus, and oxidation of methionine were specified as variable modifications. Peptides and proteins were filtered at a 1% false-discovery rate (FDR) by searching against a reversed protein sequence database. MS raw data were converted to peak lists using Proteome Discoverer (version 2.1.0.81, Thermo-Fischer Scientific). MS/MS spectra with charges greater than or equal to two were analyzed using Mascot search engine (Matrix Science, London, UK; version 2.7.0). Mascot was set up to search against a UniProt database of the mouse (version October 2013, 43,296 entries), assuming the digestion enzyme was trypsin with a maximum of 4 missed cleavages allowed. The searches were performed with a fragment ion mass tolerance of 0.02 Da and a parent ion tolerance of 20 ppm. Carbamidomethylation of cysteine was specified in Mascot as a fixed modification. Deamidation of asparagine, formation of pyro-glutamic acid from N-terminal glutamine, acetylation of protein N-terminus, and oxidation of methionine were specified as variable modifications. Peptides and proteins were filtered at a 1% false-discovery rate (FDR) by searching against a reversed protein sequence database.

### Mass Spectrometry Analysis

A cumulative binomial distribution was used to determine which proteins were enriched at a threshold of P<0.10 in the samples containing SVEP1 compared to negative control samples. The probability of success on a single trial was set to the null hypothesis of 0.5. Proteins were considered “Hits” if they achieved a reproducibility criterion of P<0.10 in three controlled experiments and a fourth experiment that lacked an experimental negative control. A meta-analysis was performed on the three independent, controlled experiments using Fisher's method.

### Gene Cloning

Full-length Pearl 1: Human Pearl 1 cDNA was obtained from a pDONR221 plasmid (HSCD00863115, DNASU plasmid repository) by PCR and cloned into a modified pCMV6 plasmid (OriGene, Rockville, Md.) with a Myc and poly-histidine C-terminal tag. The empty pCMV6 plasmid was used as the empty vector control in experiments. SVEP1 miniTurbo fusion protein (SVEP1-mTID): MiniTurbo cDNA (the promiscuous biotin ligase) was amplified by PCR from the V5-miniTurbo-NES-pCDNA3 plasmid (a gift from Alice Ting, Addgene, plasmid 107170) and cloned downstream of the murine Svep1sequence and upstream of the Myc and poly-histidine tag in the pCMV6 plasmid. Pearl 1ECD-Bio-His: The plasmid used for the Pearl 1 ecto-domain expression was pTT3-Pearl 1-bio-His (a gift from Gavin Wright, Addgene plasmid 51860). Svep1: cloning of mouse Svep1cDNA, protein expression, and purification was described in detail previously. Briefly, all proteins were expressed in 293F cells (Invitrogen) and grown in FreeStyle expression media. Plasmids were transfected with 3 μg/mL of vector DNA plus 9 μg/mL Polyethylenimine (PEI) (25 kDa linear PEI, Polysciences, Inc.) at a cell density of 2.5×10⁶ cells/mL. For the Pearl 1 ecto-domain biotinylation, 0.3 μg/μL of secreted BirA-8his plasmid (a gift from Gavin Wright, Addgene plasmid 32408) was co-transfected and supplemented with 0.1 mM biotin. Proteins were purified in an NGC chromatographic system (BioRad Lab) with 5 mL Nuvia IMAC resin (BioRad Lab) and polished using a Superose 6 increase 10/300 column (GE LifeSciences) with PBS as a carrier buffer.

### Results

### Plasma SVEP1 Concentration is Altered by Pearl 1

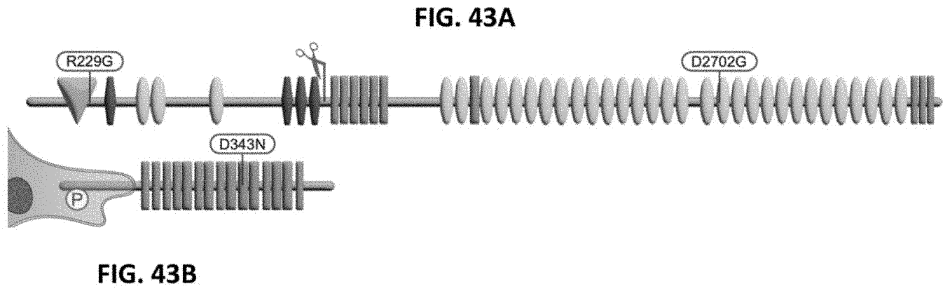

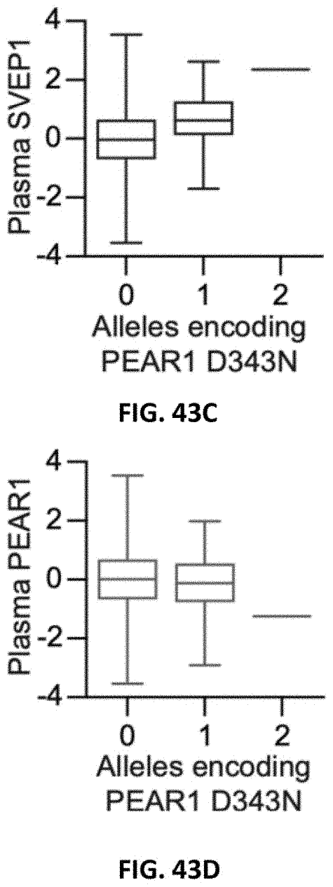

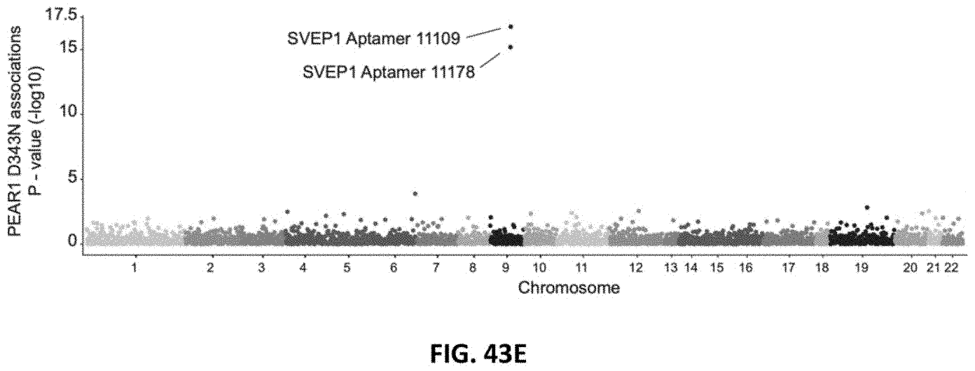

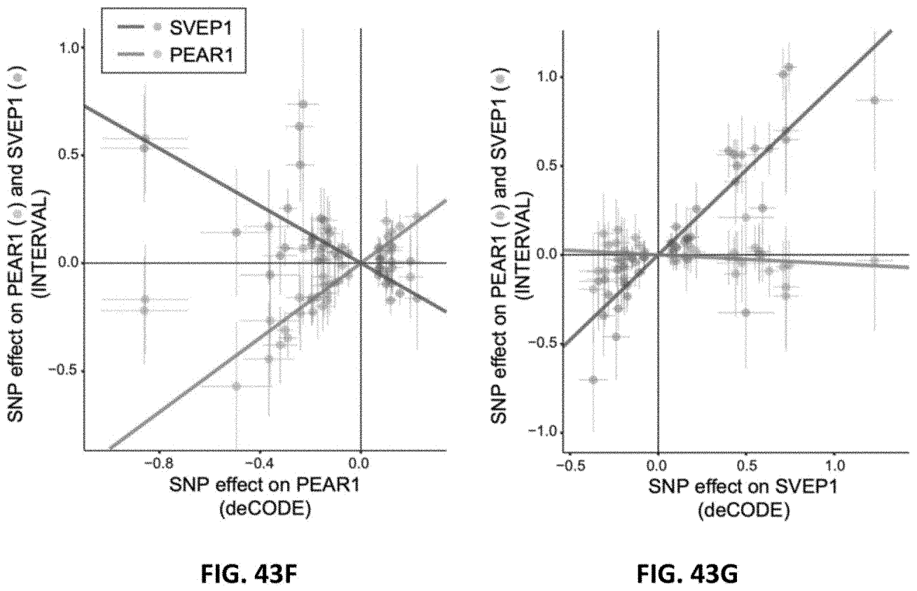

First, candidate SVEP1 interactions using human genomics and aptamer-based plasma proteomics from the INTERVAL study of healthy volunteers were attempted to be identified. Genetic variation within the locus containing SVEP1 on chromosome 9 influences plasma levels of SVEP1; these cis-protein quantitative trait loci (cis-pQTL) have been described previously. Strikingly, a genetic variation on chromosome 1 (a trans-protein quantitative trait locus, or trans-pQTL) also associates with altered plasma SVEP1 concentrations at a genome-wide level of significance. The trans pQTL variant most strongly associated with altered plasma SVEP1 concentration (r5145662369) is an intronic single nucleotide polymorphism (SNP) within the locus containing Pearl 1. rs145662369 is not associated with altered expression of Pearl 1 in the Genotype-Tissue Expression database (GTEx); however, it is in perfect linkage disequilibrium (r2=1) with rs147639000. rs147639000 is a missense polymorphism within an EGF-like domain of Pearl 1′ s ectodomain (FIG. 43B, p.D343N), and the minor allele (Asparagine at 343) is significantly associated with increased levels of plasma SVEP1 (P=6.5×10⁻¹⁶, FIG. 43C, 49A and 49B). Plasma Pearl 1 concentration is minimally altered in individuals harboring the Pearl 1 D343N variant (P=0.03, FIG. 43D). A proteome-wide association analysis of Pearl 1 D343N demonstrates that its impact on plasma protein is specific to SVEP1 among proteins measured by SomaScan (FIG. 43E).

Given the impact of the Pearl 1 D343N variant on plasma SVEP1 concentration and expression of Pearl 1 on vascular endothelial cells, it was hypothesized that Pearl 1 binds and sequesters circulating SVEP1 from human plasma. To test this hypothesis, it was asked whether genetic variation within the Pearl 1 locus that influences plasma Pearl 1 concentrations also impacts plasma SVEP1 concentrations. To avoid potential sources of confounding in a one-sample MR, a genetic instrument for plasma Pearl 1 and SVEP1 levels was generated using the recently published data from deCODE, a dataset of plasma protein levels measured by SomaScan in 35,559 Icelanders. Using the instruments generated from deCODE, it was asked if the genetically determined plasma levels of these proteins were associated with plasma protein levels from INTERVAL. Both instruments were able to accurately predict plasma concentrations of their respective proteins (Pearl 1 P=2.4×10⁻³⁷, FIG. 43F; SVEP1 P=2.5×10⁻⁵⁴, FIG. 43G), supporting the approach. Genetically encoded changes in plasma Pearl 1 concentration were inversely related to plasma SVEP1 (P=3.5×10⁻²⁰, FIG. 43F). As expected, plasma concentrations of Pearl 1 were minimally impacted by genetically encoded changes in plasma SVEP1 levels (P=0.002, FIG. 43G).

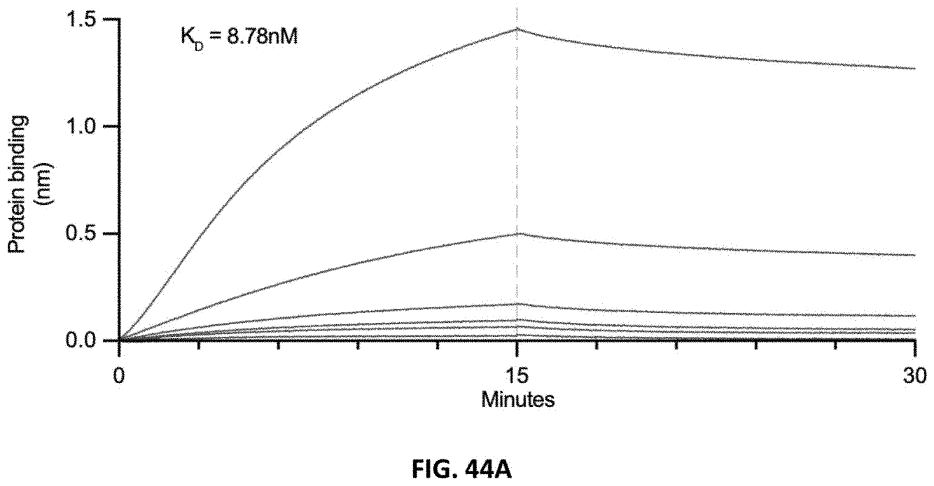

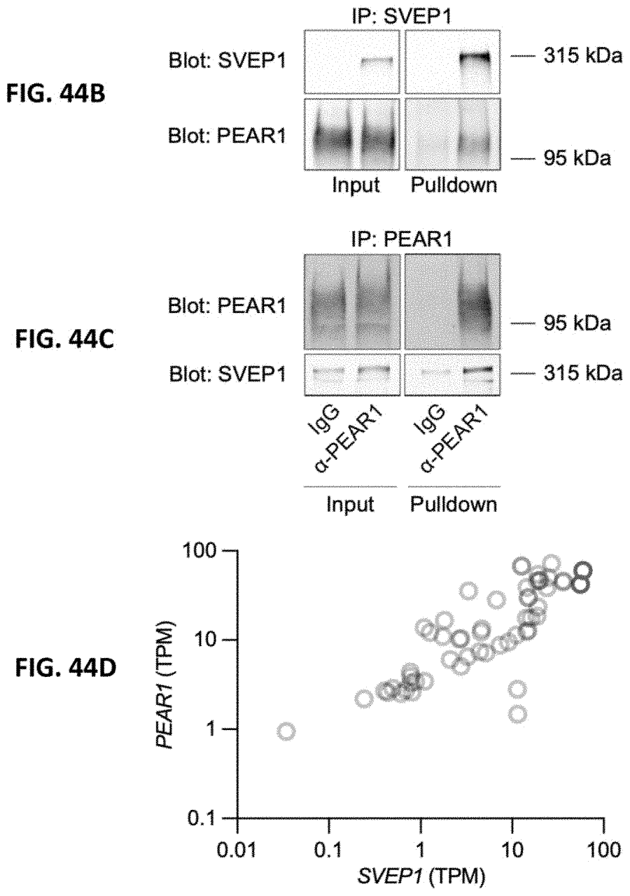

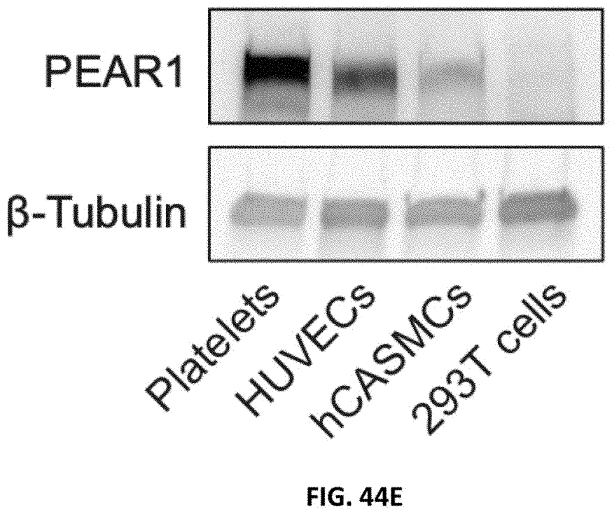

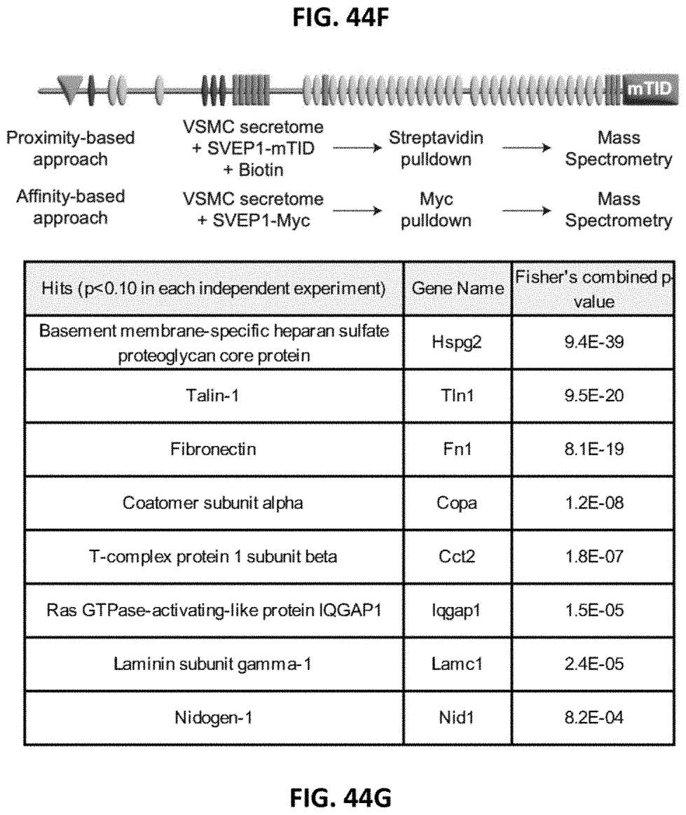

It was then asked whether SVEP1 and Pearl 1 physically interact using molecular assays. Biolayer interferometry (BLI) was used to perform a label-free, protein-binding analysis between recombinant Pearl 1 and SVEP1. A global fitting of a kinetic curve from an SVEP1 dilution series yielded a calculated dissociation constant (KD) of 8.78±0.03 nM (FIG. 44A). The extracellular domain (ECD) of Pearl 1 (Pearl 1ECD) also co-immunoprecipitates with recombinant, Myc-tagged SVEP1 in pulldown assays (FIG. 44B). An alternative Pearl 1ECD construct containing a biotin tag also coimmunoprecipitates with SVEP1 (FIG. 50A). Reciprocally, SVEP1 coimmunoprecipitates with the Pearl 1ECD (FIG. 44C). These molecular assays and MR analyses suggest SVEP1 and Pearl 1 physically interact with an affinity similar to tyrosine kinases and their ligands.

### SVEP1 and Pearl 1 are Co-Expressed in Human Tissues

SVEP1 circulates in human plasma, but the protein is thought to primarily reside within the ECM of the tissues where it is produced, similar to other ECM proteins. Pearl 1 also acts locally as a receptor that signals intracellularly. Therefore tissues that may harbor a biologically relevant interaction between SVEP1 and Pearl 1 were attempted to be identified by determining which tissues co-express their transcripts. The expression of SVEP1 and Pearl 1 is highly correlated among tissues in GTEx (FIG. 44D) and several tissues express high levels of both genes. For example, arterial and adipose tissues (orange and purple, respectively) express SVEP1 and Pearl 1 and are particularly relevant to cardiometabolic disease. Bone marrow is not among the tissues analyzed in GTEx; however, other sources of expression data indicate high expression of SVEP1 and Pearl 1 within this tissue. Single-cell RNA analysis of coronary arteries, the site of atherosclerosis that can lead to myocardial infarction, reveals that SVEP1 is expressed predominantly by fibroblasts (FIG. 50B), although studies in mice suggest that VSMCs may also express Svep1under pathological conditions. Pearl 1 is expressed by a variety of disease-relevant cell types within coronary arteries, including fibroblasts, smooth muscle cells, and endothelial cells (FIG. 50B). To assess protein expression of Pearl 1, platelet lysates from freshly isolated human platelets and cultured primary human umbilical vein endothelial cells (HUVECs), primary human coronary artery smooth muscle cells (hCASMCs), and 293T cells were collected. Immunoblot assays for Pearl 1 revealed that platelets, HUVECs, and hCASMCs express the protein. In contrast, 293T cells do not express an appreciable amount of Pearl 1 (FIG. 44E).

Receptors on the surface of cells interact with the ECM and influence cell behavior. The ECM is heterogenous and differences in its composition result in disparate effects on cells. Although SVEP1 is a canonical component of the ECM, antibodies that reliably recognize SVEP1 in situ have not been successfully developed; therefore, little is known about how and where SVEP1 may integrate into the ECM or which cell types it may influence. A combination of affinity and proximity-based experimental approaches were used to address this question. The bait proteins for these experiments included recombinant SVEP1 fused to a Myc-tag or mini-Turbo ID (mTID), a promiscuous biotin ligase. The prey proteins were derived from enriched media from murine VSMCs (FIG. 44F), a rich source of diverse ECM proteins. Two independent experiments were performed using each approach and a reproducibility criterion of P<0.10 for enrichment was applied across all experimental data. In addition to SVEP1, a total of 8 proteins fulfilled this criterion (FIG. 44G), including Basement membrane-specific heparan sulfate proteoglycan core protein (HSPG2, also known as Perlecan), Fibronectin, Laminin subunit gamma-1, and Nidogen-1. Pulldown of Fibronectin by SVEP1 was confirmed using coimmunoprecipitation assays (FIG. 50C). Together, these proteins comprise the major non-collagen basement membrane components. This suggests that SVEP1 may be integrated with the basement membrane where it could interact with numerous Pearl 1-expressing cells.

SVEP1 Signals through Pearl 1 to Activate AKT Signaling

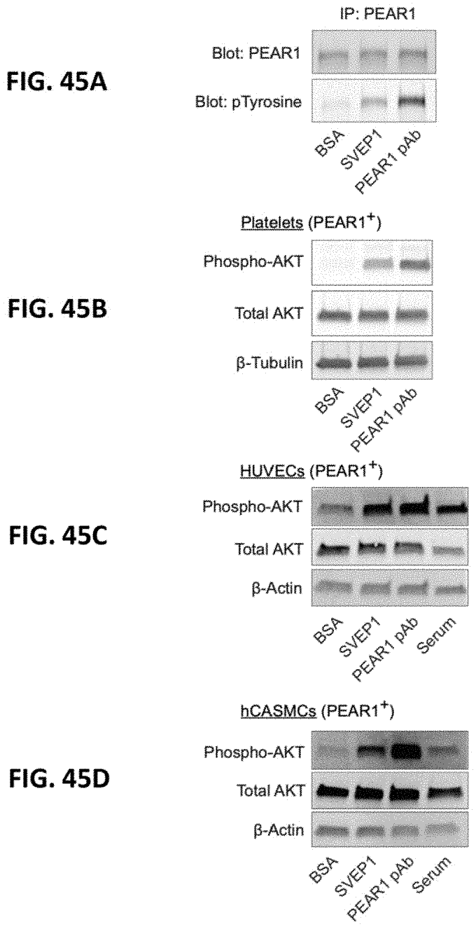

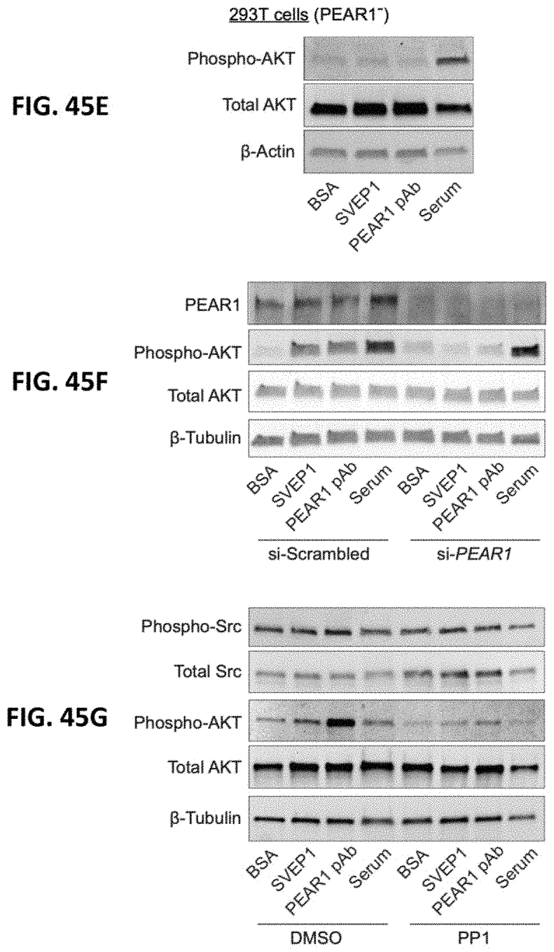

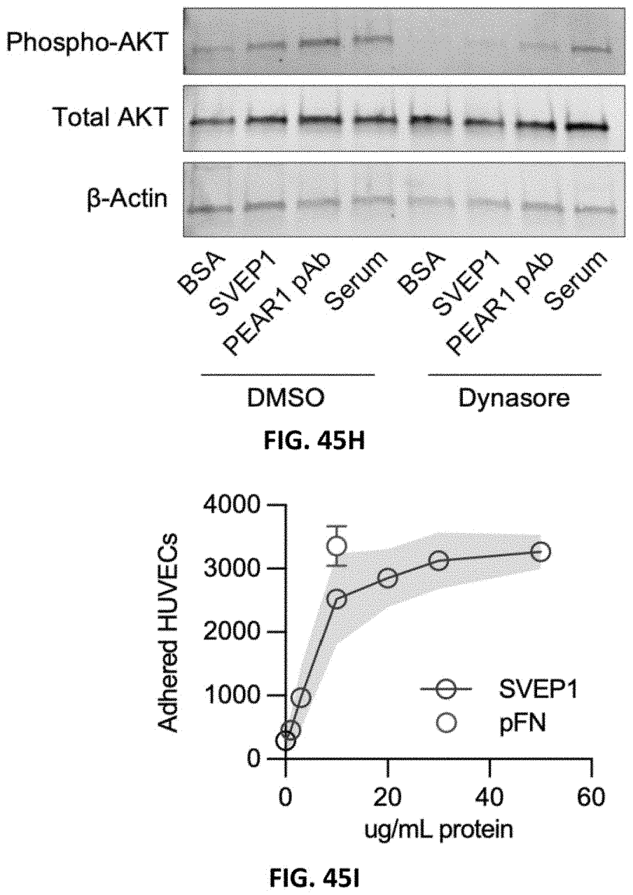

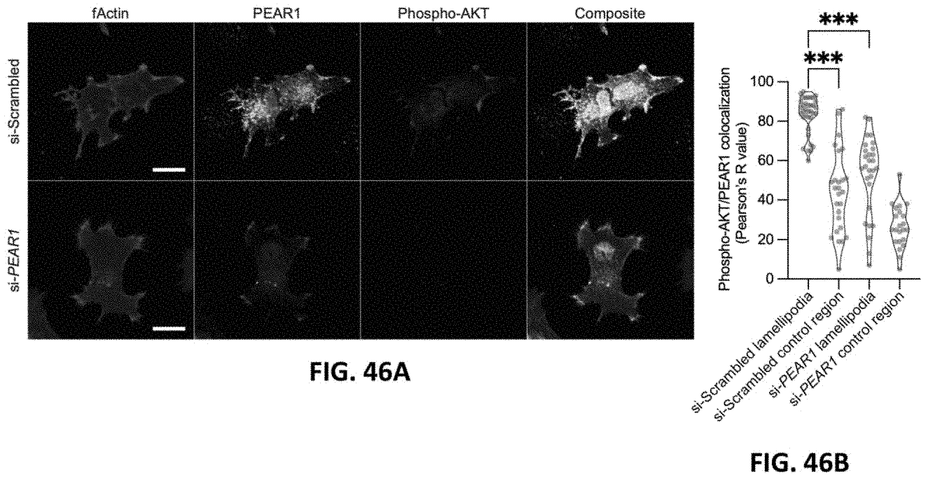

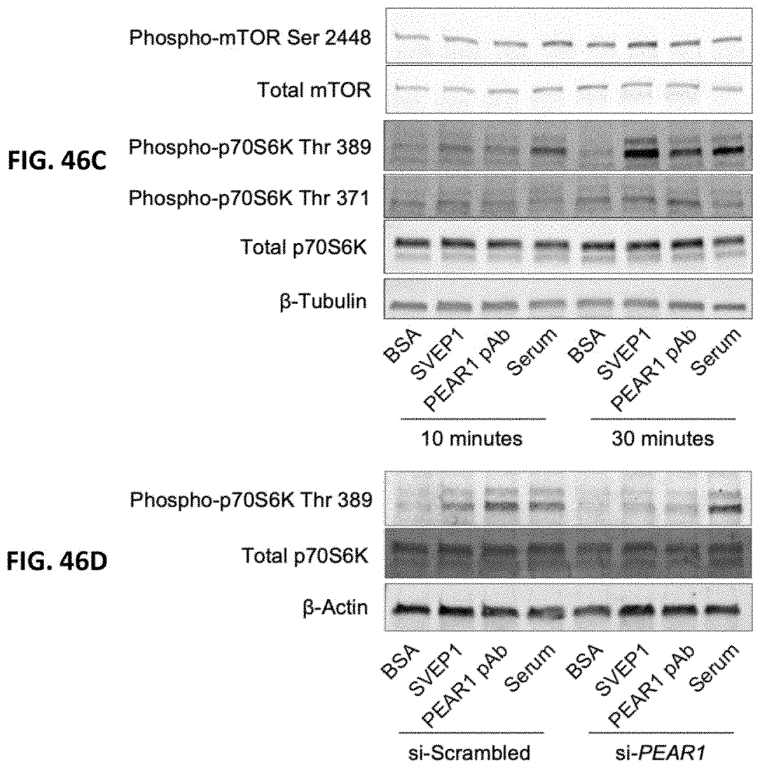

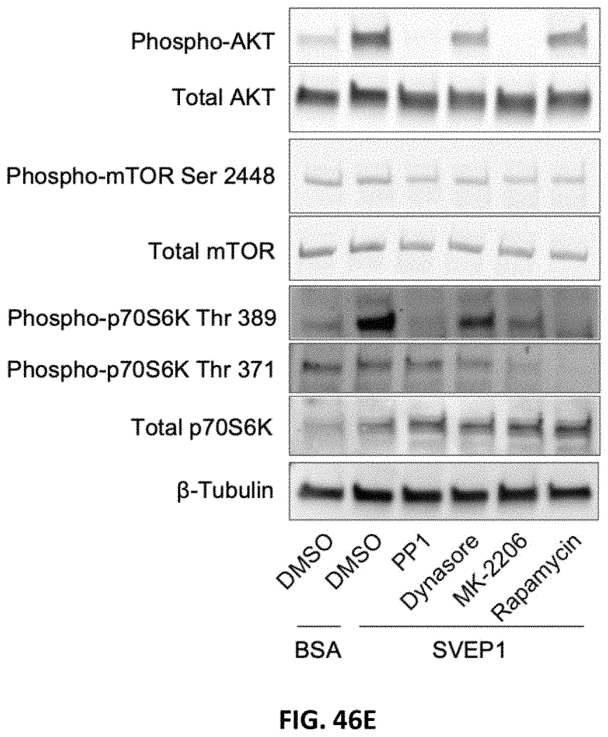

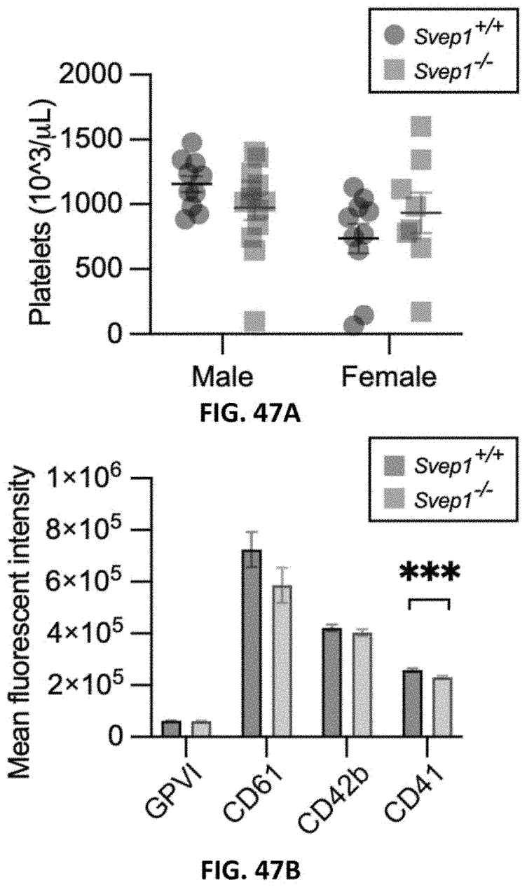

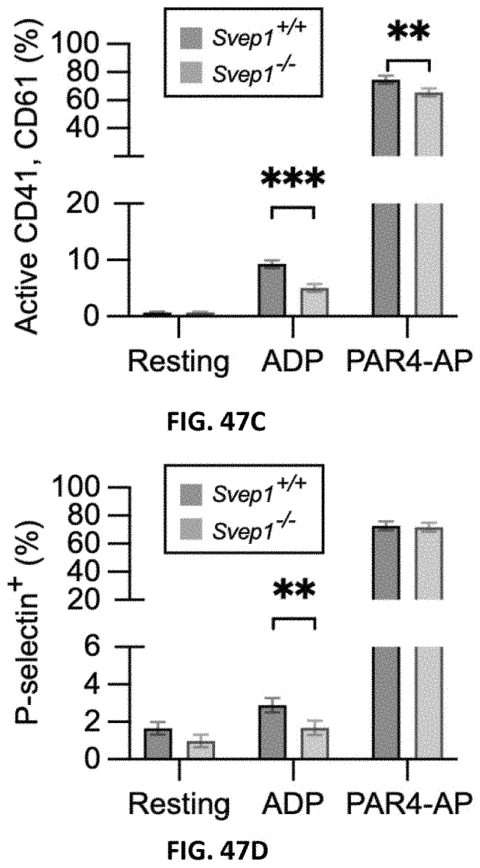

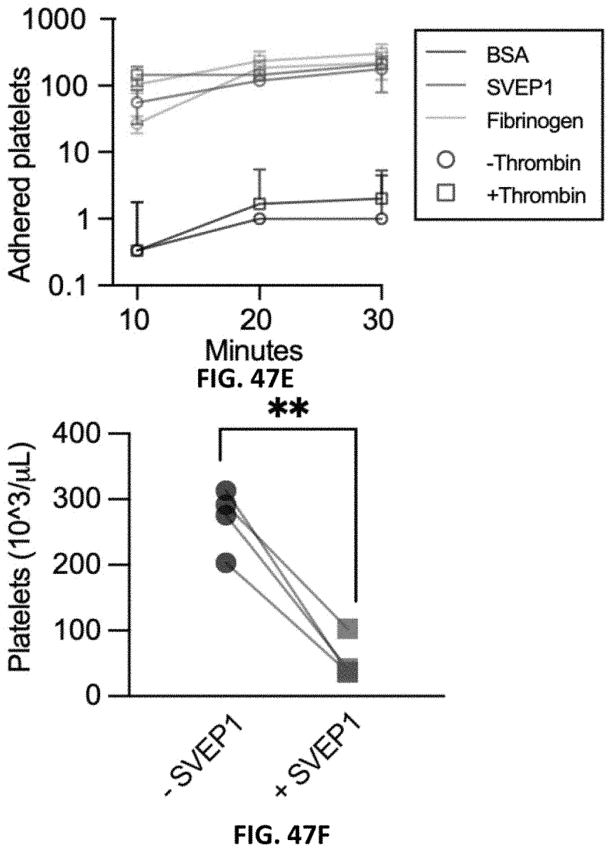

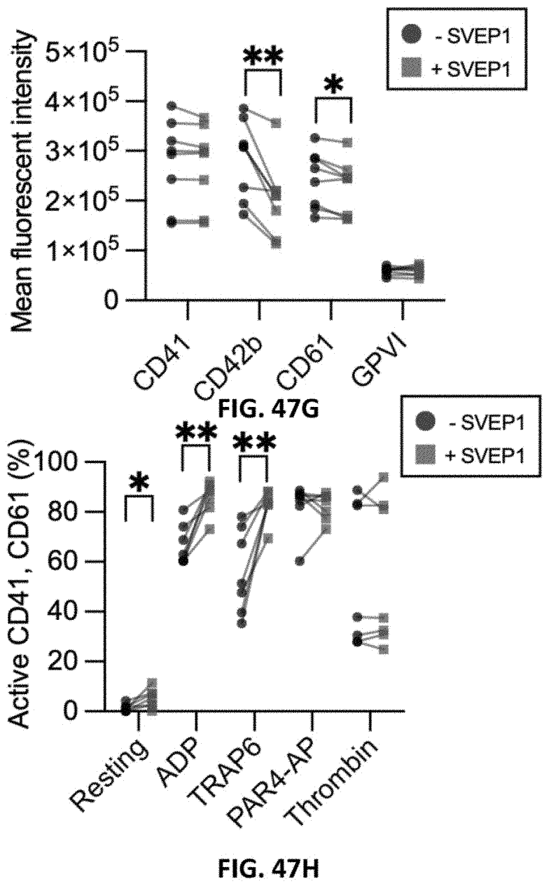

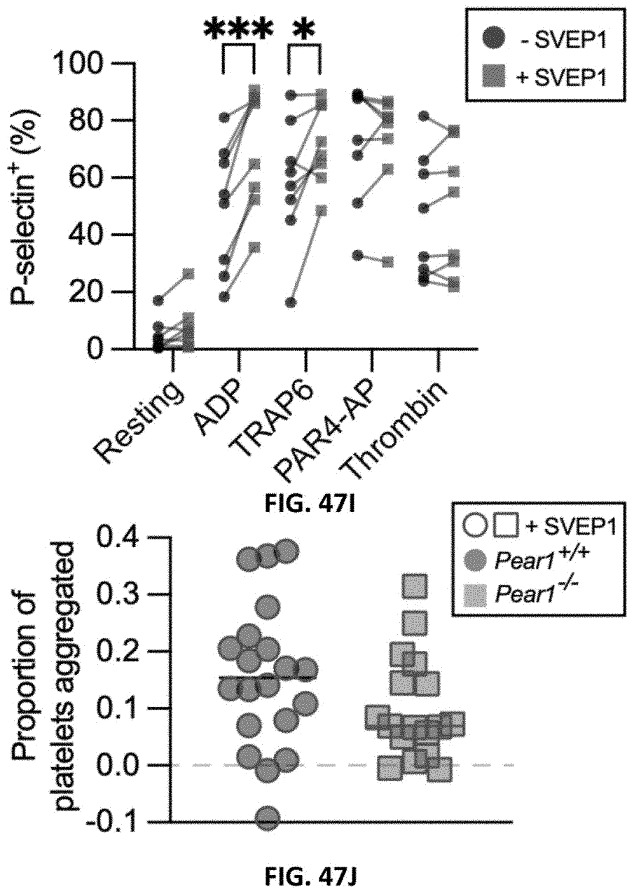

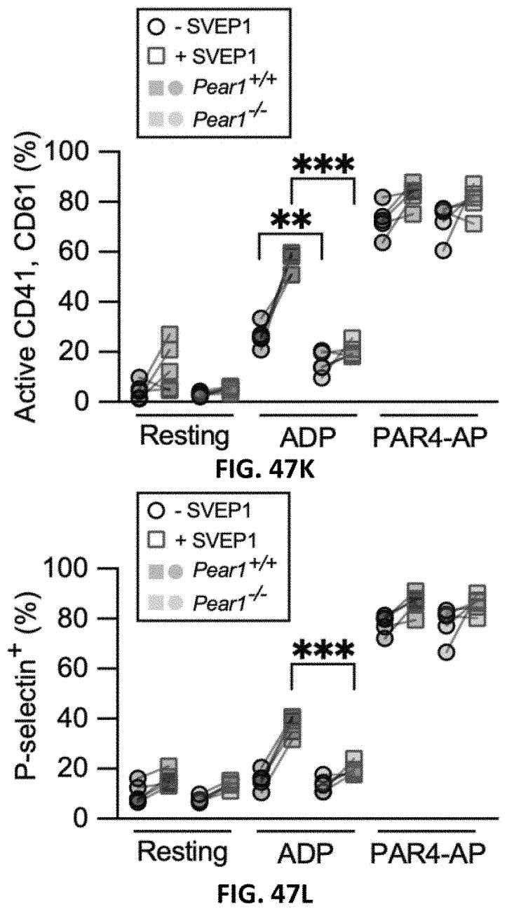

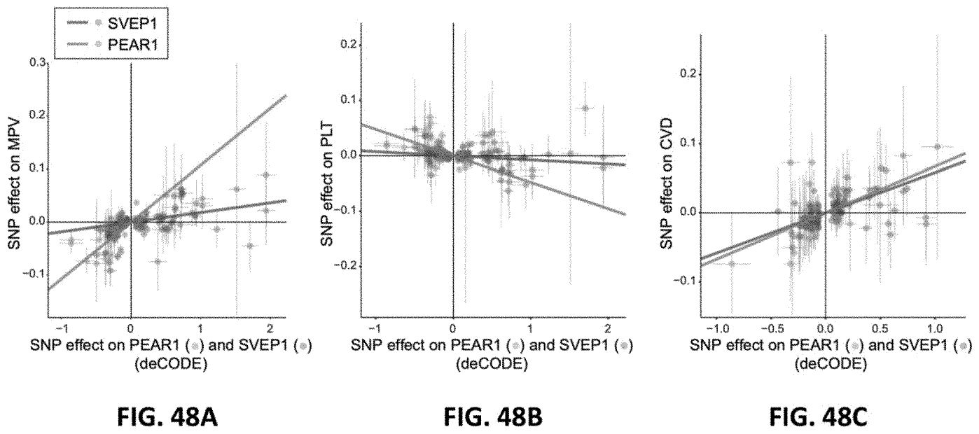

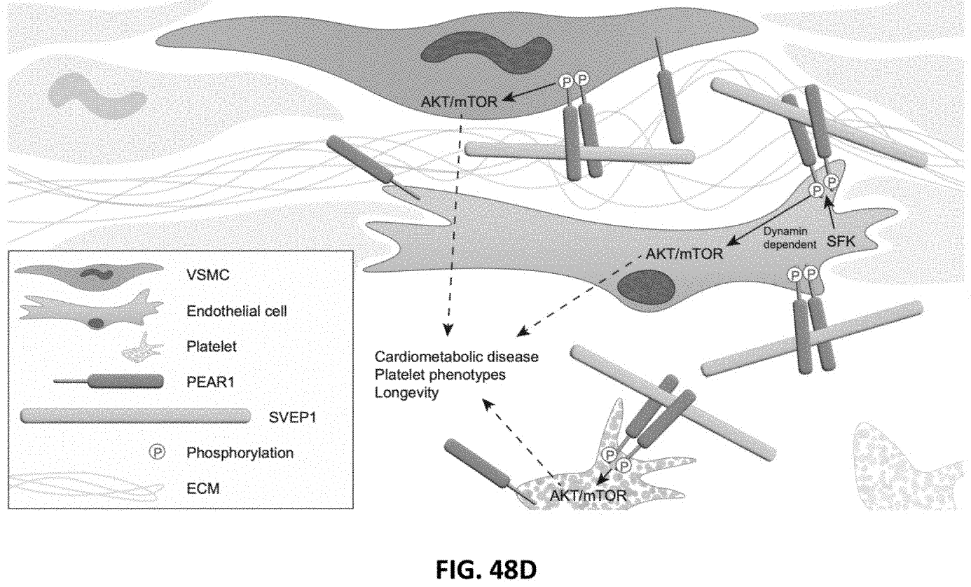

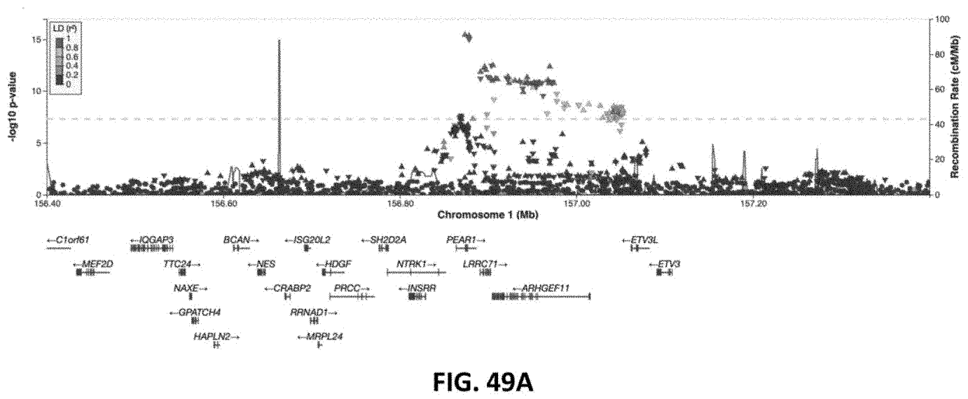

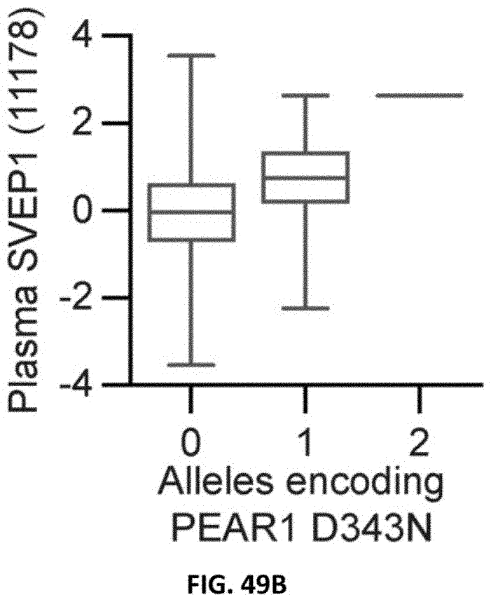

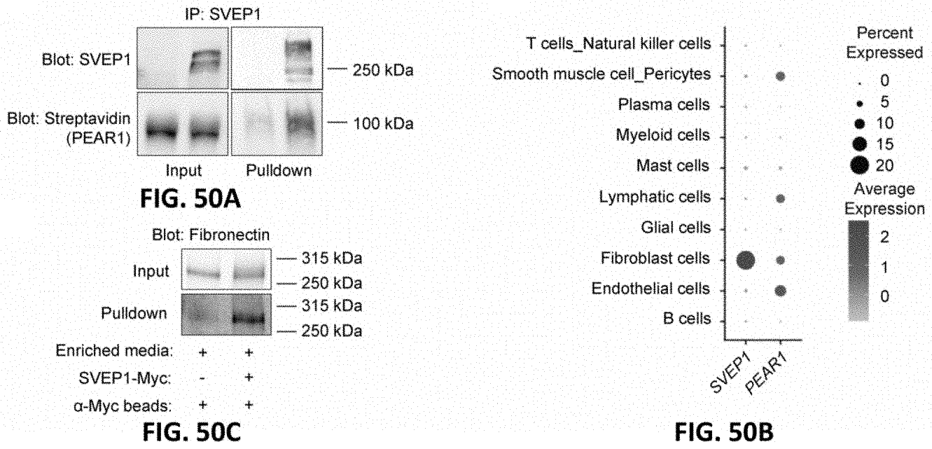

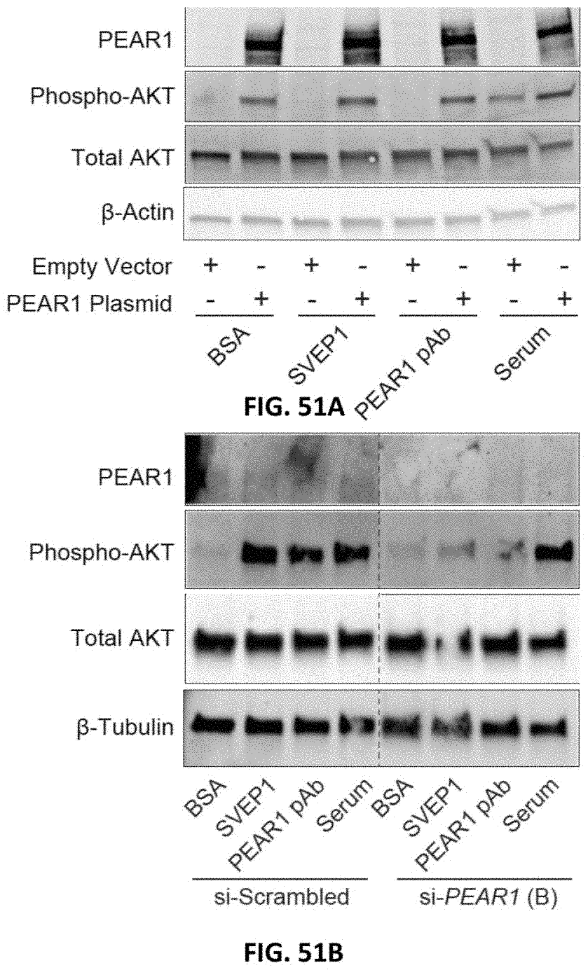

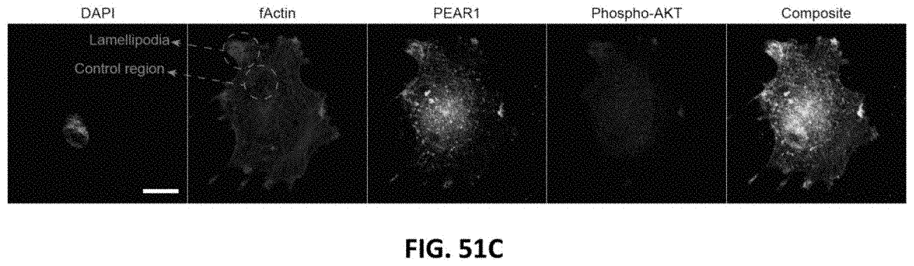

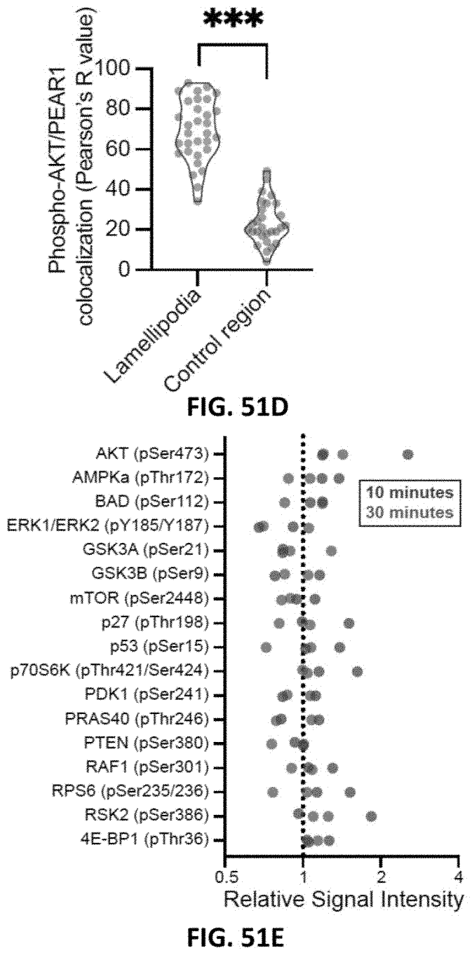

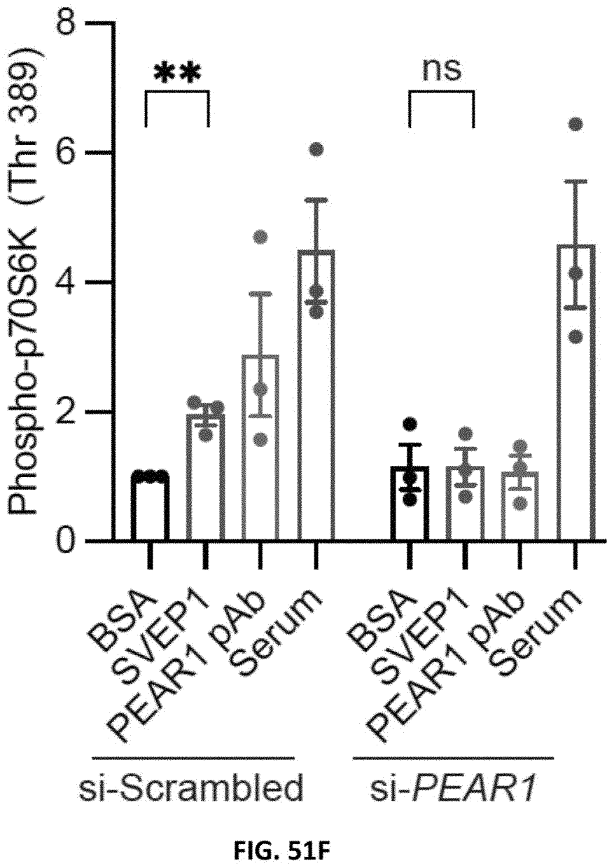

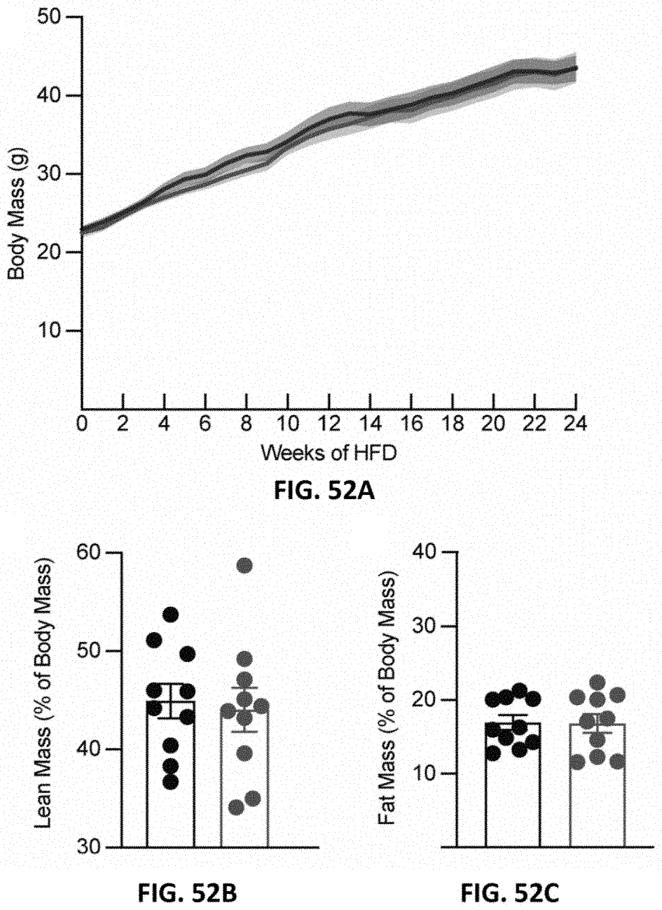

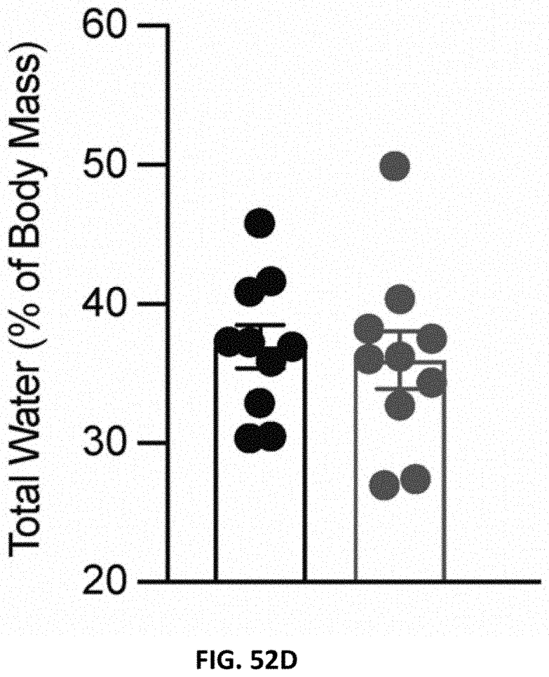

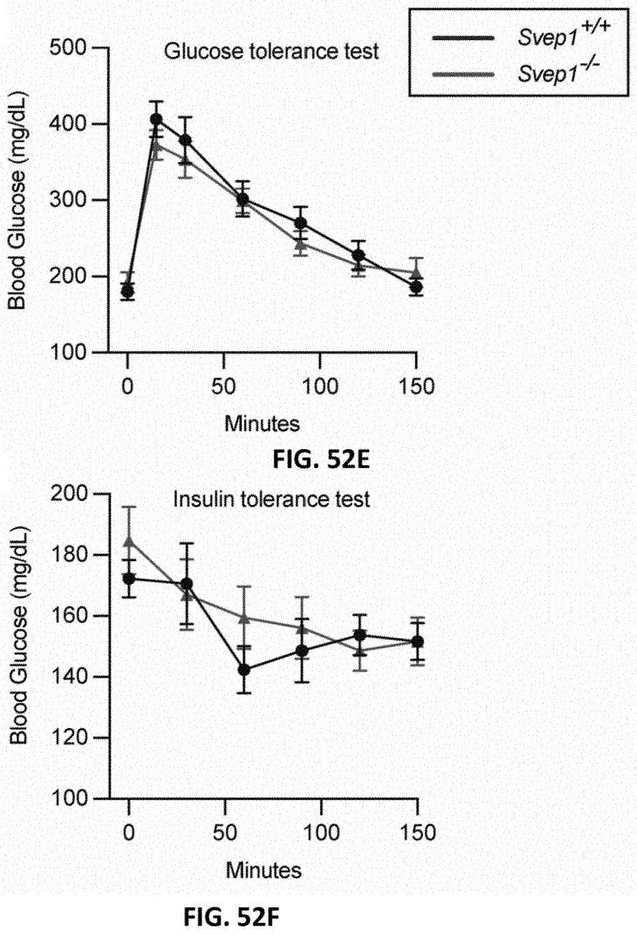

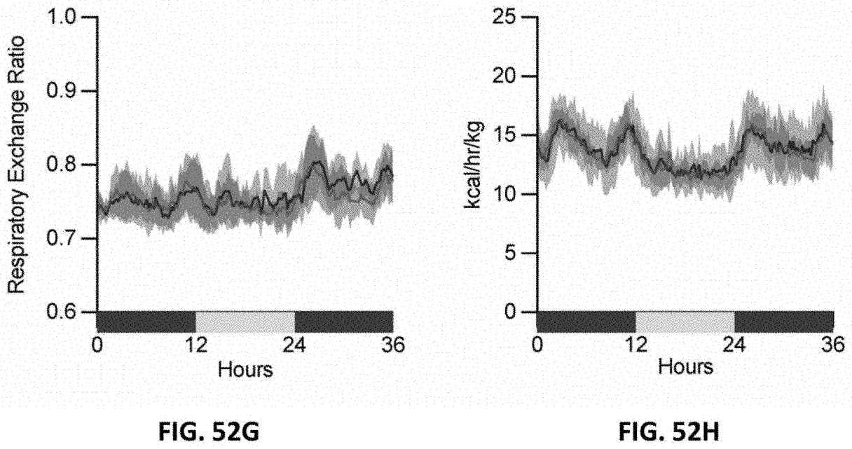

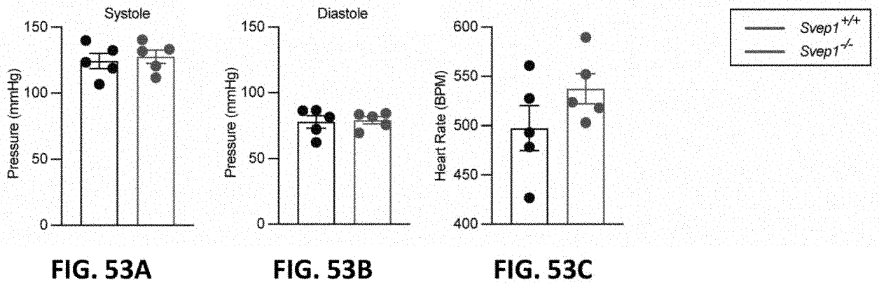

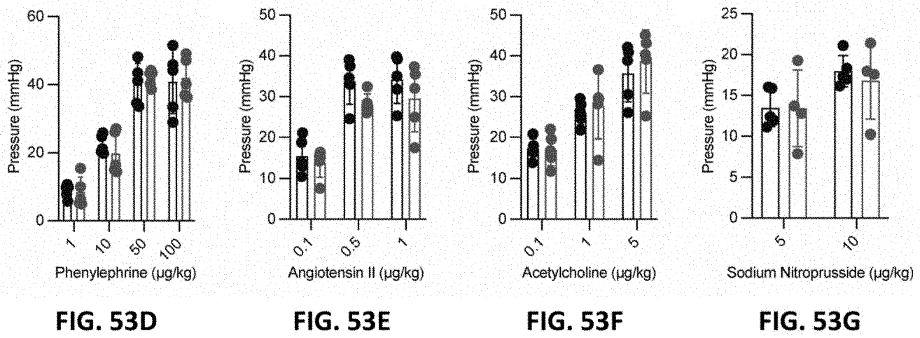

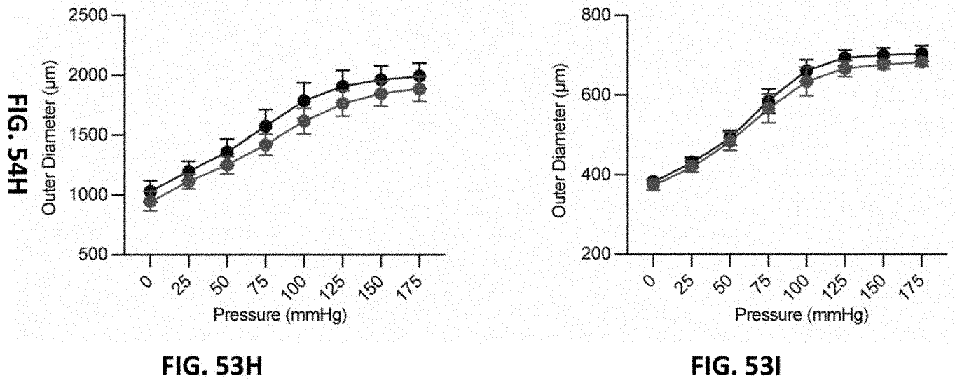

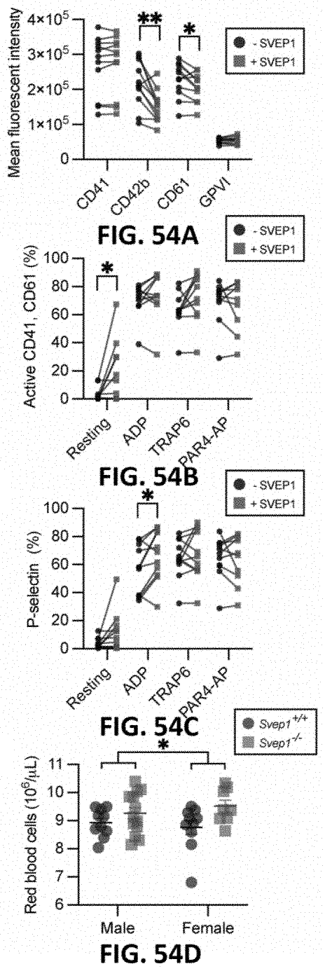

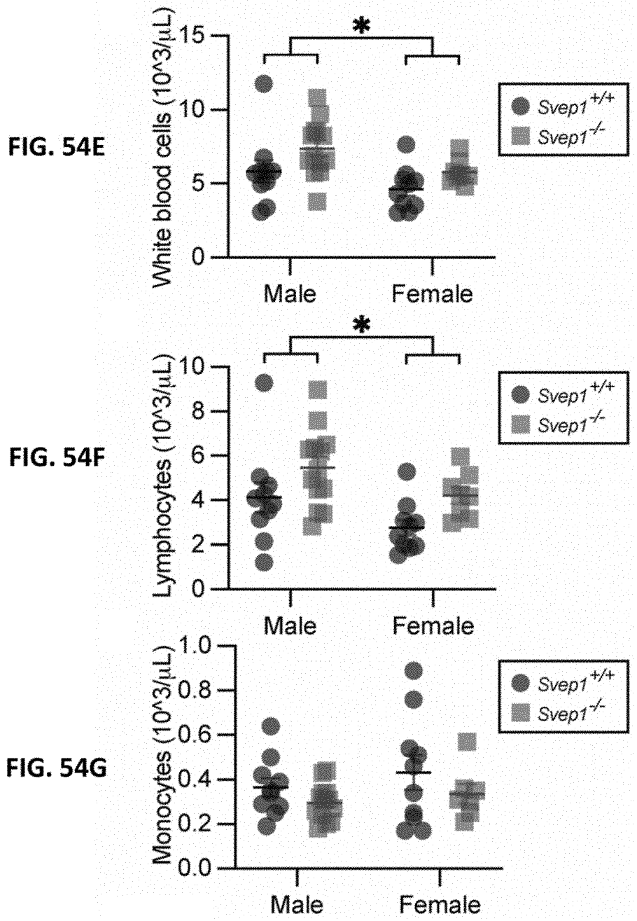

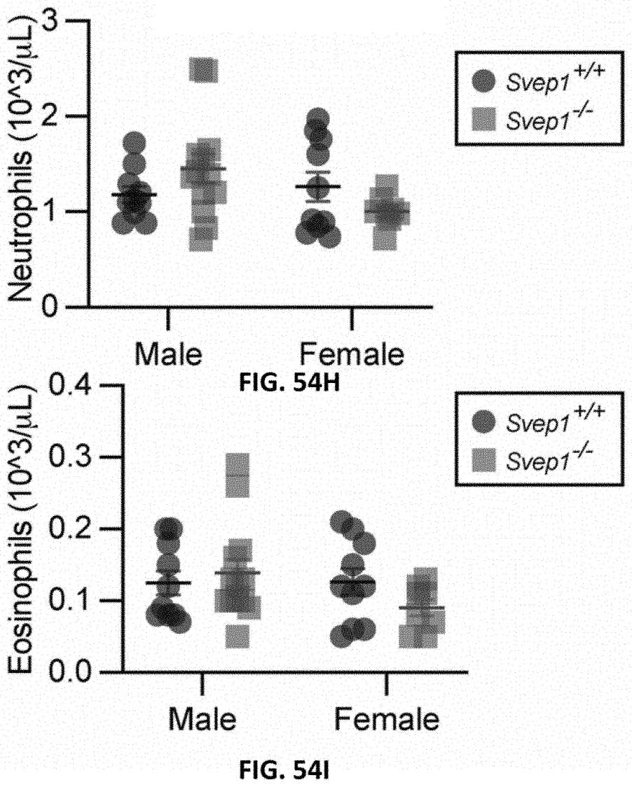

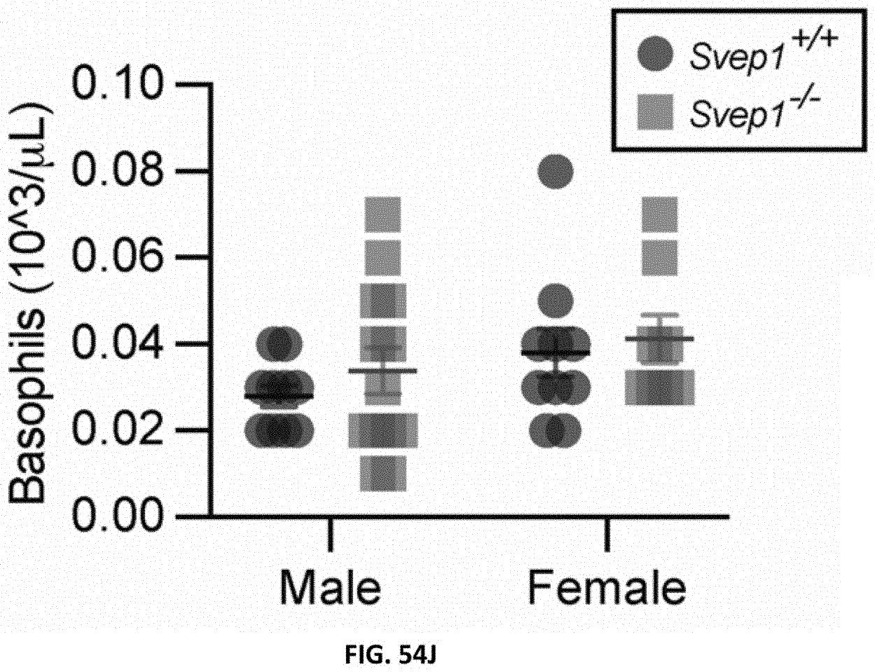

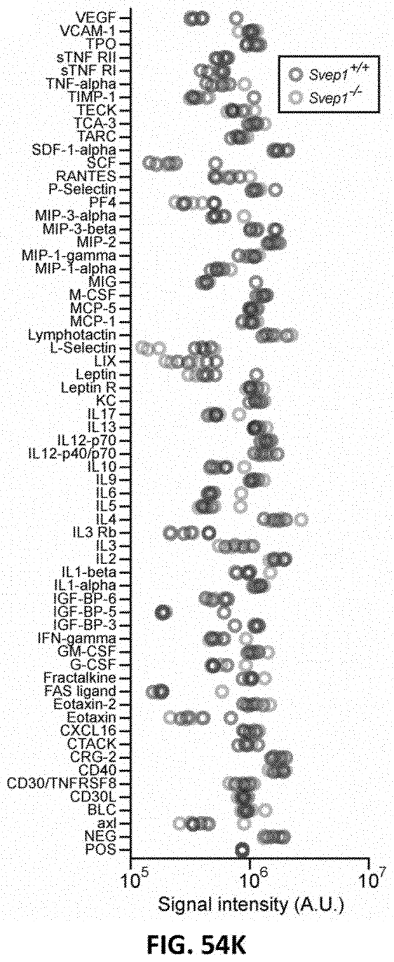

Pearl 1 is phosphorylated by SFK upon its activation. To test if SVEP1 can induce Pearl 1 activation and phosphorylation, platelets were exposed to immobilized bovine serum albumin (BSA, a nonspecific negative control protein), immobilized SVEP1, or soluble Pearl 1 polyclonal antibody (pAb, a positive control). Immunoblot assays revealed a robust phospho-tyrosine signal corresponding to 140 kDa, the expected mass of Pearl 1, after pulling down Pearl 1 from lysates of cells exposed to SVEP1 and Pearl 1 pAb but not BSA (FIG. 45A); this result is consistent with activation of Pearl 1 by SVEP1. Activation of downstream AKT signaling was then tested by probing the platelet lysates for phosphorylated AKT (pAKT). Consistent with Pearl 1 activation, both SVEP1 and Pearl 1 pAb induced AKT phosphorylation in platelets, but BSA did not (FIG. 45B). The response of Pearl 1-expressing HUVECs and hCASMCs (FIG. 44E) to SVEP1 was then tested using similar techniques. Serum-containing media was used in these signaling assays as a Pearl 1-independent, positive control for AKT signaling. AKT signaling was activated upon exposure to SVEP1, Pearl 1 pAb, and serum-containing media in both cell types (FIG. 45C, 46D), relative to BSA controls. Neither SVEP1 nor Pearl 1 pAb activated AKT in 293T cells, which lack Pearl 1 (FIG. 45E). Serum did activate AKT signaling in 293T cells, however, suggesting the AKT signaling axis was uncompromised in these cells. Reconstitution of Pearl 1 in 293T cells by transfection of a Pearl 1-expression plasmid resulted in constitutive AKT activation, such that SVEP1, Pearl 1 pAb, and serum had no additional effect on pAKT levels in these cells (FIG. 51A). Together, these findings are consistent with the hypothesis that SVEP1 signals through Pearl 1 to activate AKT. To directly test this hypothesis, transient Pearl 1 knockdown in hCASMCs using small-interfering ribonucleic acid (siRNA) was performed, since hCASMCs express Pearl 1 and are readily transfectable with siRNA. Cells treated with Pearl 1 siRNA had diminished Pearl 1 protein levels compared to negative controls (FIG. 45F, 51B) and were unable to activate AKT upon exposure to SVEP1 and Pearl 1 pAb. Serum-containing media was able to activate AKT signaling regardless of siRNA treatment, however, demonstrating an intact AKT signaling axis. These data demonstrate that SVEP1-induced AKT signaling is dependent on Pearl 1. Activation of AKT signaling by Pearl 1 is SFK-dependent. To test whether SVEP1-induced AKT signaling was also dependent on SFK, HUVECs were pretreated with the SFK inhibitor PP1 or carrier dimethylsulfoxide (DMSO) prior to BSA, SVEP1, Pearl 1 pAb, or serum exposure. As expected, PP1 abrogated the ability of SVEP1 and Pearl 1 pAb to activate AKT signaling in HUVECs (FIG. 45G). Pearl 1 signaling is also thought to depend on its internalization through a clathrin-dependent mechanism. To test whether SVEP1-induced AKT signaling was also dependent on this process, cells were treated with the dynamin inhibitor Dynasore or carrier DMSO prior to exposure to the stimuli. HUVECs pretreated with DMSO exhibited increased pAKT upon exposure to SVEP1, Pearl 1 pAb, and serum; however, the effects of SVEP1 and Pearl 1 pAb were abrogated by Dynasore pretreatment (FIG. 45H). These data demonstrate that the effects of SVEP1 on pAKT are dependent on SFK 209 and endocytosis, consistent with canonical Pearl 1 signaling. SVEP1's most recognized cellular function is to promote cell adhesion. To test whether Pearl 1-expressing HUVECs were able to adhere to SVEP1, the cells were seeded on tissue-culture plates coated with increasing concentrations of SVEP1 or plasma Fibronectin, a positive control, and blocked with BSA. After a brief incubation, the non-adhered cells were removed by centrifugation, and adhered cells were counted using automated microscopy. HUVECs adhered to SVEP1 in a dose-dependent manner up to 20 pg/mL SVEP1 (FIG. 451).

Pearl 1 and pAKT Colocalize to the Lamellipodia of Cells Grown on SVEP1

Previous studies have reported that Pearl 1 is localized to the filopodia and lamellipodia of cultured cells. These cellular structures are generated by actin polymerization and are sites of membrane protrusion and ECM adhesion. To test whether SVEP1 activates pAKT at regions of Pearl 1 localization, hCASMCs and HUVECs were seeded on SVEP1 and stained for filamentous actin (fActin), Pearl 1, and pAKT. Cells were imaged using fluorescent confocal microscopy and filopodia and lamellipodia were identified as bundles of fActin on the perimeter of the cells. hCASMCs treated with scrambled siRNA exhibited high colocalization of Pearl 1 and pAKT on lamellipodia and lower colocalization within the cell body, a negative control region (FIG. 46A and 46B). Similar colocalization was observed in HUVECs (FIG. 51C and D). Knockdown of Pearl 1 using siRNA diminished the colocalization in filopodia and lamellipodia (FIG. 46A and 46B). Together these data suggest that Pearl 1 on the surface of lamellipodia and filopodia activates pAKT locally when cells encounter immobilized SVEP1.

SVEP1 and Pearl 1 Activate Downstream mTOR Signaling

AKT is a central regulator of numerous signaling pathways; however, little is known about which pathways downstream of AKT are activated by Pearl 1. AKT-related pathways that may be influenced by SVEP1/Pearl 1 signaling were screened using an AKT pathway phospho-array. Given the temporal nature of kinase activation, HUVECs were exposed to BSA or SVEP1 for either 10 or 30 minutes before lysing the cells and assessing pathway activation. Elevated pAKT was observed in cells exposed to SVEP1 in each experiment (FIG. 51E), validating the methodology. Few changes were observed after 10 minutes of SVEP1 exposure; however, multiple phospho-proteins in the mammalian target of rapamycin (mTOR) signaling pathway were elevated after 30 minutes of SVEP1 exposure, including p70S6K, RPS6, and 4E-BP1 (FIG. 51E). Immunoblot assays of phosphorylated mTOR (Ser 2448) and the mTOR-regulated residue Thr 389 of p70S6K further support the activation of mTOR signaling by SVEP1 (FIG. 46C). Phosphorylation of p70S6K on residue 389 was also increased by the Pearl 1pAb and serum after 30 minutes of exposure (FIG. 46C). Transient knockdown of Pearl 1 by siRNA abrogated mTOR activation by SVEP1, as determined by immunoblot assay of p70S6K phospho-Thr 389 relative to total p70S6K in hCASMCs (FIG. 46D and 51F). Exposure of platelets to SVEP1 had similar effects on p70S6K Thr 389 (FIG. 46E), consistent with mTOR activation. Small molecule inhibitors of SFK (PP1), endocytosis (Dynasore), AKT (MK-2206), and mTOR (Rapamycin) were added to platelets prior to SVEP1 exposure to test whether SVEP1-induced AKT/mTOR signaling was dependent on the respective protein or cell process. Activation of AKT by SVEP1 was completely abrogated by inhibition of SFK and AKT and partially abrogated by endocytosis inhibition. Phosphorylation of p70S6K Thr 389 was dependent on SFK and mTOR and partially dependent on endocytosis and AKT (FIG. 46E). Taken together, these data suggest that activation of Pearl 1 by SVEP1 induces AKT and downstream mTOR signaling.

### Loss of Svep1in Mice is Cardiometabolically Well-Tolerated

AKT/mTOR signaling is activated by SVEP1/Pearl 1 and plays a critical role in numerous physiologic and pathologic processes, particularly processes related to metabolism and cardiovascular function. Heterozygous Svep1deficiency in mice appears to be well-tolerated into adulthood, but mice lacking Svep1during development fail to survive past birth and exhibit marked edema in utero. Given the biological role of AKT/mTOR signaling, the co-expression of SVEP1 and Pearl 1 in adipose and vascular tissue, the disease associations of SVEP1, and the interest in pharmacologically targeting SVEP1, the chronic impact of complete SVEP1 depletion on cardiometabolic phenotypes was characterized in post-developmental mice. Six-week-old Svep1flx/flxRosa26-CreERT2 (referred to as Svep1−/−) and control littermate Svep1+/+Rosa26-CreERT2 (referred to as Svep1+/+) mice were injected intraperitoneally with tamoxifen to delete SVEP1, as done previously. Mice were fed a Western high-fat, high-cholesterol diet (HFD) beginning at 8 weeks of age to induce cardiometabolic stress. Both Svep1+/+ and Svep1270 −/− mice gained body mass while being maintained on HFD. No appreciable differences were observed in body mass between the two genotypes of mice throughout the duration of HFD feeding (FIG. 52A). Similarly, compared to littermate controls, Svep1273 −/− mice had no appreciable differences in lean mass, fat mass, and total water, as determined by EchoMRITM. Svep1+/+ and Svep1−/− mice also had similar responses to glucose tolerance tests (GTT, FIG. 52E) and lacked insulin sensitivity, as determined by insulin tolerance tests (ITT, FIG. 52F). The metabolic activity of the mice was also tested using indirect calorimetry. Again, no significant differences were observed between Svep1+/+ and Svep1−/− mice in respiratory exchange ratio (FIG. 52G) or energy consumption (FIG. 52H). Collectively, these data suggest that whole-body deletion of Svep1in post-developmental mice with diet-induced diabetes does not result in an overt impact on body mass, body composition, glucose handling, respiratory exchange ratio, or metabolic rate and suggest that loss of SVEP1 is metabolically well-tolerated in adult mice.

Given the association of SVEP1 with hypertension, the cardiovascular manifestations of Svep1deletion were also tested using the same mouse cohort. Arterial catheterization was used to measure central blood pressure and heart rate in anesthetized mice. Significant differences between Svep1+/+ and Svep1−/− mice in systolic or diastolic blood pressure or heart rate were not observed (FIG. 53A-C). Vascular function was further explored by titrating the Svep1+/+ and Svep1−/− mice acutely with vaso-active compounds including phenylephrine, angiotensin II, acetylcholine, and sodium nitroprusside. Blood pressure was affected by the substances in a dose-dependent manner, and no significant differences were observed between Svep1+/+ and Svep1−/− mice with any drug at any dose (FIG. 53D-G). Similarly, no significant differences were observed between the two genotypes in vascular compliance of the ascending aorta or carotid artery, as determined using pressure-diameter measurements on dissected tissues (FIG. 53H and 531I). These vascular phenotyping data support the metabolic phenotyping data and suggest the whole-body loss of SVEP1 is cardiometabolically well tolerated in adult mice.

### SVEP1 Induces Platelet Activation

Given the human genetic associations of SVEP1 and Pearl 1 with platelet reactivity, platelet phenotypes of Svep1−/− mice and littermate control Svep1+/+ mice were characterized. Both genotypes of mice had similar platelet counts (FIG. 47A). The levels of the platelet surface receptor CD41 were modestly lower in platelets from Svep1−/− mice compared to Svep1+/+controls (FIG. 47B). This difference was variable between different mouse cohorts, however. To investigate platelet function, the response of platelets to agonists including ADP and protease-activated receptor-4 activating peptide (PAR4-AP) was tested. Platelet integrin a111433 activation (active CD41/61) was then measured, using an antibody that detects its active conformation, and platelet alpha-granule secretion, using an antibody that recognizes P-selectin (CD62). ADP or PAR4-AP-stimulated platelets from Svep1−/− mice had significantly lower integrin activation as compared to stimulated platelets isolated from littermate controls (FIG. 47C). Similarly, ADP stimulation resulted in significantly lower P-selectin expression in platelets from Svep1−/− mice, as compared to controls (FIG. 47D).

Human platelets adhered to immobilized SVEP1 under static conditions with and without the presence of 0.1 U/mL thrombin (FIG. 47E). Recombinant SVEP1 was added to freshly isolated human platelets or whole blood to test the effect of soluble SVEP1 on platelets. Soluble, recombinant SVEP1 induced spontaneous platelet aggregation and agglutination of whole blood, as determined by decreases in whole blood platelet counts (FIG. 47F). Platelets exposed to soluble SVEP1 had lower levels of receptor CD42b and CD61 (FIG. 47G), suggesting receptor shedding and platelet pre-activation. Platelets exposed to SVEP1 also had increased integrin a111433 activation under basal conditions and upon stimulation with ADP and Thrombin receptor-activating peptide-6 (TRAP6) (FIG. 47H). P-selectin expression was also increased in isolated platelets after the addition of exogenous SVEP1 and stimulation with ADP and TRAP6 (FIG. 47I). Similar effects were observed in platelets within whole blood upon exposure to SVEP1 (FIG. 54A-C). The SVEP1 variant (p.R229G) that associates with increased platelet reactivity in humans is also associated with increased plasma SVEP1, supporting these findings.

It was then tested whether the effects of SVEP1 on platelets were dependent on Pearl 1 using Pear1−/− mice. The effects of SVEP1 on platelet aggregation and agglutination were notably milder in platelets from mice (FIG. 47J) compared to humans (FIG. 47F), consistent with previous reports that murine Pearl 1 plays a less prominent role in platelet function than human Pearl 1. Upon activation with ADP, murine platelets derived from Pear1−/− mice and incubated with SVEP1 had reduced integrin activation (FIG. 47K) and P-selectin expression (FIG. 47L) compared to platelets from control mice.

It is therefore concluded, using three methodologically independent techniques (mouse models, exogenous SVEP1 assays, and human multi-omics) that SVEP1 promotes platelet activation, likely by signaling through Pearl 1. Many of the effects of SVEP1/Pearl 1 on platelet activation were potentiated by ADP; this finding is consistent with previous Pearl 1 studies and the GWAS associations of SVEP1 and Pearl 1 with platelet response to ADP stimulation in humans.

Given the platelet phenotypes in Svep1−/− mice, additional hematological phenotypes in these mice were assessed and it was found that Svep1−/− mice had higher red blood cell (RBC) counts (FIG. 54D) relative to control Svep1+/+ mice. This is consistent with the genetic association of SVEP1 with human RBC phenotypes. In addition, blood from Svep1−/− mice had greater total numbers of white blood cells and lymphocytes (FIG. 54E and 54F). The total numbers of monocytes, neutrophils, eosinophils, and basophils were not appreciably different between Svep1+/+ and Svep1−/− mice (FIG. 54G-J). Whether plasma cytokine levels may explain the hematological differences between Svep1+/+ and Svep1−/− mice was assessed using a cytokine array. No significant differences in plasma cytokines were observed between Svep1+/+ and Svep1−/− mice (FIG. 54K), perhaps reflecting the modest effects of Svep1on immune cell populations.

### SVEP1 and Pearl 1 are Causally Related to Human Platelet Phenotypes and CAD

It was then asked whether SVEP1 and Pearl 1 causally relate to human traits and disease. MR was used to test the impact of plasma SVEP1 and Pearl 1 on mean platelet volume (MPV) and platelet count (PLT). It was found that genetically determined increased plasma concentrations of both proteins associated with increased MPV (FIG. 48A; SVEP1 P=5.1×10⁻⁶0 ; Pearl 1 P=1.8×10⁻⁸) and decreased PLT (FIG. 48B; SVEP1 P=0.015; Pearl 1 P=2.3×10⁻⁵). Similarly, genetically encoded changes in plasma concentrations of both proteins are positively associated with the risk of cardiovascular disease (FIG. 48C; SVEP1 P=4.5×10⁻¹²; Pearl 1 P=0.0051). These data demonstrate that both SVEP1 and Pearl 1 causally relate to platelet traits and CAD. The effects of the proteins are concordant, consistent with the hypothesis that the two proteins interact to influence disease.

### Discussion

Recent genomic and proteomic studies have implicated SVEP1 and Pearl 1 in a variety of overlapping human traits and diseases. Understanding of the mechanisms of these proteins has been limited, however, since little was known about their molecular interactions. Previous studies have reported an interaction between Pearl 1 and high-affinity immunoglobulin epsilon receptor subunit alpha (FcεRIα); however, the differing expression patterns of these two proteins, the inability of FcεRIα to activate platelets, and the lack of conservation in mouse suggest FcεRIα is not a physiological ligand of Pearl 1. Similarly, the only protein known to interact with SVEP1 is integrin α9β1. Both SVEP1 and integrin α9β1 play a role in lymphangiogenesis, however, Svep1−/− mice die much earlier than Itga9 −/− mice (at birth vs postnatal day 14, respectively). Svep1plays a similar developmental role in zebrafish, but itga9−/−larvae do not phenocopy svepl−/− larvae and zebrafish Svep1lacks an integrin α9β1 binding domain. These findings suggest the SVEP1 may have additional interactions related to its role in development and disease.

Here, evidence is provided that SVEP1 is a physiological ligand of Pearl 1. In addition, their binding occurs with a stronger affinity than the other interactions reported for each protein. The observation that Pearl 1 D343N impacts plasma SVEP1 levels in humans led us to test the causal relationship between plasma Pearl 1 and plasma SVEP1, since an inverse correlation would suggest Pearl 1 can sequester plasma SVEP1. Indeed, genetically-encoded plasma Pearl 1 levels are strongly inversely correlated with plasma SVEP1 levels. SVEP1 and Pearl 1 physically interact and immobilized SVEP1 activates canonical Pearl 1 signaling in a Pearl 1-dependent fashion. It was also found that mTOR signaling is activated downstream of SVEP1/Pearl 1-induced AKT activation; these findings are summarized in FIG. 48D. It is unclear whether activation of AKT/mTOR by SVEP1/Pearl 1 is directly responsible for their causal disease and trait associations; however, these pathways are well known to contribute to platelet biology, cardiometabolic disease, and longevity.

Several independent studies have reported associations between SVEP1 and Pearl 1 in cardiovascular disease and platelet phenotypes, yet causality is more difficult to assess. Here evidence is provided that both proteins causally relate to human cardiovascular disease and platelet phenotypes using MR and mouse models. Mendelian Randomization can be used to test causal relationships in human biology and disease without the resource constraints and ethical limitations of clinical trials. This method relies on SNPs within a population that influence a quantitative exposure, such as plasma protein concentration, and an outcome of interest. A critical assumption of this technique is that the SNP exclusively influences the exposure. Most SNPs comprising the genetic instruments in this manuscript are non-coding; therefore, their associated differences in plasma protein concentration are likely a manifestation of the quantitative differences in protein production rather than functional differences. Proteins are known to leak from tissue to plasma and rigorous techniques have demonstrated that SVEP1 behaves in this manner. Taken together, this suggests plasma protein concentration may be a proxy for tissue levels of the protein. The mechanisms of SVEP1 and Pearl 1 ingress and stability in the plasma are unclear; however, the variables that regulate this process are randomly distributed across the cohort according to Mendel's law of independent assortment and therefore should not be a source of confounding. Given that plasma protein concentration may reflect tissue protein concentration, we conclude that the causal relationships of SVEP1 and Pearl 1 described in this study are not limited to explanations pertaining to the plasma. Nevertheless, the effects of the proteins on CAD, platelet volume, and platelet count are concordant, consistent with the disease mechanisms of SVEP1 and Pearl 1 being interrelated.

Several studies have independently concluded that increased SVEP1 is deleterious in humans. A single study in mice contrasts these conclusions by reporting that Svep1haploinsufficiency increased atherosclerosis; however, the results were difficult to interpret due to confounding introduced by differing proportions of males and females in their control and experimental groups. A previous study avoided this source of bias and directly contradicted their conclusions using the same model in addition to complementary mouse models and outcomes. SVEP1 is critical for proper development in mice, but our findings suggest that it may be dispensable in the adult animal, any biologically significant adverse cardiometabolic phenotypes in aged, metabolically challenged Svep1−/− mice were not observed. The human population variance of genetically encoded SVEP1 and Pearl 1 levels suggests a safe therapeutic window exists to target SVEP1 and/or Pearl 1 and potentially reduce their associated disease burden. The interaction between SVEP1 and Pearl 1 occurs within the extracellular space, making this interaction an intriguing target for pharmacological intervention. Additional studies will be necessary to further characterize the mechanisms by which SVEP1 and Pearl 1 influence disease and evaluate the potential of therapeutically disrupting their interaction.

What is claimed is:

1. A composition for treating or inhibiting a vascular disease or thrombosis within a patient in need, the composition comprising a Pearl 1 antagonist compound.

2. The composition of claim 1, wherein the Pearl 1 antagonist compound is selected from the group consisting of an antibody and a small molecule.

3. The composition of claim 2, wherein the Pearl 1 antagonist compound is configured to bind to or complex with Pearl 1 produced by endothelial cells, VSMCs, fibroblasts, platelets, and any combination thereof.

4. The composition of claim 3, wherein the Pearl 1 antagonist compound is further configured to block SVEP1/Pearl 1 signaling and associated downstream signaling.

5. The composition of claim 3, wherein the Pearl 1 antagonist compound is further configured to block a binding of SVEP1 to Pearl 1 and associated downstream signaling.

6. The composition of claim 4, wherein the associated downstream signaling comprises AKT/mTOR signaling.

7. A method for treating or inhibiting a vascular disease or thrombosis within a patient in need, the method comprising administering a therapeutically effective amount of a Pearl 1 antagonist compound, so as to reduce availability of Pearl 1 in the subject in need.

8. The method of claim 7, wherein the Pearl 1 antagonist compound is selected from the group consisting of an antibody and a small molecule.

9. The method of claim 8, wherein the Pearl 1 antagonist compound is configured to bind to or complex with Pearl 1 produced by endothelial cells, VSMCs, fibroblasts, platelets, and any combination thereof.

10. The method of claim 9, wherein the Pearl 1 antagonist compound is further configured to block SVEP1/Pearl 1 signaling and associated downstream signaling.

11. The method of claim 10, wherein the Pearl 1 antagonist compound is further configured to block a binding of SVEP1 to PEAR1 and associated downstream signaling.

12. The method of claim 11, wherein the associated downstream signaling comprises AKT/mTOR signaling.
